SEQUENCE LISTING (41 sequences)

SEQ ID NO: 1 - DNA, 20 bp, synthetic construct
  source 1..20: mol_type = other DNA
gtaacccgttgaaccccatt

SEQ ID NO: 2 - DNA, 20 bp, synthetic construct
  source 1..20: mol_type = other DNA
ccatccaatcggtagtagcg

SEQ ID NO: 3 - DNA, 21 bp, synthetic construct
  source 1..21: mol_type = other DNA
actgccacacctccagtcatt

SEQ ID NO: 4 - DNA, 21 bp, synthetic construct
  source 1..21: mol_type = other DNA
ctttgcctcactcaggattgg

SEQ ID NO: 5 - DNA, 22 bp, synthetic construct
  source 1..22: mol_type = other DNA
gatcgtccgagccaacatcaca

SEQ ID NO: 6 - DNA, 20 bp, synthetic construct
  source 1..20: mol_type = other DNA
ctaacagcccagccagcact

SEQ ID NO: 7 - DNA, 20 bp, synthetic construct
  source 1..20: mol_type = other DNA
gaatcattatgacggcagca

SEQ ID NO: 8 - DNA, 22 bp, synthetic construct
  source 1..22: mol_type = other DNA
tcatgtacaccgtgatgtggta

SEQ ID NO: 9 - DNA, 20 bp, synthetic construct
  source 1..20: mol_type = other DNA
cgtatcaccaaactcagaag

SEQ ID NO: 10 - DNA, 20 bp, synthetic construct
  source 1..20: mol_type = other DNA
gaagcaaagtttcctccaag

SEQ ID NO: 11 - DNA, 20 bp, synthetic construct
  source 1..20: mol_type = other DNA
ctggagaacctggtgcaaat

SEQ ID NO: 12 - DNA, 19 bp, synthetic construct
  source 1..19: mol_type = other DNA
cctcggaagccactaggac

SEQ ID NO: 13 - DNA, 19 bp, synthetic construct
  source 1..19: mol_type = other DNA
aatgtttcagtgcagagcc

SEQ ID NO: 14 - DNA, 18 bp, synthetic construct
  source 1..18: mol_type = other DNA
ttgggatgatgtcgggac

SEQ ID NO: 15 - DNA, 25 bp, synthetic construct
  source 1..25: mol_type = other DNA
ggaatacctgaacgtgcacatgaga

SEQ ID NO: 16 - DNA, 26 bp, synthetic construct
  source 1..26: mol_type = other DNA
gtgccatctgccatcacagagattct

SEQ ID NO: 17 - DNA, 25 bp, synthetic construct
  source 1..25: mol_type = other DNA
tgacaactttggtatycgtggaagg

SEQ ID NO: 18 - DNA, 24 bp, synthetic construct
  source 1..24: mol_type = other DNA
aggcagggatgatgttctggagag

SEQ ID NO: 19 - DNA, 20 bp, synthetic construct
  source 1..20: mol_type = other DNA
tctacgacacggtccaggag

SEQ ID NO: 20 - DNA, 22 bp, synthetic construct
  source 1..22: mol_type = other DNA
gaatactcccactcctccagtc

SEQ ID NO: 21 - DNA, 16 bp, synthetic construct
  source 1..16: mol_type = other DNA
gcggccgagaaatgca

SEQ ID NO: 22 - DNA, 19 bp, synthetic construct
  source 1..19: mol_type = other DNA
agtcgcagggcacacattc

SEQ ID NO: 23 - DNA, 20 bp, synthetic construct
  source 1..20: mol_type = other DNA
accaataccctgacctgctg

SEQ ID NO: 24 - DNA, 20 bp, synthetic construct
  source 1..20: mol_type = other DNA
ctatagctgctggggactgc

SEQ ID NO: 25 - DNA, 22 bp, synthetic construct
  source 1..22: mol_type = other DNA
aaggagatccaggtcttattgg

SEQ ID NO: 26 - DNA, 18 bp, synthetic construct
  source 1..18: mol_type = other DNA
accttcagccccgggtac

SEQ ID NO: 27 - RNA, 21 nt, Homo sapiens
  source 1..21: mol_type = other RNA
gcatcaagagcttcgggtatt

SEQ ID NO: 28 - RNA, 22 nt, Homo sapiens
  source 1..22: mol_type = other RNA
tagcccgaagctcttgatgctt

SEQ ID NO: 29 - RNA, 20 nt, Homo sapiens
  source 1..20: mol_type = other RNA
gctgcccactctacagtttt

SEQ ID NO: 30 - RNA, 21 nt, Homo sapiens
  source 1..21: mol_type = other RNA
aagctgtagagtggtcagctt

SEQ ID NO: 31 - protein, 1044 aa, Homo sapiens
MAPFATPMVQALTTTRIQRQAEGFQCWRECGTRRSPFEGKETCAHRYEARQRVDQILETRDMIGRCFVLSQDLAIRDELDGGEWKFCEGRPQGHEQFGFCQQGTAAAFSPDSHYLLFGAPGTYNWKGTARVELCAQGSADLAHLDDGPYEAGGEKEQDPRLIPVPANSYFGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEGVPWWVILLAVLAGLLVLALLVLLLWKMGFFKRAKHPEATVPQYHAVKIPREDRQQFKEEKTGTILRNNWGSPRREGPDAHPILAADGHPELGPDGHPGPGTA

SEQ ID NO: 32 - protein, 1137 aa, Homo sapiens
MAGARSRDPWGASGICYLFGSLLVELLFSRAVAFNLDVMGALRKEGEPGSLFGFSVALHRQLQPRPQSWLLVGAPQALALPGQQANRTGGLFACPLSLEETDCYRVDIDQGADMQKESKENQWLGVSVRSQGPGGKIVTCAHRYEARQRVDQILETRDMIGRCFVLSQDLAIRDELDGGEWKFCEGRPQGHEQFGFCQQGTAAAFSPDSHYLLFGAPGTYNWKGLLFVTNIDSSDPDQLVYKTLDPADRLPGPAGDLALNSYLGFSIDSGKGLVRAEELSFVAGAPRANHKGAVVILRKDSASRLVPEVMLSGERLTSGFGYSLAVADLNSDGWPDLIVGAPYFFERQEELGGAVYVYLNQGGHWAGISPLRLCGSPDSMFGISLAVLGDLNQDGFPDIAVGAPFDGDGKVFIYHGSSLGVVAKPSQVLEGEAVGIKSFGYSLSGSLDMDGNQYPDLLVGSLADTAVLFRARPILHVSHEVSIAPRSIDLEQPNCAGGHSVCVDLRVCFSYIAVPSSYSPTVALDYVLDADTDRRLRGQVPRVTFLSRNLEEPKHQASGTVWLKHQHDRVCGDAMFQLQENVKDKLRAIVVTLSYSLQTPRLRRQAPGQGLPPVAPILNAHQPSTQRAEIHFLKQGCGEDKICQSNLQLVRARFCTRVSDTEFQPLPMDVDGTTALFALSGQPVIGLELMVTNLPSDPAQPQADGDDAHEAQLLVMLPDSLHYSGVRALDPAEKPLCLSNENASHVECELGNPMKRGAQVTFYLILSTSGISIETTELEVELLLATISEQELHPVSARARVFIELPLSIAGMAIPQQLFFSGVVRGERAMQSERDVGSKVKYEVTVSNQGQSLRTLGSAFLNIMWPHEIANGKWLLYPMQVELEGGQGPGQKGLCSPRPNILHLDVDSRDRRRRELEPPEQQEPGERQEPSMSWWPVSSAEKKKNITLDCARGTANCVVFSCPLYSFDRAAVLHVWGRLWNSTFLEEYSAVKSLEVIVRANITVKSSIKNLMLRDASTVIPVMVYLDPMAVVAEGVPWWVILLAVLAGLLVLALLVLLLWKMGFFKRAKHPEATVPQYHAVKIPREDRQQFKEEKTGTILRNNWGSPRREGPDAHPILAADGHPELGPDGHPGPGTA

SEQ ID NO: 33 - protein, 244 aa, Homo sapiens
MDILVPLLQLLVLLLTLPLHLMALLGCWQPLCKSYFPYLMAVLTPKSNRKMESKKRELFSQIKGLTGASGKVALLELGCGTGANFQFYPPGCRVTCLDPNPHFEKFLTKSMAENRHLQYERFVVAPGEDMRQLADGSMDVVVCTLVLCSVQSPRKVLQEVRRVLRPGGVLFFWEHVAEPYGSWAFMWQQVFEPTWKHIGDGCCLTRETWKDLENAQFSEIQMERQPPPLKWLPVGPHIMGKAVK

SEQ ID NO: 34 - DNA, 34738 bp, Homo sapiens
  source 1..34738: mol_type = genomic DNA
acttggaagggtgaagggaggtgggcagatggcatcaagttgtaaatgccagggaagttaacgtggggaaggggaaatctggctgcttctctaaccacggcagctgttcctcccttgcccagtgctaggactcagagttgactcaagttgctctacaaagagctgggaggggcggggtaggataggggaattagtggaacatcccaccccttcaacagctatttgagggccagactggccagtgcctaaacttgggcggggtggggaaggaggaaactgatgttgataagaggtccataccccacattgaggtgatccaccaaagcttctgtcaggcaggtcgctgcagtgatagcctctcgcctcctcttttctggggagagggggaaaaggaattggaggggagacttggttttttgaggggtagtgttaaaggttccctacccatccattactattctgctagttgccgaaatttcttaactaatctctgaatgccggagctgaaaaggaccttgcaaaacatagcgcaacctctttcattttagagatgaggaaactggtaccctgggagggaaatggactcactcagggacgccaggaagtcagcggcagcgcccaggactcctgactcccaggcactctttctccactactcggccgcagaatcggtaactccgagatttccagcccggactcaggaaacaagtgccccacctccgttccggtggtttcaggcctccaagaagcccatctctggggggttccccacaccctcctccctccagagtcgctccttcccgccgccaggacgagaggctcccagggccaataggcagggcgctccggggcaggggcggggctaaggtgcggcaggtcggggattgggggcgttgccgaggggaccggagtattgcccaagggaccggagccagttggcaagggaaaggagtttcaaaaaaagcctctctgaaattccctctcccccatgcccgttgcccgctagctggcgtctttccctggatatggttagttactcgagatccaggctccgggctgaagctaggccgcggccgaagagaaaacggccgacaggatatttggctcaccctgcagctccttgcgttcattctgcttggcctggtgttcttttaggaggcgggacagcatggtcacgtcgggctgggggctgggtacgccgggccccctccggctccggaagctggaacgctcacctcggctccccggggccatgtcacgtgaccgctgtgtcacgtgaggttgggaaccgccctcttgagaccttgggtgaagatcttggcggggaagagaggtagagggttgcggagggcaggtgaagtgttctttattgggagcttagtttttggtgagtgttggaagaagtattttctaatacttatattgccaagccagtttttaacatttcctgtggatctcaggcttcgagggctggtggtgtcaggccgtctatcaccgtttaattgagggagcagaagctccctggcctcatttaaattattctccttccctctcctcacctcctggcagtgctagctggttctcagttgattcccagttccttccctcaaggagaggatgctttcctgtctctcccaaccctctttcttctcactaccccactgaagtctctcagctctgggtgatcattgtagagaagcattttaggcacttcagacagcctcctagggagcttggctaacttggcgggctgtgtgaatgtagaatttcatctctgtccttttgtggtcaggaatgcgaacccatttcagtgttagcagaagcgtaagtgctgtgccaaagtaaaaagtaatttatataaggcctctgtgaactaaaaagaaatgcttccgggattaactccagcgagaacaattataaacatcatgagcatattcttgctactggtacaatctctctcggtgcaccttgcactaacatgttgggtgactttgggcaagttgctcaccattctaggttatagttttctctttagtaaaactaggagaagggattggattaaatcggtggttctcaaccctggctaagcattagaatctaaacattagaatcacagaaatcacaaggcttataaaaatgccgtgattagccgggtgcggtggctcacgcccgtaatcccagcactttgggaggccgaggcgggcggatcacgaggtcaggagttcgagaccaggctgaccaacatggtgaaaccttgtatctactaaaaatacaaaatttagccaggcgtagtggcacaagcctgtaatcccagctactcaggagcctgaggcaggagaatcgcttgaacctgggaggcagaggttgcagtgagccgagatcacgccactgaactccagcctgggcgacagagtgggactctgtctcaaaaaaaaaaaaaaaaaagccttgattccagactaattaaatagaatctaaggagtgaagaccaaacatcactatatatatgtatgtatatatgtatgtatacgtatatatgtgtatatatgtatgtatacgtatatatgtgtatatatgtatgtatatgtatatatgtatatataaaattattattatttttgagacagagtctcgctctgtcgcctaggctggagtgcagtggcatgatctcagctgactgcaacctccacctcccaggttcaagcgattctcctgcctcagcctcctgagtagctgggattacaggcatgtgccaccacacctgactaatttttgtatttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttttttttgtatttttagtagagacggggtttcaccgttttagccgggatggtctcgatctcctgacctcgtgatccgcccgcctcggcctcccaaagtgctgggattacaggcgtgagccaccgcgcccggcctaatttttgtatttttagtagagatggcatttcaccgtgttggttagactggtcttgaactcctgacctcaggtaatctgcccgcctcggcctcccaaagtgctgggattacaggtgtgagccaccgtgcctggccttatcagtatattttaaaagctgttctggtgattctaatgtgtatccagggttgaaactcactgggctaaaccatatctaaaactatttccagctttggcattctctcattttgtgattcattggcatagttgaagttgacgtcattatatttacttgcagggggaggagaaatgggtgctgattgcattagaagaaaacattcttgaattttctgagtaagatttgagaattactgatcaatgattcattcaactatttgcagaatagcatcccaaatcctgggagatgtgcaactgtcaaaggattagtgaggagaagcacatgaaatgcttaagtggcagcataaagcaaaacattttaagtgccaaaataaaaattctaataaaaacgttagatactgtgtgtatgtgtctaaattatatgtgtactgtatcttctttctaaacatttcttagtactcatcatagtcaagcactttacatgtatgtattcatttaatccctagaacagttctgtgaaatattattacgagtatcccaaatttgcagatgaagaatctgaggtacaaggcagttaagtaatagagttcattagtggtagagctaggatacaaatccaggctatctgactccagagcctgagtcttaccgctgtatactgcataaaccacagttgagaccccaggagggtagacccagagcacttttctgggaatctctctgtctctaatcattatgcccctctagcttgctgggcatgagctgtagagaatcctggcaggggaggagtgtttgagaagaggctctgctccagagaagggctaatcaatagaagtgggaagagtgagagcatcacagagcagcctggctctgtcctccccactctctggctccagcttccaactgcagtgagcaagcaaaatagaggcagattggaaatgcctggtgcctcacctccagagctgtggggcctggaagagaaggacatgggttggaagtgtcaagataatggtgacatgggataagtgaaacagagggtgagggctaatgggggttagtgttgggggggtctcaaaagtgagtctaaatgttaagattaggccaactcagggagagtgtcaggcccatcctgtattaccctcacccttcttggcacaactgcctgacattttcacaatcggtgtagagactgccaggattctgcagacattagcctctcccttctctcccacacactggctgttcctttgccctagagctgagcagaggacatggggtatctgtatctagtgtccccatctgatctctctagggttatcctccatctgttctttttctgggcctttattgttcactgtctaaccttagtttctgctgcagttgaagctcagcttcacttgtccttggtagagatggacacaagtcgcatacatatctatctggagaatgactcttgaaatttgaagtctctttctgctcacctctccttacgatttcctactcagattattttcccttggactgaatgaatgcaactccacactcctttcttcactcatttccctcttcttttgaggtctctttcagagttagctttaattagcaagctccaaactgactggctgggccaaacagggctggaagctgctgagttgttaggggttggatgagggctgagggaggacacagagtgagtagacaaggcttcacatactgggagatctggctgggcttcaagagagggggttggtggtggtgtggctaagttccaaatgttggcctgaagccagtgctcaaagaaagggggcccttgagacagtccaaatggctccctttggtaagtgacctcccagtccttctcaagtgttaagggtttcttttggatcgtcaaagaccggagcggtcaagctgtttccccactgaattccctcaatcacatttatgttcttttcctcccagccactcccatggttcaagctttgactacaaccagaattcagaggcaggcagaaggattccagtgctggagaggtgagtgaagtaaaaaagttctcatggtgtgcatgttgggacggaaaagcctgaccttgggacataagctccaaggctctgttgccagatgaggtggagggagaagttagccctgaagtgtgtgttctggaagtgtttgcttgtaagctagagacaacagttgcaaaaagtgtgatttgagggagctgaaaaatactgatctcaaagtggggaagaagatgttgaaaagggaaggagctggagaaagcctcagcttccactcatacaaaagctaaagggctaaaatcttggctggatctggacatttctcaacgtctaaaattttggaaatttttataaagattattaatctttcatttttacatttaatttatttaaaaagttcagtttctcagtcatactagccacatttctttttctttttctttttttttgagacagagtctcactctgttgcccaggctgaagtacagtgtattaatctattccatggagtggagtggataatctattccatggattatcattttactttgttagtggtatccttagaagcacaaaatttttaaatttttttttttttttgaggcagagtctcactgtgtcccccaggctggagtgcagtggtgctatctctgctcactgcaggctccgccttctgggttcaagcgattcttgtgcctcagcctcctgagtagctgggattacaggtgtgtaccaccacgcccagctgatttttgtatttttagtagagatgggtttttgtcatattggccaggctggtctcaaactcctgacctcaagtgatccacctccctccctcccaaagtgctgggattaccatgtctggcttggaaattattttgaaataattatagatcagaggaagttgtaaaaatagcacatgaagtcttgtgtacctttcactcagtttcccctaatggtgacatcttatgtaactgtagcataaaatcaaaaccaaaaagttgatattggtacagtattgttaactggcctgcagacctcactcagttttcaccattttttacatgcatttatttgtttgtttgtagttctgtgcagttttatatcttgtatagatttggataatcaccaccacaatcaagatacaaaacccatcaccacaaaggaacccccttgtgctattcctttatgtttgtccccacccccctccatccttgtcccctggcagccagtaatctggtcttcatttctatagttttgtcattttgagaatggtatgcgagtggaataatacagtttcagcattttttgtttggagacagggtctcactctatcacccaggctggagtgcagtggcaagatcatggctcactgcagccttcacctcctgggctcaagtgacactcccgcctagcctcctgagtagctgggaccacagatttggctaacttttctattttttgtagagatgggggtctccctatgttgcccaggctggtctccaactcctgggctcaagtgatcctcctgccttggcctctcaaagtgctgggattgcaggcatgagccactgtgcccagctcagcattaatttttaatttaactaattcctaagctcttgactgaaatacaagaagttctctaacagtttatttattttaatattgagcttaccgcattctctggatccttctagtttcttttttttttctttttttctgatgtggagtctctgtcacccaggctggagtgcagtggtgccatctcagctcactgcaacctccgtctcctgggtttaagtgattcttgtgcctcagtctctggagtagctgggattacaggtacccgccaccacacccggctaatttttgtatttttagtagagacagggtttcaccgtgttggtccggctggtcttgaacttctgatctcaggtgatccgcctgcttcggcctcccaaagtgctgggattataggcgtgagccaccgcgcccggccccgtctagtttcttaatttccctcttcacctacgatattatcttccactccaacattctggtctcatttctccttgagagaaatctacatgtctaaatttactaggctggtctagcacgctcttgtgtgttcccctccctcctttgcccctctatttatagccaggctaattttgggtggcctctctctctcttctttcctgatctttcctcctgtggtggtgaggtgacttctcaaatatttggagagaggaggtcagaagcagattcttggcatctgatttcagccctggatcacagaagccagtggagtgggaatggagacaggcagaagctgcaggtgcagataggaggcagcttgggctctaaaggcattttgagctgggtcgggggcggggggacctgggcagggagtcagtagtcccagttctgtcctaattttgcaattctgcattcccatgtcagctcttctctactgtctggggctctgagatattaaaaaggatggggagggcatggtgaaagtagaatcctggtgccagccctgctgacagcatatgtatttccttatagtacctgtttagagatgtgttagtgctctggaggggatagccacaggtgtagtattggaaaacagagggccagacttccaaatgtctgttaacttatccaaggcaaagactgtcccagggcagcagagtaagaacccactttttttttgttttcaaagaagtataatcctgaacaatgaagtaggaaagacagaacacaggaagaggaaggaggtaggacacttattggaacttttaagaaagggaaagagaagaaagaatcgtaagaatatgatagtgtttgaagggcagagacaacactagaaacattgagaaatactctgagaaagattccaagtgtggcagagacaagaatgatgacaaaatagaatttgggatgagacaaaatcagatagtgagagagagaagggaagatggacagatgtatattcacaagaccaacaccagtaagcaaggggagtaggaaggggaagtgggagcattcgaggttcccattatgccaaattatttcctgtctctccttctggccccatttctgtatcggagttataaatagcagagagttggaaagtgtccccccacccccttgcctctgtcccagcctgagggaaagggagaggaagagggacaggccaatgggtccctgtggagatcccatctcagccccacccaggtcctgctgagccagtccaggactctgccccctcccatcccctttcatggataggaaatgtgcagtcctgggacgggtctggtagctggggacaccctttacatccctctgcctcttgggtccagtctctttcatctttgccttctttgacacccactcccctccccactgcttaatttcctcttcctgtaatcatccccagtcgttttcttttctcccttcattccatcccttgtcaattaatctcttgcccttctttcttcctctctattcctttcctttttccatttctccatttgctccccgtatctcccgagtttctctctctcttcttgcctctttttctctgttcccttgaatcctgacgatgtggctagcactgctgtggtcattgccgggctgggggcgggggatgggataggatgggggagggcagcggtctgatcccaacagcagaaagagtgctctatgtgaccatgggggaacagggagcactaagatgccacgctgcacccaggcccaggacggctcccctttcatttcctctctatctgcacatctctcttcccaggttgtcttttagcgtcttcccaacttctcatctcttaccctccttcctctgtttcagcccctctctttctatctgtacttctctccctccgcattccaaggcgccgcctccaccactcccggggtggggatggggttgggggagaaggggaggagagcgccgcgcaggggcggagccggagacggtgctgggcttggggggcgtggtggtggggggtcagcaaggctagtttccatcccagccaccagcctgggcatccccttggagacgggcttgggtctccacctgccgcgggagcgaggggcggggccggaggcggggcctgagtggcgtccccgggagaggaggcgggagccggagtgggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggtgagtcaccgcacccgcccagagtcgccatgcccgagccacagatcgtccccctccccactctgtgggcctcctcatttctctgttttctagccccaccaagacctagactgcccacagacatcccacatcccaacctggagccttgcctcatctggcttgcgtctgaagctgcacttcccggccctgagaccagtattttgctttagggatgagttggaaagcaaggttcttgtcttggcagcgaaccatctccttcttctgggcctttcccccaacttgcatccttgatccagccccagggcctctggctcccctgcttcttccaagggctgaattccccaagggagggagactgtctgtctctgcttagaatgggaggagatggaaaggacatagaagttgagggtgccatgagagggatgcatgcagggcagactccagaaataacttcctgctagagcattgccatggatggaatgagggcagcagggcactggaaggccaggagagagcttccacttctgtggcttaagaccacgggaagattgggagaggatctgcaggtctgccaacctgcagtaggtggcttggtgatagagagtggcagcaaactgaaccctcaaagtactagtagcagtagtagtagccgcagcagctgtagcagtgagagagatccaggaaggatgctggccaggctgctccccttcctcctccttagcaaatttccaactccaggaatctcagcagctgggaagggccaggaggagtaaggggtggaggacaattctaattttttctaatcagttcaggacccatgggagatggatatacttttgtgaggggcctgtgactggtcatgttgcctgtatccttggctcttgctacatgtctgattgtaaaaagggaggccagaggtgaagaaagcttctcacctgctcctgctagggggcttttctctcttcaaccagtgcctaagccacattaagtatccattactgggatcaatgctgtccactgggactgtcttctgcctctactgtcggtctgggggcagggggcagggacaagagctcatttctcctcacttgcttggggagtgggggcctagctctaatctttcttcttccattatccctatcatctggtagcagggtcggtggtccccaaaactttgggagagatagaaagcaacggacttcatctcctcttctgtttaccatctgcttcctcattcacctttgctccctccctcccttcctccctccttctccatctgtcagagttcgaggactggaggcctttttaggacatgctgaactctctaagctatttccaggcaaattctaggttatttttaatagcttggtctcttgtcatttccccctcctctctgaaggtggcccctggttccgtctcccagagccaagctggggcctttcccagagggcctgactgcctcaccctgcttttgctccagcagggggtgctctgctgctggggggcggggggtatgtgagaggccaggcacctgctcagtccctagcttttgagttgcaggtggcctgccttagcactcactgatgaaaaaaacttcttgcctgttttgatgtcttttagtctagctctgggatgagactttaaggtctaacctttgctgtgtggttccagcctcatttacttccctcaactgtaaaaaggatataaacatagtattactacatagggttgttgtgcagattaagagttcttaatatatataaaatgcttagaatagtgcatagcccgcagtgagtgctgtgaagtgttagtagtattgctattcttgtattgtgattcacagagcgccttacagagattctggatccaaaggcttggctagagggcctccctggctgagccagccttccaggccaagcatcctccccagagggccacccagattgagaggggccaaagaggggctggacttgggctggggccctggagtgtgtggagaatcgagaagtgcagtggtcgtgggctactcctttgtcttcacttagctgagctcccagggggtccctctgccccccagctgccaacacttttttttttttttttttgcttttctctctgcagtggctacactgtggctgtccagaagactggggtggttagggcgtatggcatgaagccaggaaggagtgtgtgtggctggaccagaggtggagggactagagaggatgctgctgggtgctcttgttccactaaggatcgattggtctcttctccaccaagagcggactgggcatatctatgcactcagcttctttcttccacatgggcccctcccctcctccctacttttggcctccagaggagatgtgaacatagaacaaggataacttatctgggtgcttagctatgcactgaccagctgtgacactgggtatctctatgagtccacaaaatgtgtgtgttcagtaaacacattctgacactccctatggggcaagcacaaagatgaaaagacagccccgacactcagagagatggccacttctatgtttggaggctgggggtactgctgacttgcctgaaggttgccatttatttatgcagggctgtatcaccccgtttccttttctgcccagggtaccctcatctccccactctctccttccctttctggggtggtctcagtgttctagagacaggtcagtcactgggtggagtgacaaagtgttggagttaggcccatgtggatttgaattccagcatcactgcttaatgtctttgagtgagttttctcatctgaaagacaagaaaagaatccttatctcataggattgttctgatgattaaatgacataatgcatgtgacttgcctatcctggtgcttggcacatatgtggacagtgatgaatgttagtttcttatatccctggtgtctagcctcggatctgacgtcatagtaggtgctgaataaatatgatttccttgtctcaccagcgcctgacacagggcttggcatacaatagactctcaataagtagttgaatgccaaatgtgtcttctcttctctactactccctataccccttctctgtcttgacactggctctgacaagggatggcagctgctaagagatgaggaggagttgtgggaaggaagaatggctctctgccctccccctccaccccatcagagctggcacagtgccccacagatgcctgtctgtaatactgcctaacatggttttgggccttgccccccaggaagggagatggaggagaagagtgtgggagagaggcgttgaggtttgtcccactgccacttctgagtctctccttctgcaaagagaggacccatggagccagctgggtgtcagtcatcttacctcacccccgccttcactctggcttgggggttcagccccaggggacccaggcagcctccattcccagcactgtgctcccctggggaagacggcttggctgtgatcatggaaaattgtcctgccaagaaagttgtagctgggaaagaggctgagggggaggcaggagagaagactgggtgggggtggaagggaaggagaaatcatggacatggggagaaggaaggatggggaaggggattcaggatgtcgggaagagaatggggtagcattggaggcagaaggagaaacttgtccctacctccatgccagccagagtgactgatggaatcctgggctggcacagcttctgggaggtggggtctttgctgggtccctgatgagggggcagtgggtccgtatctagcctcttgcctggcctctgaagctggtccctgagccacactctgatgccagtctggggccctgttacttttgctcccagcattcttggcatttctggctgggtttcaactggactgggttggggagcagggcagagcttggggatggggccaaggaggggatagggaaggcctactcaggaacaggtgctgggaacaggcagttctttcaaaccagcactgttggcctggctgcttgggttggcgtgtatgtgtgtgtgtgtgtgtgtgtgtgtatctactgtgtatgttgatcccttatccagatagtatgtacatgcaacgtgatgactgcatgaccaagcatattaatttgtccttgccagggtttgagaaaactgacatttgccccttctctttagtccttgaacactctctttagtactgaggggttgggcctgggcagctctaatgagattgggtcattctgacctctaactcctgtccctgtccctgcccctgccccatcttgcaggctgctggtgggtgctccccaggccctggctcttcctgggcagcaggcgaatcgcactggaggcctcttcgcttgcccgttgagcctggaggagactgactgctacagagtggacatcgaccagggaggtgtggccctgcatgaacagagtgggggaagcgtgtgagcggggaggagaggacttgggctcctcttccctcccctaattcccagtgtcctgcctctagctgatatgcaaaaggaaagcaaggagaaccagtggttgggagtcagtgttcggagccaggggcctgggggcaagattgttgtgagtattgcttctcatgactgaatgcacggatggggtgtgtgtgtgtgtgtgtttatggtgtgtgcatacgcataggtgtgcttagagaacacaagttaggaatatggtatgattccaagtacatcagggagatataaaaaggtgtgagacatggtccttgtccttataaatgtaaaaatgtctgtccattcattcatccatccatttgtcaaactcttactgagaaccttttaagcatcaggcattgtgctagttactacaggggaaggctcatgcctgtaatcccagcactttcggaggccgaggcaggtggatcacctgaggtcaggagttcgagaccagccggaccaacatggcgaaaccctgtctctcctaaaaatacaaaaaaattagccgggcgtggccgggtgcggtggctcacgcttgtaatcccagcactttgggaggccgaggtgggtggatcacgaggtaaggagatcgagaccatggtgaaaccccgtctctactaaaaacacaaaaaattagccgggcgtggtggcgggcgcctgtagtcccagctactcagagaggctgaggcaggagaatggcgtgaaccggagaggcggagcttgcagtgagctgagatcgcgccactgcgctccagcctgggtgacagagcgagactccgtctcaaaaaaaaaaaaaaattagctaggtgtggtggcaggcgcctgtaatcccaggtactcgggaggctgaggtaggagaatcacttgaacctgggtggaggaggttgcagtgagctgagatcgcaccattgtaccctagcctgggagacaagagcaaagttccgtctcaaaaccaaccaaacaaacaaacgaaaaaaccagagctctctgtttctctctctctctctatctttcagtaacacgcatagatacacaattaccaatacagatcactgtggggcagaatctggttcatgttaagtgagtggtctagtctccagtctataaaagtccaaaggaggagtagagagaagacttctgcagaggggatgatttgagccaggctttaataataggtaatacctagcctgtgcaacatagtgggacctcatctttataaaaaataaaaacaaattagccagtcatggtggtgcatgcctgtagtcccagctacacaggaggctgaggtgggaggatcacttgagccctggaggtcgaggctgcagtgagccatgattgtgccactgcactccagcctgggtgacagagtgagaccttgcttcaaaaaaaaaaaaaaagtaatacttggagagtgaagcggacaggaagttctttgcagatgagatggtgacacttacaaaggtccagggacagggccaagcttggcattttggaggactgtgacatgatcagggagacacacatcctatgtggtggcttaattgtgtcttttggctccaggcagaatgtggaacaaggagatctccatttgagggcaaggaagtgggtgcagacaggttgctgggttatgcatggacctgtgtaacactggcagggtaatggtgcttgagtggtgccggcataggggtgtgtgtgtatgtgtgcatgtgcatgtgcatgtgagcacacatgtatcagtatctgccaaatctctgcatatgggcagcatgcctcaagcaggtccctggcccacagagtgaaatgatccccatcccttcctcccccagacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaagggtgagtcactcctcgggaaggggagaaggggaccaaaacctcctcttacctcagagacagggttggggatggcacatggccaagcatgaccacatgtgcactgctgtatggccccagggcactgccatgccttccaccccattgagctagtgcacacatgaatggggggtgcctcctttccctcgcacggccaagtgttcctcaacatgctggcatgggccccaagtgcacgctgggcctgcagctggggcctgcatgctccaacacactagcccacacctcatcactgccattcccgtctccgcacgctgctgctggctgagctgacactcggtgagtgtgatgccacatctgggggaccccaggaagcctgggttggggacagggtggggagagggctagaaagaagaggcagggcttccccgtgtgcctgtctaactcagtgtccggcctgaggggtgttccttgcgccctgccctgggcactaacaggtctgtccttgcaggcacggccagggtggagctctgtgcacagggctcagcggacctggcacacctggacgacggtccctacgaggcggggggagagaaggagcaggacccccgcctcatcccggtccctgccaacagctactttggtagggacctctccccggcccagaactgctctaaccctctgctcctctctcttgtcctctctctccatgctcccatccttctgtctctgtttctgtctctcaccttgtctctctctgtctttctgtctctggctgtgatctctctggtctctttttctctctccacctcttcttcttccaccattttctggcctttctgtggctctgtctccctactctgtggcccctactctggatgtcccctccctggtgtctcaccccaccccccacagggttgctttttgtgaccaacattgatagctcagaccccgaccagctggtgtataaaactttggaccctgctgaccggctcccaggaccagccggagacttggccctcaatagctacttaggtttgtaagctcccacctcctggactctaggggcatggcccagcctcccctccttccccagggaactcgacctttggtgccttataatctcctcctcccccaacacacacccagggagacatacattgggcccaaattgcagagaagagctgggtccaatgatcaggcctaagaggaggaggcccccagggtggtggcctctggggctgtgagccaggggtctccatggaggaagattcaggtggaatgagagggccagggctgaggatattttgggaaggacagtcctgtcttctagggggactttccctgaggggatggatggtgggcacatattgaagaaagggctaatgttgttggtaagtccctctcgttgtctcatctgcattcctctgcagaggaggaggaaaccaggcctgggagatgtttgggtgaagcaggcgctctctcactcccccttgtctccccctcatccatgtgaagacttcccctccctgccaggatgagggagttgggggaaagaggtgcactgggtgggattcgggcctgagagggacctctagctcttctagctccctgggtgtgggcagggtgaggccactgtgctcagcctcctacctgggctcctggccttctcagccatcacctttctctctcttgcccagtccctgaggctgacctcactgcactttttgtgccaagcttgtctctgggcctggtgggtgtgggaggctgccaggccctgtggggaggaagagctatccagctgtggtgctgatgacttggggggacctatcttttggctcttaacctaggggagggggcagggtgcaggggagctgtgacttggctcttaacctgtagggagggggcaggggctgggggagctgtgacacaccccagcttctgagtcttggggtgaagacttaggggtaagtcacccttcccccaggcttctctattgactcggggaaaggtctggtgcgtgcagaagagctgagctttgtggctggagccccccgcgccaaccacaagggtgctgtggtcatcctgcgcaaggacagcgccagtcgcctggtgcccgaggttatgctgtctggggagcgcctgacctccggctttggctactcactggctgtggctgacctcaacagtgatgggtgagtgggtagagggccgtgccacctgagggaggctgggtctagtagccccagtctggctgaggccacttagcctcctgctggctcctctggccagggaggacccacactgaatgtttccctctctccatagctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccaggtgtgacggggaactggaaaggctcagggagggaggggccacaggagggatggggaagcccctcagaggtcagggtgtggtcttctgaggactcagggagagagggtccctgagcttatgtctgagctgtaccatttaccagctttctgaccttggcaagttcctaacctttttgcgttagtaatatctgcagggagtggccaagaggattaaagatgatgtatgtagagtgcctgggattttgtagcctctcaataaaatagaaaacatacctgagtgactggggggagttgaggcctggatcttgtctgcaaggcccccagccagcgtgactgccttttccctgtgccctgcagatattgcagtgggtgccccctttgatggtgatgggaaagtcttcatctaccatgggagcagcctgggggttgtcgccaaaccttcacaggtgaggggagtcgctgggatgagggaatgggtgtgggtggaatcagcagaggcatcaggggaggcagaggcctgcgggaggtgggattgagggaggctgacagctggttctctaggtgctggagggcgaggctgtgggcatcaagagcttcggctactccctgtcaggcagcttggatatggatgggaaccaataccctgacctgctggtgggctccctggctgacaccgcagtgctcttcaggtgagcccctctcaaccttttccctccctgaggccgtcagcccctccctgtgactctgaccccgacctcagtgccaaatctaatgctgaagagtgtttcccagcctcatgttctcatgtttcttgtgctcttgactccccaatcccagggccagacccatcctccatgtctcccatgaggtctctattgctccacgaagcatcgacctggagcagcccaactgtgctggcggccactcggtctggtgaggtgggatcgggtggcacctggaccctggcagcttcccctgccctccctttgatcctttatctccccagtttggggctggggctgtgacaggatgtgacagatggggtgggggtaggggccttggcccatcagcctcgtttggctcaggagccacctttgcccccgcagtgtggacctaagggtctgtttcagctacattgcagtccccagcagctatagccctactgtgggtgagtgcggtcccccctctgtggctcgcccttttgggtttcccagggaggggggtcacttcgaggtggtagaagagcacccttggaatggggtgagctggagcagctctgcagctcagcagctcctcctttcccaacatgccacagccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggtggacactgaccccttggcttctgagggtcattttcatggctccatctcttttccctgattcctcttagctgctttttcccgagcacactcgtgcctccttctaagacctagacacgtgggaaacctgtcttctgagctcacttcctcctcatctgctgccctctcctgtcatttctgcactccctggaggaggaggaggtgcaaggggcttcatgtcccctcttccagctgaatggaggaggtggcagcttttatcatcacatctggtcctcaaagccctaaaggtttcccctgtcccctaccccagtctttctccctcctccttgctcagttctccacctctctcattcatctccctaatattctttccaagttttagaattggtcctaggtctggctggggcactgagcagggctgaggcctggggattgttccagtgaggaatagaccttcctcttcctaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagaggtgagcatgggttctggtttagcccagggggaggagctgggagggcaaagatcatggtccctccccagtgacaccaattcacagctcacagagccctttcacgtatgctactccagtaattcctcatatctctaggtgcctaaatgaccatgttcctagtcaaggggacagagctcctctactccatgtagtcattcagggggctcaggttgatgggattctgtaatcttcaaatgcggccttcaagctcttgaagtcacattcatcccattcagctggaagggagaacatgaggccccttgcttgcatggtttttatggctggcccagaagtggtgcccttgacatcttttcccgttctaaatccggaactcagttacatgcatgtgagctggggcagtgtggcccagctcttgacttgctgctagtctctaccatggcatggtagtgctcccatcactaccatgatgagaggacccagaggagaggacccagttcaggatcacatggctaataagcagcagatctgagagttgaggacaggttgtttaatcctgtgatactctaacccaccatatgccatggtgacaggtttgccccctgccttgccccctaggaatgccccttatctcatgtctctccccagatccacttcctgaagcaaggctgtggtgaagacaagatctgccagagcaatctgcagctggtccgcgcccgcttctgtacccgggtcagcgacacggaattccaacctctgcccatgtgaggggggcagagagcagggtgggggtgggagggcaagagttcaaggattgagagaaagccctctcaggaggaccagtcagagggaagggctgagcctgcagaaaaggcagaaggtggaagaggacctgccgatggacttggagactgagatgagggtcagtgatggggccacgggggctcccatggcaaggaatagcaatccttcacagtacactaataatatgcagtctacatacatctcattggttagtcttcaaacctctgtgaggtaggaattatattagtctcattttatagatggagaactgaagcctagagaggttaagttacttggccaaggtaagtaatggtggagctgagatgagaacggaggtcccctgactcccagtcgtgtgtacagaggcctgaggcttggacgggtgtgatggggacctggggtgggaagagaggctcagggagtgaaggcaggtactgggggagcagactgggcgggggaatggtagggggaggtgttcagaacttagcaggagtggggtctgactctccagggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggtgaggacctgggggcaggatggggtggggtcttgaggggctccagtaacccagactgaccttgccttctctcccattccaggagaagccactctgcctgtccaatgagaatgcctcccatgttgagtgtgagctggggaaccccatgaagagaggtgcccaggttggcacatctgcccttatccctacgtgagtagcctctccataagcccgtagaccacccctgactctaatctctttccacccttggcctaggtcaccttctacctcatccttagcacctccgggatcagcattgagaccacggaactggaggtagagctgctgttggccacgtaagccaggcggggccggaagggtgaggtgggggtagtcatattgacctcatctgaccccttgggaggtgcctgtgcctgatgcccatacttgcccctgcccactcaccaggatcagtgagcaggagctgcatccagtctctgcacgagcccgtgtcttcattgagctgccactgtccattgcagggtgagcctggcccaagggggcacctccattggagggagggggcattgacatttccaaaccttggccagggccctgccttcattgagccaggccccagaccttctcacccctgttctgacctctccacgccagaatggccattccccagcaactcttcttctctggtgtggtgaggggcgagagagccatgcagtctgagcgggatgtgggcagcaaggtcaagtatgaggtcacggtaagtgtcagggatgaggcccctccatggtcaccctccctccttggcacagaggagaggctgagctgtgtccccagggctggggctcttctaccatgtggcagcatgcagtttgaggcctctgtcctgcatatggaccttggctgtgggaaggtgttcctgctggggcctcatttgaccatttcctggtcattctgtctggctgtctcacctgctggtgtggtaggccatgagagtccagggaagcttctctgctgtgggcctcagctgggggatgggaacatggggcggggggttaatgtctctccctagaactctgcctttgcttggctggggcctctcctcacaccttccagggacacttccagggtttcccggatttggtgggtggagagaatggtgcctggtggggtctaggatcctgagtatttgttgaaattgtgtttcctgagggccttctcttcagcccttgttccttccatctctgtggatttaggatccacttgaaagctgagttccttggctgggcacggtggctcatgcctgtaatcacagcactacaggaggccaaggtggcaggatcacttgagcccaggagttagagacaagcctgggcaacatagatcctgtctctacaaaaaattaaaaaaaaaaaaaaaagaaaaactgaggtctctcccctcatactctctttttcccacaggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggtgaggcttaggtgggggtgggggttagatgtggggacagatgttatggggagtaagggtagtgagtgcagtgatggggcaggaggaaggcggtggggaggagattcgatcttagcactgctgtgattggagggacactcactaagaccccccttccgtgtccaggatgtggacagtagggataggaggcggcgggagctggagccacctgagcagcaggagcctggtgagcggcaggagcccagcatgtcctggtggccagtgtcctctgctgagaagaagaaaaacatcaccctggtgagggcaggccagactcggtgcagccagagctccggggtgctgcgtgtccaggaagggtacctgttgagacacatgatggtctgggtgcaagttgaccagatgttcccatcacaattcataggagggggtgcccgggaggcagctactgacactgcagacccccttcctgctcagcccctgcaattgctcactctagaacaggtcactccacctgaagccagttagttagccaagcaattgattcacattacgtttatataaagcattacattttatacgtgatgggatggggttagcccatcctcacactttgaggtctgtttgtcgttcttttgaactcttataagataaaaaagtcaatttgtgtaaggacacttgcttgatttttacagtattaaaaatgttaagttcttttttgttgttgttgagacggagtcttgctctgtcaccaaggctagagtgcagtggcacgatctcagctcactgcaacctctgcctctggttcaagcgattctccttcctcagcctcctaagtagctgggactacaggtgcatgccaccacacccggctgatttttgtatttttagtagagacggggtttcaccatgttggccaggctggctggtctcgaactcctaacctcgagcgatctgtctgcctcggcctcccaaagtgctgggattataggcctgagccacgcgctcagcaaaaacactaaattctaaaagttctaataattaagacaaccgtatattatatgtgatttattaagtttaccccttttgagcagttttgctttccccttttctgcttgttttgcagagttgtgttgtttaggtgaaggtattgagcagatccgcacatgctctgtgcccctccctgctaggatggggctcgcagtgagaatgtggtggggacttagattaggaggtctttggcttgattgtaaggaggtggaggtaaaccccaggtagagccaaggagaccctttgcaggggaatgtcagcagctgctgcagtccctgctttgggccctcagctcccctgggagcatggcatgaggcaaactgaagaacgtcaggggatgtaggaacccatgtcgtggctgttgctctattacatggataatcctcccttctccacgtgaccgatttcctctgagaggcctcccttttaaatgggaactttgaaatagattctttttgttttcatgttaaaatgtatttatttatttaaatgacaaaaattatatttatcatgtataatacaatgttttgaaatatgtatatactgtggaatggctatattgagctaattaacatatgcatcacctcacatacttatttttgtggtaagaacacttaaaatctattctcttagtgattctcaggaatacaatatgttgttattaactacagtccccatgttgtgcattagatctcttaaatagatatctcttccttctgtctaactgaaactttgtatcttttgaccaccatctcctccatctccccagccacaccccgcctccactccagccctaggtaacaactattctactctctacttctatgagttcaactttcagattccacatataagtgagataatgcagtatttatctttctgaatctggcttactttgcttaacataatgtctaaagtagattcttttttattttattttatttttttattttttattttttaattttattattattatactttaagttttagggtacatgtgcacaatgtgcaggttagttacatatgtatacatgtgccatgctggtgtgctgcacccattaactcgtcatttagcattaggtatatctcctaaagctatccctccccactgcccccaccccacaacagtccccagagtgtgatgttccccttcctgtgtccatgtgttctcattgttcaattcccacctatgagtgagaatatgcggtgtttggttttttgttcttgtgatagtttagtgagaatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaaggtttgaactagtttacagtcccaccaacagtgtaaaagtgttcctatttctccacatcctctccagcacctgttgtttcctgactttttaatgattgccattctaactggtgtgagatgatatctcattgtggttttgatttgcatttctctgatggccagtgatggtgagcattttttcatgtgttttttggctgcataaatgtcttcttttgagaagtatctgttcatgtcctttgcccactttttgatggggttgtttgttttgttcttgtaaatttgtttgagttcattgtagattctggatattagccctttgtcagatgagtaggttgcgaaaattttctcccattttgtaggttgcctgttcactccgatgatagtttcttttgctgtgcagaagctctttagtttaattagatcccatttgtcaattttggcttttgttgccattgcttttggtgttttagacatgaagtccttgcccatgcctatgtcctgaatggtaattcctaggttttcttctagggtttttatggtttatgtctaacatttaagtctttaatctatcttgaattaatttttgtataaggtgttaggaagggatccagtttcagctttctacatatggctagccagttttcccagcaccatttattaaatagggaatcctttccccactggttgtttttctcaggtttgtcaaagatcagatagttgtagatatgtggcgttatttctgagggctctgttctgttccattgatctatatctctgttttggtactagtactaaagtagattcttttactaaaaaggcagaaggccttgcttttagacagcttggctaaggattcttgaatggctcaaaagtggcaaaataaaagataactcaaataattcagacactcttaaagccattcttctgtgtctggatcgtggtgtgatgaatataacataactttcaggcaaatgggtcgtgaggtggtgattgggccaaatggatttaggtcagtcctggaaagaacagagcctgagtcccctgtctacccaggagctcctgaggctacctcctcctccttcagcgagtttgggtttagggcaggggtgtggctttgccgaatatggacctgagttcaaatatttgctccaccactcattagctttgctagtgctgtgacctgaggcaggtcgcttggcctccttgggcctcctcagtttcttcctttgaaaaatgaggatgataatgtgcaccttgtaggactattgcgaagcttaagtgagataagtgcctgacaagtagtaggtgctcaataaatgggactgtcattaggtaacataagcatcacttggggaagctactctgcctgtgctgtcaagtaagagattcccttccatgctgccccctccacgcatttcaaagctgaatagctcagatttggggcaggcagctattttggatataagattagtttgagaagacttcctggaggaggggagtttgaggtcaagtaaggaaaatgggagagatggcagtctcagcagggttgagctgagtggcttagacccctgtctgtccttatccctaggactgcgcccggggcacggccaactgtgtggtgttcagctgcccactctacagctttgaccgcgcggctgtgctgcatgtctggggccgtctctggaacagcacctttctggaggtgaggataccactcggctctagtctgtgtttcttcttcctccaaagcccctccaggactcatttctcctcctccttcctcactgtccaaatgcagcatccccccccttttttttttttttgagacggagtcttcctttgtcccccaggctggagtgcagtggtgcgatctcagctcactgcaacctctgcctcccaggttcaagaggttctcctgcctcagcctcctgagtagctgagattacaggcacgtgccaccacacccgatatttttgtatttttcatagagacggggttttgccatgttggccaggctgatctcaaactcctgacctcaggtgatccaccctccttggcctcccaaagtgctgggattacaggtgtgagccactgtgcccagcccaaacgcagcatactcttaacccaaaccaggcagaaacatcctctgtgttgggcaccattacatttataatttcaggaggtaaggggaagggagggagacatggggcatttaggagaagggtctccttccctgttcccttaggagtactcagctgtgaagtccctggaagtgattgtccgggccaacatcacagtgaagtcctccataaagaacttgatgctccgagatgcctccacagtggtgagctgcagggttgggggataggtggggaggactctcttctttcctgctccctccagttcttgttccattgacctcttgctccattgaccccttgctccccagatcccagtgatggtatacttggaccccatggctgtggtggcagaaggagtgccctggtgggtcatcctcctggctgtactggctgggctgctggtgctagcactgctggtgctgctcctgtggaaggtgaggcttggaggtggggctgatgggggtggacttcctggttgggtgagcagcattttgtattgtggttctgtcacccacccacacactcatgtattcagcaaatctgcactgaacacttgccctgcatgtctcaggaactctattagtccctggatgtttcaaagataaatcagatgcaggctgggtgcagtggctcatgcctggaatctcagcactttgggaggcctaggcaggtggatcacttgaggtcaggagttcgagaccagcctggacaacatggtgaaaccccatctctacttaaaatacaaaattagctgggcgtggtggcacgcacctgtagatccagctacttgggaggctgaggcaggagaatcgattgaactcgaaaggtggggttgcagtgagccgagatggcgccattgcactccagcctgggtgacagagcgagactccatctcaaaaaaaaaaaaaaaaaaaaaagccgggcatggtggctcatgcctgtaatcccagcactttgggaggctgaggtgagtggatcacctgaggtcaggagttcaagaccaagctggccaacatggtgaaatcctgtctctgttaaaaatacaaacaaataaaaaaaattagctgggcatggtggggggtgcctgtaatcccagctacttgggaggctgagacaggagaatcgcttgaacccgggaggtggaggttgcagtgagccaagattgcgccactgcactgcaacctgggggactgagcaggactctgtctcaaaaaaaaaaaaaaaaaaaaaaaaaagaaatcagatgcagatctcgttctcagggattttatagtccagtgggatgggagtggtgagcctgtagtcaaggacaaggcaggaagtgttgagttgagaaaatggagtgggagtttagagggaaaaaagcttgagaggaggtagcactggaaccagtcctcagaggaaggtggggtagtagggtgaggatgtaaggggatgaggtaggtggggtccagcaggaccatggtaggcaaagtcgtggaggcagagaagtgtgatctgacttaactagaatgtggcagtgaaaactggaaaggtaaatgtggccaagaaggccaacaagcacggagaacggcctccaggggactgccctcatggcggaactcagagcttccacataccaggctctgcatggaagcattcttgtacacccagagctgtataccgaatcatactggtatccagggctcaatgtaaaagtatgcacatgcacacgtcagtacacggaatcacatacatcaccggggtcatatgctgagccatctgccacatcgggggctatgttcagaaccacacatgtccttggggctctggagaactgcccctgcctgtgcctgctctttgggaccaatcagagactgagccaccctttggcaagtgtgaatcatcagtagaccaggctctggagtggcatccaagctgttgggaaccttcccagtcttaggggagattgatgcctctgtgaaactggtcccaggatgcttgctccctgctcccatggaaggaagacagagcagagataggggatcctgtccttcctcaatctttccatcctctgcttcctctgccctgcttctccctcgacatcctagtgtggcttcttccatcggagcagccagagctcatcttttcccaccaactatcaccgggcctgtctggctgtgcagccttcagccatggaagttgggggtccagggactgtggggtaactgttgtgtgtgtgcatgtgtctagtgtgtgtgggtatgtatgtgtgtgaatataaggagaattcccagcattcaggggatcaggaatgttcagggtgaaaaatgagattgcatgagagtgatgtgaggtgagaggtaattagaagggtgcagatataagggtaagtctacacagcctgaggcgtgcacaaagggttggtgggctcatggggtgtgagcatgtgggagtgtggggaggtcgcatgcatgcttttttctatgggcatgagtgtgcaaggagatgaatgtgtaaggagatgccatgcagtctgagcatgtaggagtcctgggcctctgagtgtgagtgtgcaggggcacattcccaccctggtgtgtaagggccagcatggtcctcatttagggtggggctagactactgtattgatggacaagagggtattgctgtaaggggcgtgccgcccaaggtacaagtacacggggtgcatgtttatgaggcttgagaggtatcattccagagatgcaggggactaggaagccattttcttcctcaggtcttgtttgtgttctgggggacactattgggtctcttaagaagctagagggggtacaaatcttgagtccggtcctgatgctccaccctcaaggccctctttcaggggccaagggagatacccttggtcctgcctcttggacgcccttctggtctctctcttgctgtgaatgagaacctggggacacagcactgatgggagcaccgtgggtgctgcccaccacaccagatgctattcagcctatttcctttctgccgcagcagggatggtgagggtgaggagctgtgccgcgctaggcatctgctccagggaccgcagctcttcaggctcctcatggtctggcctggtgtccttatagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggctgctccttctctagtttcccctctcatctgaccttagtttgctgccatcagtctagtggtttcgtggtttcgtctatttattaaaaaatatttgagaacaaaacctctgcctctttgagtcttgctctggcatccccagcatctctgattctccctggtgcccccagctcaggaagaaggtggtagtggggagagagggtcaggggggcttggcagggatgcaggcaccatgacttttgtgaccagttcctagagacgcatgggtgtagcctcaggaggaaagcgagaggagctttaccatgggaacgaaggaaagggacaacattgggaggcaaacgttgggagactagtccagaaacttgcagttgaggatacaacagggtcctagtgtttcctccctgggtcagcgtagaagtagagctttttcactgtcgaaggcgggattgaagttagagattgccctggcagaggttggggagagagggactagtaggtggtacatgaatgtcttctctcattctgctaggtccctctcagtggaagatagataggctggtgggtggcttgttctaattggaggctggggaaggagcaaatgagtgccttggagcttgggaaagattatttgacagcctttcccatgatgtggggcccaacaggtagccacttcaagggagggggctgtcgttccatttggatttcggagaactgggcgttctcaagatccttccaggtctctctggtgaggcagcagccatccccaatgtgtttccaggtgggctcgaaaacttgctgccacatgaaggcccagcttccatatggttctgccacatgctcccagaaaaagagcacacctccctgggagggatgagagagttagtgtcacttggctccattctgaccctgctccctatcgcgctgatcaagacagtcaaacttcttggtgaaatgggagctgctggtctgagagttcatggagcaaactgtcgtgcccctgaccttttctgcatcccctgctaccgccttcaaagtgcatgtgaagaacctcataactgaaaggagtggctggtgggagatctttaaagcctgaccagaataaccgagggcatgcagaaggggaaactgagaaacaggagtggagggctcattgtattattattattattattattttttgagacagagtctcagtctgttgctcaggctggagtgcaatggcgtgatctcggcgcactgcaacctccacctctcaggttcaagcgattctcctgcctcagcctcccaagtagctgggattacaggcatgagccaccacgcccggctactttttgtatttttcagtagagacagggtttcaccatgttggccagtctggtttcaaactcctggcctcaagagatccacctgccttgcctcccaaagtgctggattacaggcgtgagccacctcgcccagctgggcttattgtattctgagatacacgcacaacctctctcacaaaacccagatgcatgcacaagctgcttcctggacatcgctgtgaacatcccagggaaacctcaggcccaactcttgactgaagctggggttcctcaagctcctcgcctcactatactctccctaggtgatctcaaacctagtaagtccaaaacagctttcttccatctcagcaaatggcacttttcatcagctctcccctcagttgctcatgctagaaacctggaaactattctgaacgttctctccacagtctgatggattctactctacttcccctactatctttacctagaacactcttttattttccccaccccgtccacactctgcttaatccctcagatctctctctctctctctctttttatttatttatttattttttttgatacagggtctcactctgtggcccaggctggagtgcagtggtgcgatcatagttcactgtaactttgaactcgggctcaagtgatcctcccacctcagcttcctgagtagctaggactataggcatgtgccaccacgcctggctagcttttgtatttttttttttttttttttttgtagagacggcatctcactatgttgccttggctggtcttg

SEQ ID NO: 35 - DNA, 20 bp, Homo sapiens
  source 1..20: mol_type = other DNA
accaataccctgacctgctg

SEQ ID NO: 36 - DNA, 20 bp, Homo sapiens
  source 1..20: mol_type = other DNA
ctatagctgctggggactgc

SEQ ID NO: 37 - DNA, 20 bp, Homo sapiens
  source 1..20: mol_type = other DNA
accaataccctgacctgctg

SEQ ID NO: 38 - DNA, 20 bp, Homo sapiens
  source 1..20: mol_type = other DNA
ctatagctgctggggactgc

SEQ ID NO: 39 - protein, 1816 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNHVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSSGRVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 40 - protein, 339 aa, Homo sapiens
MAAEDELQLPRLPELFETGRQLLDEVEVATEPAGSRIVQEKVFKGLDLLEKAAEMLSQLDLFSRNEDLEEIASTDLKYLLVPAFQGALTMKQVNPSKRLDHLQRAREHFINYLTQCHCYHVAEFELPKTMNNSAENHTANSSMAYPSLVAMASQRQAKIQRYKQKKELEHRLSAMKSAVESGQADDERVREYYLLHLQRWIDISLEEIESIDQEIKILRERDSSREASTSNSSRQERPPVKPFILTRNMAQAKVFGAGYPSLPTMTVSDWYEQHRKYGALPDQGIAKAAPEEFRKAAQQQEEQEEKEEEDDEQTLHRAREWDDWKDTHPRGYGNRQNMG

SEQ ID NO: 41 - DNA, 1682 bp, Homo sapiens
  source 1..1682: mol_type = other DNA
gcacttcgcgctcaagcgaccggatcttcaaaccgtgggagtggtgcggcggctagagtccctggactcctcaacctagggagctactcgcgagatgcctacgacttgtaacgggctgcctggtaaaatgagtctatggaaacggttgccagggccggctaacagcggctcccggaagtcctttgatgctttgttaacagtgaagctactggaccaatgaggtgcttcttccggttttgtccgcgctcgcctaattcttctttatcaaggttgcctttgaccccggaaaagagatcttccgggttcctctctccccaagatggctgctgaggacgagttacagctgccgcggctccccgagctgttcgaaactggtagacagttactggacgaagtagaagtggcgactgaacccgccggttcccggatagtccaggagaaggtgttcaagggcttggacctccttgagaaggctgccgaaatgttatcgcagctcgacttgttcagccgaaatgaagatttggaagagattgcttccaccgacctgaagtaccttttggtgccagcgtttcaaggagccctcaccatgaaacaagtcaaccccagcaagcgtctagatcatttgcagcgggctcgagaacactttataaactacttaactcagtgccattgctatcatgtggcagagtttgagctgcccaaaaccatgaacaactctgctgaaaatcacactgccaattcctccatggcttatcctagtctcgttgctatggcatctcaaagacaggctaaaatacagagatacaagcagaagaaggagttggagcataggttgtctgcaatgaaatctgctgtggaaagtggtcaagcagatgatgagcgtgttcgtgaatattatcttcttcaccttcagaggtggattgatatcagcttagaagagattgagagcattgaccaggaaataaagatcctgagagaaagagactcttcaagagaggcatcaacttctaactcatctcgccaggagaggcctccagtgaaacccttcattctcactcggaacatggctcaagccaaagtatttggagctggttatccaagtctgccaactatgacggtgagtgactggtatgagcaacatcggaaatatggagcattaccggatcagggaatagccaaggcagcaccagaggaattcagaaaagcagctcagcaacaggaagaacaagaagaaaaggaggaagaggatgatgaacaaacactccacagagcccgggagtgggatgactggaaggacacccatcctaggggctatgggaaccgacagaacatgggctgatcttcccacaacaccacaggactgcagggtgcacaactcccctgccaaggaaaaccatgcagtcctcccctccctggtctcctgcttcagctctgtacaacgagggcaaagatgctaaatcttgctttgcattcagtaaagtgtcaagtgattaagtgtgtatttgtaccctagatgatatgaaccagcagtcttgttttggcatcatcctcatcatgttgtattccagcttcttaagtggaaggaaaagagtgctgagaaatggctctgtataatctatggctatccgaattctctgaaaaaataataaaagtcccctctattatatgagcctgtacagaaa

(19) United States
(12) Patent Application Publication
(10) Pub. No.: US 2026/0248734 A1
(43) Pub. Date: Aug. 27, 2026

(54) COMPOSITIONS AND METHODS FOR TREATING METABOLIC DISEASES/DISORDERS

(71) Applicants: Achraf Noureddine, Albuquerque, NM (US); Charles Jeffrey Brinker, Albuquerque, NM (US); Eric Brey, Albuquerque, NM (US); Maria A. Gonzalez Porras, Albuquerque, NM (US)

(72) Inventors: Achraf Noureddine, Albuquerque, NM (US); Charles Jeffrey Brinker, Albuquerque, NM (US); Eric Brey, Albuquerque, NM (US); Maria A. Gonzalez Porras, Albuquerque, NM (US)

(21) Appl. No.: 19/160,247

(22) PCT Filed: Feb. 27, 2024
(86) PCT No.: PCT/US2024/017524

§ 371 Date: Aug. 27, 2025

Related U.S. Application Data
(60) Provisional application No. 63/448,492, filed on Feb. 27, 2023.

(51) Int. Cl. A61K 9/50; A61K 31/352; A61K 31/36; A61K 31/426; A61K 31/4439; A61K 47/68; A61K 47/69

(52) U.S. Cl. CPC A61K 9/5068; A61K 31/352; A61K 31/36; A61K 31/426; A61K 31/4439; A61K 47/6849; A61K 47/6913

(57) ABSTRACT

Provided herein is a population of protocells comprising a lipid bi- or multi-layer, mesoporous silica nanoparticles (MSNPs), a cargo, and optionally a targeting ligand and methods of using the same.

### PRIORITY

This application claims the benefit of priority to U.S. Provisional Patent Application Ser. No. 63/448,492, filed Feb. 27, 2023, the complete disclosures of which is incorporated herein by reference in its entirety.

### STATEMENT OF GOVERNMENT FUNDING

This invention was made with government support under grant R01 CA226537 awarded by the National Cancer Institute and grant F32 0DK122754 awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Feb. 25, 2024, is named “1863281WO1.xml” and is 77.927 bytes in size.

### BACKGROUND

Obesity, a chronic imbalance in energy homeostasis between energy intake and energy expenditure, is a public health crisis and continues to be among the most important medical challenges in various regions notably in the Tropical Pacific Islands, North America, Middle East and Oceania, but it also expands to numerous countries in Europe and South America. Obesity creates a greater than $190 billion burden annually on American healthcare with the percent of US medical expenditures devoted to treating obesity increasing 29% from 2001 to 2015. According to the most recent CDC statistics, over 70% of the United States population is overweight or obese, and more than 120 million people have diabetes or pre-diabetes. The development of therapeutic approaches that lessen or eliminate obesity-associated morbidity and mortality would have a transformative impact on the American population and healthcare spending.

There are two types of adipose tissue in mammals: white adipose tissue (WAT) which is specialized for energy storage, and brown-like adipose tissue (BAT), specialized for energy expenditure and heat generation. The significant capacity of BAT for energy expenditure may be a mechanism for the treatment of metabolic disease, but the small volume of BAT in adults suggests that the therapeutic potential is limited.

BAT have been detected in human WAT. These “brown-in-white” or beige cells can be induced in WAT. Engineering BAT or beige adipose tissue depots for subsequent transplantation has been explored as a treatment for metabolic disease in pre-clinical models. While investment has been made in designing therapeutics that target beige or brown adipose tissue, it remains difficult to maintain adipose tissue transformation in vivo.

### SUMMARY

The disclosure provides a population of protocells comprising a lipid bi- or multi-layer and mesoporous silica nanoparticles (MSNPs), a cargo, and a targeting ligand, e.g., a ITA7 targeting ligand. The disclosure also provides a population of protocells comprising a lipid bi- or multi-layer, mesoporous silica nanoparticles (MSNPs) and a cargo, wherein the cargo comprises at least one of forskolin ((13R)-1α,6β,9α-Trihydroxy-11-oxo-8α,13-epoxylabd-14-en-7β-yl acetate); 3-AR agonist CL-316,243 (5-[(2R)-2-[[(2R)-2-(3-Chlorophenyl)-2-hydroxyethyl]amino]propyl]-1,3-benzodioxole-2,2-dicarboxylic acid); rosiglitazone (Rosi; (RS)-5-[4-(2-[methyl(pyridin-2-yl)amino]ethoxy)benzyl]thiazolidine-2,4-dione; Avandia) or GW0742 ({4-[({2-[3-Fluoro-4-(Trifluoromethyl)phenyl]-4-Methyl-1,3-Thiazol-5-Yl}methyl)sulfanyl]-2-Methylphenoxy}acetic Acid); or peptides, proteins, antibodies, nucleic acids, or drugs that inhibit ITA7. In one embodiment, the MSNPs have a diameter ranging from about 1 nm to about 300 nm. In one embodiment, the MSNPs have a polydispersity index of <0.1. In one embodiment, the protocells have a ratio of lipid to MSNP of about >1:1. In one embodiment, the protocells are in an aqueous composition having an ionic strength of >20 mM but less than about 500 mM, e.g., less than about 50, 100 or 200 mM. In one embodiment, the targeting ligand is an antibody that binds ITA7. In one embodiment, the targeting ligand is an antibody fragment or a scFv. In one embodiment, the targeting ligand is covalently linked to the lipid layer. In one embodiment, the targeting ligand has a non-covalent linkage with the lipid layer. In one embodiment, the lipid bi- or multi-layer is biotinylated. In one embodiment, the lipid bi- or multi-layer is PEGylated. In one embodiment, the lipid bi- or multilayer comprises: (a) at least one zwitterionic lipid selected from the group consisting of 1, 2-dioleoyl-sn-glycero-3-phosphocholine (DOPC), 1, 2-dioleoyl-sn-glycero-3-phosphoethanolamine (DOPE) and 1,2-di stearoyl-sn-glycero-3-phosphocholine (DSPC); and (b) optionally, one or more additional electrically charged or neutral lipids selected from the group consisting of 1,2-dipalmitoyl-sn-glycero-3-phosphocholine (DPPC), dioleylglycero triethyleneglycyl iminodiacetic acid (DOIDA), distearylgtycerotdethyleneglycyl iminodiacetic acid (DSIDA), 1,2-dioleoyl-sn-glycero-3-[phosphorserine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-Oleoyl-2-[12-[(7-nitro-2-1,3-benzoxadiazol-4-yl)amino]lauroyl]-sn-Glyce-ro-3-Phosphocholine (18:1-12:0 NBD PC), 1-palmitoyl-2-(12-[(7-nitro-2-1,3-benzoxadiazol-4-yl)amino]lauroyl)-sn-gl-ycero-3-phosphocholine (16:0-12:0 NBD PC), cholesterol and mixtures/combinations thereof. In one embodiment, the MSNPs have an average diameter of less than about 200 nm. In one embodiment, the MSNPs have an average diameter of greater than about 20 nm. In one embodiment, the cargo comprises peptides, proteins, antibodies, nucleic acids, or drugs, e.g., drugs that inhibit ITA7 or expression thereof. In one embodiment, the nucleic acid cargo comprises RNA, e.g., siRNA, specific for ITA7. In one embodiment, the lipid bi- or multi-layer comprises DSPC, cholesterol, and PEG-DSPC. In one embodiment, the amount of DSPC is about 45 mol % to about 80 mol %. In one embodiment, the amount of DSPC is about 50 mol % to about 78 mol %. In one embodiment, the amount of cholesterol is about 10 mol % to about 50 mol %. In one embodiment, the amount of cholesterol is about 17 mol % to about 25 mol %. In one embodiment, the amount of PEG-DSPC is about 1 mol % to about 3 mol %. In one embodiment, the amount of PEG DSPC is about 2 mol % to about 2.7 mol %. Also provided is a pharmaceutical composition comprising a population of the protocells in combination with a pharmaceutically acceptable carrier, additive and/or excipient. Further provided is a method of preventing, inhibiting or treating a metabolic disease in a mammal such as a human, comprising administering to a mammal such as a human subject in need thereof, an effective amount of the pharmaceutical composition.

One embodiment provides a population of protocells comprising a lipid bi- or multi-layer, mesoporous silica nanoparticles (MSNPs), a cargo, and a targeting ligand comprising an antibody or fragment thereof that ITA7. Another embodiment provides a population of protocells comprising a lipid bi- or multi-layer, mesoporous silica nanoparticles (MSNPs) and a cargo, wherein the cargo comprises at least one of forskolin, β3-AR agonist CL-316243, rosiglitazone (Rosi) or GW0742 or peptides, proteins, antibodies, nucleic acids, or drugs that inhibit ITA7. In one embodiment, the MSNPs have a diameter ranging from about 1 nm to about 300 nm, is provided. In one embodiment, the targeting ligand is directed attached to the lipid layer. In one embodiment, the targeting ligand is indirectly attached to the lipid layer. In one embodiment, the targeting ligand is an antibody fragment or a scFv. In one embodiment, the lipid bi- or multi-layer is biotinylated. In one embodiment, the lipid bi or multi-layer comprises a thiolated PEG containing moiety. In one embodiment, the cargo comprises RNA.

In one embodiment, the lipid bi- or multi-layer comprises DSPC, cholesterol, PEG-DSPC, or a combination thereof. In one embodiment, the amount of DSPC is about 45 mol % to about 80 mol %. In one embodiment, the amount of DSPC is about 50 mol % to about 78 mol %. In one embodiment, the amount of cholesterol is about 10 mol % to about 50 mol %. In one embodiment, the amount of cholesterol is about 17 mol % to about 25 mol %. In one embodiment, the amount of PEG-DSPC is about 1 mol % to about 3 mol %. In one embodiment, the amount of PEG-DSPC is about 2 mol % to about 2.7 mol %. In one embodiment, the MSNPs have an average diameter of less than about 200 nm. In one embodiment, the MSNPs have an average diameter of greater than about 20 nm. In one embodiment.

Also provided is a pharmaceutical composition comprising the population of protocells, in combination with a pharmaceutically acceptable carrier, additive and/or excipient.

Further provided is a method of using the protocells, e.g., in a method of treating a metabolic disease. In one embodiment, the composition is intravenously administered. In one embodiment. In one embodiment, the targeting ligand is an antibody. In one embodiment, the cargo comprises siRNA specific for ITA7.

In still other embodiments, the disclosure includes methods of treatment and diagnostic methods which use the MSNPs and protocells described herein to treat and/or diagnose a variety of disorders, including metabolic disease/disorder, such as obesity.

The disclosure provides a population of protocells comprising a lipid bi- or multi-layer, mesoporous silica nanoparticles (MSNPs), a cargo, and an integrin alpha 7 (ITA7) targeting ligand. In one embodiment, the MSNPs have a diameter ranging from about 1 nm to about 300 nm. In one embodiment, the targeting ligand is an antibody. In one embodiment, the targeting ligand is an antibody fragment or a scFv. In one embodiment, the lipid layer is a bilayer. In one embodiment, the cargo comprises peptides, proteins, antibodies, nucleic acids, or drugs that inhibit ITA7. In one embodiment, the cargo comprises RNA In one embodiment, the RNA comprises siRNA. In one embodiment, the targeting ligand is non-covalently attached to the lipid layer. In one embodiment, the lipid layer comprises a lipid comprising biotin, biotin methyl ester, desthiobiotin, 2-iminobiotin, diamino biotin, biotin carbamate, or biotin carbonate. In one embodiment, the targeting ligand comprises avidin, streptavidin, neutravidin or bravavidin II. In one embodiment, the targeting molecule comprises biotin, biotin methyl ester, desthiobiotin, 2-iminobiotin, diamino biotin, biotin carbamate, or biotin carbonate. In one embodiment, the lipid layer comprises a lipid comprising avidin, streptavidin, neutravidin or bravavidin II. In one embodiment, the lipid bi- or multi-layer comprises DPPE, DSPC, cholesterol, PEG-DSPC, DODMA, DOTAP, DMPG; or a combination thereof. In one embodiment, the amount of DSPC is about 45 mol % to about 80 mol % or is about 50 mol % to about 78 mol %. In one embodiment, the amount of cholesterol is about 10 mol % to about 50 mol % or is about 17 mol % to about 25 mol %. In one embodiment, the lipid bi- or multilayer comprises: (a) at least one zwitterionic lipid selected from the group consisting of 1, 2-dioleoyl-sn-glycero-3-phosphocholine (DOPC), 1, 2-dioleoyl-sn-glycero-3-phosphoethanolamine (DOPE) and 1,2-distearoyl-sn-glycero-3-phosphocholine (DSPC); and (b) optionally, one or more additional electrically charged or neutral lipids selected from the group consisting of 1,2-dipalmitoyl-sn-glycero-3-phosphocholine (DPPC), dioleylglycero triethyleneglycyl iminodiacetic acid (DOIDA), distearylgtycerotdethyleneglycyl iminodiacetic acid (DSIDA), 1,2-dioleoyl-sn-glycero-3-[phosphorserine](DOPS), 1,2-dioleoyl-3-trimethylammonium-propane (18:1 DOTAP), 1,2-dioleoyl-sn-glycero-3-phospho-(1′-rac-glycerol) (DOPG). 1,2-dipalmitoyl-sn-glycero-3-phosphoethanolamine (DPPE). 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-Oleoyl-2-[12-[(7-nitro-2-1,3-benzoxadiazol-4-yl)amino]lauroyl]-sn-Glyce-ro-3-Phosphocholine (18:1-12:0 NBD PC), 1-palmitoyl-2-(12-[(7-nitro-2-1,3-benzoxadiazol-4-yl)amino]lauroyl)-sn-gl-ycero-3-phosphocholine (16:0-12:0 NBD PC), cholesterol and mixtures/combinations thereof. In one embodiment, the MSNPs are aminated with a composition selected from trimethoxysilyl propyl modified poly ethyleneimine (MW=1500-1800), (3-aminopropyl)triethoxysilane, (3-aminopropyl)trimethoxy silane, 3-aminopropylmethyl diethoxysilane, 3-aminopropyl dimethoxysilane, or mixtures thereof, or N trimethoxysilylpropyl-N,N,N-trimethyl ammonium chloride (TMAC-silane, MW 258), N1-(3-Trimethoxylsilylpropyl)diethylenetriamine, 3-(trimethoxysilyl)propyl-di-n-octylmethyl-ammonium chloride; 3-(trimethoxysilyl)propyl-n-octyldimethyl-ammonium chloride: 3-(trimethoxysilyl)propyl-di n-nonylmethyl-ammonium chloride; 3-(trimethoxysilyl)propyl-n-nonyl dimethyl-ammonium chloride; 3-(trimethoxysilyl)propyl-di-decylmethyl-ammonium chloride; 3-(trimethoxysilyl)propyl-di-n-undecylmethyl-ammonium chloride; 3-(trimethoxysilyl)propyl-n-undecyldimethyl-ammonium chloride; 3-(trimethoxysilyl)propyldi-n-dodecylmethyl-ammonium chloride; 3-(trimethoxysilyl)propyl-n-dodecyldimethyl-ammonium chloride; 3-(trimethoxysilyl)propyl-di-n-tridecyldimethyl-ammonium chloride; 3-(trimethoxysilyl)propyl-n-tridecyldimethyl-ammonium chloride; 3-(trimethoxysilyl)propyl-di-n-tetradecylmethyl-ammonium chloride; 3-(trimethoxysilyl)propyl-n-tetradecyldimethyl-ammonium chloride; 3-(triethoxysilyl)propyl-di-n-octylmethyl-ammonium chloride: 3-(triethoxysilyl)propyl-n-octyldimethyl-ammonium chloride; 3-(triethoxysilyl)propyl-di-n-nonylmethyl-ammonium chloride; 3(triethoxysilyl)propyl-n-nonyldimethyl-ammonium chloride; 3-(triethoxysilyl)propyl-di-n-decylmethyl-ammonium chloride; 3-(triethoxysilyl)propyl-n-decyldimethyl-ammonium chloride; 3-(triethoxysilyl)propyl-di-n-undecylmethyl-ammonium chloride; 3-(triethoxysilyl)propyl-n-undecyldimethyl-ammonium chloride; 3-(triethoxysilyl)propyl-di-n-dodecylmethvl-ammonium chloride; 3-(triethoxysilyl)propyl-n-dodecyldimethyl-ammonium chloride: 3-(triethoxysilyl)propyl-di-n-tridecylmethyl-ammonium chloride; 3-(triethoxysilyl)propyl-n-tridecyldimethyl-ammonium chloride; 3-(triethoxysilyl)propyl-di-n-tetradecylmethyl-ammonium chloride; 3-(triethoxysilyl)propyl-n-tetradecyldimethyl-ammonium chloride; 3-(tripropoxysilyl)propyl-di-n-octylmethyl-ammonium chloride: 3-(tripropoxysilyl)propyl-n-octyldimethyl-ammonium chloride; 3-(tripropoxysilyl)propyl-di-n-nonylmethyl-ammonium chloride; 3-(tripropoxysilyl)propyl-n-nonyldimethyl-ammonium chloride; 3-(tripropoxysilyl)propyl-di-n-decylmethyl-ammonium chloride; 3-(tripropoxysilyl)propyl-n-decyldimethyl-ammonium chloride: 3-(tripropoxysilyl)propyl-di-n-undecylmethyl-ammonium chloride: 3-(tripropoxysilyl)propyl-n-undecyldimethyl-ammonium chloride; 3-(tripropoxysilyl)propyl-di-n-dodecylmethyl-ammonium chloride: 3-(tripropoxysilyl)propyl-n-dodecyldimethyl-ammonium chloride; 3-(tripropoxysilyl)propyl-di-n-tridecylmethyl-ammonium chloride; 3-(tripropoxysilyl)propyl-n-tridecyldimethyl-ammonium chloride; 3-(tripropoxysilyl)propyl-di-n-tetradecylmethyl-ammonium chloride; 3-(tripropoxysilyl)propyl-n-tetradecyldimethyl-ammonium chloride; 3-(tributoxysilyl)propyl-di-n-octylmethyl-ammonium chloride; 3-(tributoxysilyl)propyl-n-octyldimethvl-ammonium chloride; 3-(tributoxysilyl)propyl-di-n-nonylmethyl-ammonium chloride: 3-(tributoxysilyl)propyl-n-nonyldimethyl-ammonium chloride; 3-(tributoxysilyl)propyl-di-n-decylmethyl-ammonium chloride; 3-(tributoxysilyl)propyl-n-decyldimethyl-ammonium chloride; 3-(tributoxysilyl)propyl-di-n-undecylmethvl-ammonium chloride; 3-(tributoxysilyl)propyl-n-undecyldimethyl-ammonium chloride: 3-(tributoxysilyl)propyl-di-n-dodecylmethyl-ammonium chloride; 3-(tributoxysilyl)propyl-n-dodecvldimethyl-ammonium chloride; 3-(tributoxysilyl)propyl-di-n-tridecylmethyl-ammonium chloride; 3-(tributoxysilyl)propyl-n-tridecyldimethyl-ammonium chloride; 3-(tributoxysilyl)propyl-di-n-tetradecylmethyl-ammonium chloride; 3-(tributoxysilyl)propyl-n-tetradecvldimethyl-ammonium chloride and mixtures thereof. In one embodiment, the MSNPs have an average diameter of less than about 200 nm. In one embodiment, the MSNPs have an average diameter of greater than about 20 nm. In one embodiment, the ITA7 targeting ligand comprises monoclonal antibody 334908 (ThermoFisher; Cat #MA5-23555) or an antigen binding fragment thereof, antibody ab203254 (abcam) or an antigen binding fragment thereof, E2 (e.g., E2 tag antibodies; LSBio) or an antigen binding fragment thereof, 8G2 or an antigen binding fragment thereof, PA2226 (Boster Bio) or an antigen binding fragment thereof, natalizumab (can be found under trade name TYSABRI®) or an antigen binding fragment thereof, vedolizumab (can be found under trade name ENTYVIOCR)) or an antigen binding fragment thereof, AMG-181 (Abrilumab, MedImmune) or an antigen binding fragment thereof, AJM300 or an antigen binding fragment thereof, Peptide X or an antigen binding fragment thereof or CDP323 or an antigen binding fragment thereof. In one embodiment, the cargo comprises RNA. In one embodiment, the RNA comprises siRNA. Further provides is a pharmaceutical composition comprising the population, in combination with a pharmaceutically acceptable carrier, additive and/or excipient. In one embodiment, the MSNPs have an average diameter ranging from about 100 nm to about 250 nm.

Further provided is a method to induce mammalian white adipose cells to mammalian brown-in-white cells. The method includes contacting mammalian white adipose cells with a composition comprising the population of protocells in an amount effective to produce mammalian brown-in-white cells.

Also provided is a method of treating a metabolic disease, comprising administering to a subject in need thereof an effective amount of a composition comprising the population of protocells. In one embodiment, the composition is systemically administered. In one embodiment, the composition is intravenously administered. In one embodiment, the targeting ligand is an antibody. In one embodiment, the targeting ligand is an antibody fragment or a scFv. In one embodiment, the cargo comprises peptides, proteins, antibodies, nucleic acids, or a drug. In one embodiment, the mammal is a human. In one embodiment, the mammal is obese. In one embodiment, the mammal has heart disease. In one embodiment, the mammal has type 2 diabetes. In one embodiment, the mammal has had a stroke or is at risk for a stroke.

### BRIEF DESCRIPTION OF FIGURES

FIG. 1. Overview of method.

FIGS. 2A-2D. Integrin alpha 7 (ITA7) expression profiles vary across adipose depots. Representative flow cytometry plot of ITA7 positive adipocytes in (A) Subcutaneous adipose tissue and (B) Brown adipose tissue (BAT). ~50% of adipocytes express ITA7 in white adipose tissue while almost none of adipocytes are positive for ITA7 in BAT. (C-D) Representative confocal microscopy images of subcutaneous adipose tissue showing how ITA7 surrounds some adipocytes but not all.

FIGS. 3A-3B. Human adipose derived stem cells (hADSC) were used to test uptake of targeted and untargeted protocells and the delivery of cargo. (A-B) Representative 3D confocal micrographs showing the different expression of ITA7 in hADSC after differentiation to beige adipocytes. (A) hADSC stained for ITA7 (yellow), Bodipy (green) to label adipocytes and DAPI (blue) to label nuclei. (B) Same image without the ITA7 staining.

FIG. 4. Integrins and extracellular matrix proteins modulate adipocyte thermogenic capacity.

FIG. 5. Strategies for protocell design.

FIGS. 6A-6D. MSN and liposomes do not affect the process of lipid loading but PEI significantly decrease their formation. (A) hADSC adipocytes (A) after treated with MSN-PEI or (B) untreated. (C) The amount of Bodipy fluorescence was significantly decrease in cells treated with MSN-PEI or protocells including PEI. (D) Number of cells was unaffected by any treatment.

FIG. 7. Characteristics of different protocells. The cytotoxicity of the different protocells components into adipocytes was examined by looking at the number of cells as well as how those components affect the lipid loading (formation of adipocytes).

FIG. 8. Adding ITA7 targeting ligand on the protocell surface resulted in a higher uptake of siRNA-laden protocells by adipocytes.

FIGS. 9A-9D. Differentiated beige adipocytes showed a greater uptake of ITA7-protocells compared to untargeted protocells based on fluorescence measurements. 2D images of hADSC after 24 h of treatment with (A) ITA7-protocells and (B) protocells. (C) Quantification by plate reader of protocells fluorescence after 24 h of treatment in 24 wells, showed a significant increase of ITA7-protocell fluorescence in cells. (D) Adipocytes with fluorescent protocells after trypsinization illustrate the major uptake of ITA7-protocells in adipocytes compared to untargeted protocells.

FIGS. 10A-10C. Flow cytometry scatter plots: side scattering (SSC-A) versus fluorescence intensity of beige adipocytes (differentiated hADSC) A.) untreated. B.) treated with untargeted protocells for 24 h, C.) hADSC exposed to ITA7-protocells for 24 h. 20 μg/ml of protocells was used for all exposed samples.

FIG. 11. Target ITA7 gene expression following two doses of treatment with ITA7 siRNA using targeted and untargeted protocells. Lipofectamine was used as a positive control. ITA7 gene expression was measured by RT-PCR.

FIGS. 12A-12D. LCMSN-FSK synthesis and characterization. (A) Schematic showing the step-by-step formation of LCMSN-FSK (B) TEM images of implemented MSN (C) Hydrodynamic size, polydispersity index and zeta potential evolution of pure liposomes, MSNs and their corresponding FSK-loaded constructs during formation. (D)TGA curves of different samples providing the FSK %.

FIGS. 13A-13E. Differentiation of hASC to mature adipocytes. (A-B) Representative phase microscopy image of (A) undifferentiated hASC and (B) differentiated mature adipocytes respectively. (C) Fluorescent image counterstained with BODIPY, of hASCs after 14 days of differentiation. (D) mRNAs levels of adiponectin and PPARγ were evaluated in adipocytes relative to hASC at d0 (undifferentiated cells). Scale bar=100 μm. Values are means±standard errors, from one-way ANOVA analysis followed with Tukey's multiple comparisons test. **, p<0.01; ****, p<0.0001; n=3 biological samples with 3 replicates per group.

FIGS. 14A-14F. Analysis of LCMSNs uptake in mature adipocytes in vitro and ex vivo. (A) Fluorescence measurement of adipocytes in a microplate reader after treatment of rhodamine labeled-LCMSN. (B) Brightfield microscopy image of mature adipocytes after 24 h of LCMSN treatment, demonstrated presence of rhodamine fluorescence LCMSN in adipocytes and not outside the cells. (C) Flow cytometry of adipocytes treated with cy3 labeled LCMSNs at 3 h, 6 h, 24 h and 48 h. (D) Representative confocal image and analysis of mature adipocytes with rhodamine labeled LCMSNs in between lipid droplets, confirming intracellular presence of nanoparticles. (E) At least 30 adipocytes were evaluated per time point to quantify fluorescence intensity inside the adipocyte, specified by a selected ROI. (F) Ex vivo representative confocal microscopy image of adipose tissue after mice treatment with fluorescently labeled LCMSN for 24 h. LCMSN (red) are observed in closed proximity of lipid droplets and in the adipocyte nuclei counterstained with DAPI (dark blue). (G) Schematic of what it is described and observed in confocal images. For in vitro assays, data are shown as the mean from 6 different experiments. Two different mice were used for ex vivo treatment of adipose tissue.

FIGS. 15A-15D. In vitro efficacy studies in mature human adipocytes treated with either LCMSNs loaded with forskolin (LCMSN-FSK), or forskolin alone (FSK) compared to control cells, for different incubation time points: (A-B) Gene expression levels of thermogenic marker UCP1 and Cox7A1, (C) Glucose uptake and (D) lipolysis efficacy. Values are means±standard errors, from one-way ANOVA analysis followed with Tukey's multiple comparisons test. *, p<0.05; **, p<0.01; ***, p<0.001; ****, p<0.0001: n=3 biological samples with 3 replicates per group.

FIGS. 16A-16F. Oxygen consumption rate (OCR) trace of human adipocytes after different treatments, was determined using a Seahorse XF96 Analyzer. (B) Basal respiration, (C) proton leak, (D) ATP production, (E) maximal respiration, and (F) spare respiratory capacity were calculated. Results are reported as mean±standard error of 3 replicates. *=p<0.05, **=p<0.01, ***=p<0.001, and ****=p<0.0001.

FIGS. 17A-17D. Biodistribution after in vivo subcutaneous injection of DyLight 633 labeled LCMSNs-FSK. (A) KINO images of mice. (B) ex vivo imaging of organs at t=24 h and C) t=48 h subcutaneous administration of LCMSNs compared to CTL. (C) Fluorescence of iWAT compared to other organs after 24 h injection. (D) Fluorescence intensity % of iWAT at different time points post subcutaneous injection compared to other harvested organs. Images are representatives of three independent experiments.

FIGS. 18A-18E. Thermogenic gene markers analysis, and adipose tissue lipolysis measurements after in vivo subcutaneous injection of LCMSNs-FSK. (D-E) Gene expression of UCP1 and Cox7A1, and (F) Lipolysis activity in iWAT at different time points post treatment injection. (G) Protein expression of UCP1 in iWAT after 24 h treatment injection was assessed by western blot. (H) UCP1 band intensity was quantified and normalized to total actin signal. n=2 animals. Data are presented as mean±standard error of the mean with *. p<0.05: **. p<0.01; ***, p<0.001: ****, p<0.0001 as significant from one-way ANOVA followed with Tukey's multiple comparison.

FIGS. 19A-19C. LCMSNs-FSK prevention of obesity in DIO C57BL/6J mice. (A) Schematic showing the injection treatment frequency and the equivalent forskolin dose administered (B) Mice food intake. (C) Percentage body weight increase of mice over the six-weeks. n=3 animals per group. Data are presented as mean±standard error of the mean with *p<0.05, **p<0.01, ***p<0.001 as significant from one-way ANOVA followed with Tukey's multiple comparisons test.

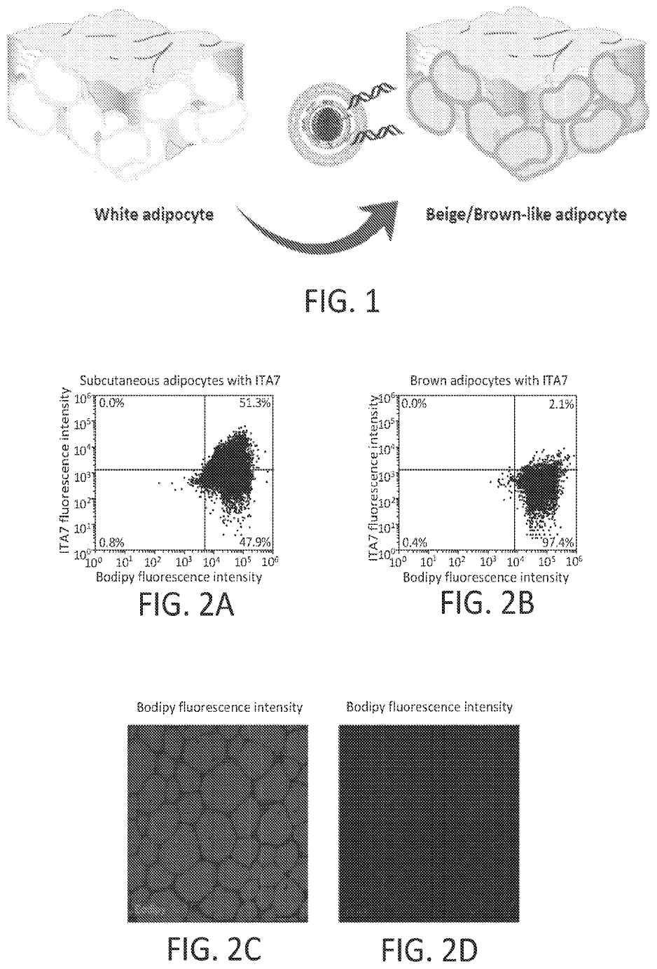

### DETAILED DESCRIPTION

The following terms shall be used throughout the specification to describe the present disclosure. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the methods and materials are now described.

It must be noted that as used herein and in the appended claims, the singular forms “a,” “and” and “the” include plural references unless the context clearly dictates otherwise.

The term “patient” or “subject” is used throughout the specification within context to describe an animal, generally a mammal, especially including a domesticated animal or a human, to whom treatment, including prophylactic treatment (prophylaxis), with the compounds or compositions according to the present disclosure is provided. For treatment of those infections, conditions or disease states which are specific for a specific animal such as a human patient, the term patient refers to that specific animal. In most instances, the patient or subject of the present disclosure is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts (including alternative pharmaceutically acceptable salts when a pharmaceutically acceptable salt is disclosed) and prodrug forms. The term compound herein refers to stable compounds. Within its use in context, the term compound may refer to a single compound or a mixture of compounds as otherwise described herein. One or more bioactive agent (any agent which produces an intended biological, including pharmacological effect) may be included in MSNPs according to the present disclosure to provide pharmaceutical compositions hereunder.

The term “mesoporous silica nanoparticles” (MSNPs) is used to describe nanoparticles according to the present disclosure which are modified to target specific cells in vivo for diagnostic and/or therapeutic purposes.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multi-particulate (e.g., a porous nanoparticulate) means that at least 50% of the particles therein are of a specified size. Accordingly, “effective average particle size of less than about 2,000 nm in diameter” means that at least 50% of the particles therein are less than about 2000 nm in diameter. In certain embodiments, nanoparticles have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. In certain aspects of the present disclosure, where administration via intravenous, intramuscular, intraperitoneal, retro-orbital and subcutaneous injection routes produces long residence times (on the order of at least 12 hours to 2 weeks or more) and greater biodistribution and/or bioavailability, the MSNPs and protocells are monodisperse and generally no greater than about 50 nm in average diameter, often less than about 30 nm in average diameter, as otherwise described herein. The term “D₅₀” refers to the particle size below which 50% of the particles in a multi-particulate fall. Similarly, the term “D₉₀” refers to the particle size below which 90% of the particles in a multi-particulate fall.

The MSNP size distribution depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, mesoporous silica nanoparticles can range, e.g., from around 1 nm to around 500 nm in size, including all integers and ranges there between. The size is measured as the longest axis of the particle. In various embodiments, the particles are from around 5 nm to around 500 nm and from around 10 nm to around 100 nm in size. The mesoporous silica nanoparticles have a porous structure. The pores can be from around 0.5 nm to about 25 nm in diameter, often about 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, MSNPs according to the present disclosure are monodisperse and range in size from about 25 nm to about 300 nm; exhibit stability (colloidal stability); have single cell binding specification to the substantial exclusion of non-targeted cells; are neutral or cationic for specific targeting (e.g., cationic); are optionally modified with agents such as PEI, NMe3+, dye, crosslinker, ligands (ligands provide neutral charge); and optionally, are used in combination with a cargo to be delivered to a targeted cell.

In certain alternative embodiments, the MSNPs are monodisperse and range in size from about 25 nm to about 300 nm. The sizes used may include 50 nm (+/−10 nm) and 150 nm (+/−15 nm), within anarrow monodisperse range, but may be narrower in range. A broad range of particles is not used because such a population is difficult to control and to target specifically.

In certain alternative embodiments, the present disclosure is directed to MSNPs and for example, protocells of a particular size (diameter) ranging from about 0.5 to about 30 nm, about 1 nm to about 30 nm, often about 5 nm to about 25 nm (e.g., less than about 25 nm), often about 10 to about 20 nm, for administration via intravenous, intramuscular, intraperitoneal, retro-orbital and subcutaneous injection routes. These MSNPs and/or protocells are often monodisperse and provide colloidally stable compositions. These compositions can be used to target tissues in a patient or subject because of enhanced biodistribution/bioavailability of these compositions, and optionally, specific cells, with a wide variety of therapeutic and/or diagnostic agents which exhibit varying release rates at the site of activity. As noted in the present application, MSNPs and protocells may have a charged surface (zeta potential) which ranges from about −40 EV to +40 EV. MSNPs and protocells according to the present disclosure may exhibit varied surface charges as a consequence of the componentry used to create the MSNPs. A typical MSNP based upon silica (without amine modification) exhibits a negatively charged surface having a zeta potential often within the range of about −10 EV to about −40 EV. A negative surface charge, or alternatively, a positive surface charge which is presented through use of quaternary amines for MSNPs and protocells pursuant to the present disclosure are consistent with these particles being less interactive with vascular/endothelial tissue and providing greater distribution to and high residence times in tissue after administration. A positively charged MSNP exhibits a zeta potential of about +10 EV to about +40 EV. A positive surface charge for MSNPs and protocells pursuant to the present disclosure, especially from primary amines, and to a less extent secondary and tertiary amines, are consistent with these particles being more interactive with vascular tissue and providing limited distribution principally to vascular tissue after administration. It is noted that when secondary and tertiary amines, as opposed to primary amines, are used to provide a more positively charged surface, whether the actual surface charge is negative or positive, these may exhibit non-specific binding to vascular tissue (endothelial tissue), but the effect is substantially less (muted) than the effect is for primary amines. Of course, using mixtures of amines may be used to influence both the surface charge (zeta potential) as well as the non-specific binding of the nanoparticles to vascular tissue along a continuum from very little, if any binding (quaternary amines) to some binding (tertiary and secondary amines) to specific targeting of endothelial cells utilizing primary amines.

The term “uniform surface” is used to describe a surface which contains a uniform surface charge. Uniform surfaces occur for MSNPs (e.g., PEGylated) which contain quaternary amines such as the charge is consistently projected on the whole surface of the MSNP without appreciable patches or gaps in the surface charge. A “non-uniform surface” describes a surface of an MSNP which contains patches of charge which are distinguishable from the broader portions of the surface. In the case of MSNPs which are modified with primary amines, the overall surface may be neutral or charged, but the primary amine creates a patch of more positive charge with protruding protonated amines characterizing the patches on the surface of the MSNPs. The surface of the MSNPs, including protocells according to the present disclosure may be measured and/or identified using cryo-TEM and TEM analysis, among others. These analyses look at the characteristics of the binding of a metal with high electron density—often a heavy metal such as gold, silver, iron and the like- to produce a 3-dimensional spatial arrangement on the nanoparticle. Uniform surfaces tend to be consistent and uniform in their surface charge, whereas non-uniform surfaces tend to have areas of concentrated charge in a patchwork that can often be random.

The term “PEGylated” in its principal use refers to an MSNP which has been produced using PEG-containing silanes or zwitterionic group-containing silanes to form the MSNP. In general, the amount of the PEG-containing silanes and/or zwitterionic-containing silanes which optionally are used to produce MSNPs according to the present disclosure represent about 0.05% to about 50% (about 0.1% to about 35%, about 0.5% to about 25%, about 1% to about 20%, about 2.5% to about 30%, about 0.25% to about 10%, about 0.75% to about 15%) by weight of these monomers in combination with the silane monomers which are typically used to form MSNPs. A PEG-containing silane is any silane which contains a PEG as one of the substituents and the remaining groups can facilitate the silane reacting with other silanes to produce MSNPs according to the present disclosure. PEG-containing silanes and/or zwitterionic-containing silanes which may be used in the present disclosure to create PEGylated MSNPs include 2-[methoxy(polyethyleneoxy)propyl]trimethoxysilane (containing varying molecular weights of PEG ranging from about 100 to 10,000 average molecule weight, often about 200 to 5,000 average molecular weight, about 1,000-2,500 average molecular weight, about 1500-2000 average molecular weight) and 3-([Dimethoxyl(3-trimethoxysilyl)propyl]ammonio)propane-1-sulfonate and mixtures thereof, among others. The term “PEGylated” may also refer to lipid bilayers which contain a portion of lipids which are PEGylated (from about 0.02% up to about 50%, about 0.1% to about 35%, about 0.5% to about 25%, about 1% to about 15%, about 0.5% to about 7.5%, about 1% to about 12.5% by weight of the lipids used to form the lipid bilayer or multilayer). These lipids often are amine-containing lipids (e.g., DOPE and DPPE) which are conjugated or derivatized to contain a PEG group (having an average molecule weight ranging from about 100 to 10,000, about 200 to 5,000, about 1,000-5,000, including 1,000, 2000, 3000 and 3400) and combined with other lipids to form the bilayer/multilayer which encapsulates the MSNP.

The term “non-specific binding” refers to the binding which occurs between a charged surface of the MSNPs according to the present disclosure and endothelial tissue pursuant to the present disclosure because the interaction between the particles and the tissue surface are based non-specifically upon the interactions of charges on the particles and the tissue surface rather than a ligand-ligand interaction. In the case of “specific binding” the interaction between the particle and a target is based upon a specific ligand-ligand interaction. It is noted that when a particle exhibits low non-specific binding, that particle may exhibit very little binding (i.e., little specific or non-specific binding) or more specific binding (greater ligand-ligand interaction) depending upon the context of its use.

The terms “targeting ligand” and “targeting active species” are used to describe a compound or moiety (e.g., an antigen) which is complexed or covalently bonded to the surface of a MSNPs and/or protocells according to the present disclosure which binds to a moiety on the surface of a cell to be targeted so that the MSNPs and/or protocells may selectively bind to the surface of the targeted cell and deposit their contents into the cell. The targeting active species for use in the present disclosure may be a targeting peptide as otherwise described herein, a polypeptide including an antibody or antibody fragment, an aptamer, or a carbohydrate, among other species which bind to a targeted cell.

Ligands which may be used to target cells include peptides, affibodies and antibodies (including monoclonal and/or polyclonal antibodies). In certain embodiments, targeting ligands selected from the group consisting of Fc gamma from human IgG (which binds to Fcgamma receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells). Other targeting peptides known in the art may also be used.

The charge of the nanoparticle is controlled based on what is to be accomplished (via PEI, NMe³⁺, dye, crosslinker, ligands, etc.), but for targeting vascular tissue the charge may be cationic. In the case of enhanced biodistribution, the charge may be anionic, but may be cationic provided that the charge occurs principally from the inclusion of quaternary amines. Charge also changes throughout the process of formation. Initially, in certain embodiments the targeted particles are cationic and are often delivered as cationically charged nanoparticles, however post modification with ligands they are closer to neutral. The ligands which find use in the present disclosure include peptides, affibodies and antibodies, among others. These ligands are site specific and are useful for targeting specific cells which express peptides to which the ligand may bind selectively to targeted cells.

MSNPs pursuant to the present disclosure may be used to deliver cargo to a targeted cell, including, for example, cargo component selected from the group consisting of a polynucleotide such as DNA, including double stranded linear DNA or a plasmid DNA, RNA, including small interfering RNA, small hairpin RNA, microRNA, siRNA, a drug, an imaging agent, or a mixture thereof.

In protocells of the disclosure, a biotinylated or PEGylated lipid bi- or multilayer encapsulates a population of MSNPs as described herein and comprises (1) biotin or an analog thereof or an optionally-thiolated PEG (2) at least one lipid and, optionally (3) at least one targeting ligand which is conjugated to the outer surface of the lipid bi- or multilayer.

Protocells of the disclosure are highly flexible and modular. High concentrations of physiochemically-disparate molecules can be loaded into the protocells and their therapeutic and/or diagnostic agent release rates can be optimized without altering the protocell's size, size distribution, stability, or synthesis strategy. Properties of the supported lipid bi- or multilayer and mesoporous silica nanoparticle core can also be modulated independently, thereby optimizing properties as surface charge, colloidal stability, and targeting specificity independently from overall size, type of cargo(s), loading capacity, and release rate.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, delay in or inhibition of the likelihood of the onset of the disease, etc. Treatment can also be used to provide prevention (prophylaxis/reducing the likelihood) of a disease state occurring, but the present disclosure contemplates a distinction between the treatment of a disease state and/or condition and the prevention (prophylaxis/reducing the likelihood) that a disease state or condition will occur, within the context of such treatment/prevention.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

Treatment, as used herein, may encompass prophylactic and/or therapeutic treatment depending on context, principally of metabolic disease, but also of other disease states. Compounds according to the present disclosure can, for example, be administered prophylactically to a mammal in advance of the occurrence of disease to reduce the likelihood of that disease. Prophylactic administration is effective to reduce or decrease the likelihood of the subsequent occurrence of disease in the mammal or decrease the severity of disease (inhibition) that subsequently occurs. Alternatively, compounds according to the present disclosure can, for example, be administered therapeutically to a mammal that is already afflicted by disease. In one embodiment of therapeutic administration, administration of the present compounds is effective to eliminate the disease.

The compounds/bioactive agents may also be included in MSNPs, including protocells, having average diameters which are less than about 50 nm, or less than 30 nm for formulating compositions adapted for intravenous, intramuscular, intraperitoneal, retro-orbital and subcutaneous injection routes.

Typically the MSNPs and protocells according to the present disclosure are loaded with cargo to a capacity up to about 50 weight % or more (from about 0.01% to about 50%, about 0.02% to about 40%, about 0.2 to about 35%, about 0.5% to about 25%, about 1% to about 25%, about 1.5% to about 15%, about 0.1% to about 10%, about 0.01% to about 5%): defined as (cargo weight/weight of loaded protocell).times.100. The optimal loading of cargo is often about 0.01 to 10% but this depends on the drug or drug combination which is incorporated as cargo into the MSNPs. This is generally expressed in μM per 10¹⁰ particles where we have values ranging from 2000-100 μM per 10¹⁰ particles. MSNPs according to the present disclosure may exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the MSNPs according to one embodiment of the present disclosure, the surface area is mainly internal as opposed to the external geometric surface area of the nanoparticle.

The term “lipid” is used to describe the components which are used to form lipid bi- or multilayers on the surface of the nanoparticles which are used in the present disclosure. Various embodiments provide nanostructures which are constructed from nanoparticles which support a lipid bilayer(s). In embodiments according to the present disclosure, the nanostructures may include, for example, a core-shell structure including a porous particle core surrounded by a shell of lipid bilayer(s). The nanostructure, e.g., a porous alum nanostructure as described above, supports the lipid bilayer membrane structure.

The lipid bi- or multilayer supported on the porous particle according to one embodiment of the present disclosure has a lower melting transition temperature, i.e., is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

In the present disclosure, the lipid bi- or multilayer may vary significantly in composition. Ordinarily, any lipid or polymer which may be used in liposomes may also be used in MSNPs according to the present disclosure. Lipids are as otherwise described herein.

In embodiments according to the disclosure, the lipid bi- or multilayer of the protocells can provide biocompatibility and can be modified to possess targeting species including, for example, antigens, targeting peptides, fusogenic peptides, antibodies, aptamers, and PEG (polyethylene glycol) to allow, for example, further stability of the protocells and/or a targeted delivery into a cell to maximize an immunogenic response. PEG, when included in lipid bilayers (using PEGylated lipids), can vary widely in molecular weight (although PEG ranging from about 10 to about 100 units of ethylene glycol, about 15 to about 50 units, about 15 to about 20 units, about 15 to about 25 units, about 16 to about 18 units, etc., may be used) and the PEG component which is generally conjugated to a phospholipid through an amine group comprises about 1% to about 20%, e.g., about 5% to about 15%, about 10% by weight of the lipids which are included in the lipid bi- or multilayer.

Numerous lipids which are used in liposome delivery systems may be used to form the lipid bi- or multilayer on nanoparticles to provide MSNPS according to the present disclosure. Virtually any lipid which is used to form a liposome may be used in the lipid bi- or multilayer which surrounds the nanoparticles to form MSNPS according to an embodiment of the present disclosure. Lipids for use in the present disclosure include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000](18:1 PEG-2000 PE), 1,2-dipalmitoyl-sn-glycero-3-phosphoethanolamine-N-[methoxy(polyethylene glycol)-2000](16:0 PEG-2000 PE), 1-Oleoyl-2-[12-[(7-nitro-2-1,3-benzoxadiazol-4-yl)amino]lauroyl]-sn-Glyce-ro-3-Phosphocholine (18:1-12:0 NBD PC), 1-palmitoyl-2-(12-[(7-nitro-2-1,3-benzoxadiazol-4-yl)amino]lauroyl)-sn-gl-ycero-3-phosphocholine (16:0-12:0 NBD PC), cholesterol and mixtures/combinations thereof. Cholesterol, not technically a lipid, but presented as a lipid for purposes of an embodiment of the present disclosure given the fact that cholesterol may be an important component of the lipid bilayer of protocells according to an embodiment of the disclosure. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including immunogenic peptides, proteins and antibodies, RNA and DNA through the amine group on the lipid.

MSNPs and protocells of the disclosure may be PEGylated with a variety of polyethylene glycol-containing compositions. PEG molecules can have a variety of lengths and molecular weights and include, but are not limited to, PEG 200, PEG 1000, PEG 1500, PEG 4600, PEG 10,000, PEG-peptide conjugates, or combinations thereof. Example 3 herein describes the use of 2-[methoxy(polyethyleneoxy)-propyl]trimethoxysilane (MW 550-750, 9-12 EO, PEG-silane) for MSNP PEGylation. Typically, pegylation occurs by using a silyl agent containing a PEG groups (PEG-silane) which is added to the silane mixture in synthesizing MSNPs according to the present disclosure. Alternatively, a reactive amine group on the surface of the MSNPs may be functionalized by reacting the amine with a PEG containing group to form a PEG group on the amine.

The term “reporter” is used to describe an imaging agent or moiety which is incorporated into the phospholipid bilayer or cargo of MSNPS according to an embodiment of the present disclosure and provides a signal which can be measured. The moiety may provide a fluorescent signal or may be a radioisotope which allows radiation detection, among others. Exemplary fluorescent labels for use in MSNPs and protocells (e.g., via conjugation or adsorption to the lipid bi- or multilayer or silica core, although these labels may also be incorporated into cargo elements such as DNA, RNA, polypeptides and small molecules which are delivered to cells by the protocells) include Hoechst 33342 (350/461), 4′,6-diamidino-2-phenylindole (DAPI, 356/451). Alexa Fluor® 405 carboxylic acid, succinimidyl ester (401/421). CellTracker™. Violet BMQC (415/516). CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX©. Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666). Alexa Fluor® 647 carboxylic acid, succinimidyl ester (650/668), Ulysis™ AlexaFluor® 647 Nucleic Acid Labeling Kit (650/670) and AlexaFluor® 647 conjugate of annexin V (650/665). Moities which enhance the fluorescent signal or slow the fluorescent fading may also be incorporated and include SlowFade®. Gold antifade reagent (with and without DAPI) and Image-iT® FX signal enhancer. All of these are well known in the art.

Additional reporters include polypeptide reporters which may be expressed by plasmids (such as histone-packaged supercoiled DNA plasmids) and include polypeptide reporters such as fluorescent green protein and fluorescent red protein. Reporters pursuant to the present disclosure are utilized principally in diagnostic applications or the progress of therapy in a patient or subject.

Pharmaceutical compositions according to the present disclosure comprise an effective population of MSNPs and/or protocells as otherwise described herein formulated to affect an intended result (e.g., immunogenic result, therapeutic result and/or diagnostic analysis, including the monitoring of therapy) formulated in combination with a pharmaceutically acceptable carrier, additive or excipient. The MSNPs and/or protocells within the population of the composition may be the same or different depending upon the desired result to be obtained. Pharmaceutical compositions according to the present disclosure may also comprise an addition bioactive agent or drug.

Generally, dosages and routes of administration of the compound are determined according to the size and condition of the subject, according to standard pharmaceutical practices. Dose levels employed can vary widely and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the disclosure include humans, companion animals, laboratory animals, and the like. The disclosure contemplates immediate and/or sustained/controlled release compositions, including compositions which comprise both immediate and sustained release formulations. This is particularly true when different populations of MSNPs and/or protocells are used in the pharmaceutical compositions or when additional bioactive agent(s) are used in combination with one or more populations of protocells as otherwise described herein.

In certain formulation embodiments of the disclosure include protocells comprised of mesoporous silica nanoparticulates (MSNPs) that (a) are loaded with one or more pharmaceutically-active agents and (b) that are encapsulated by and that support a lipid bilayer, and wherein the protocell has an average diameter of between about 1 nm to about 50 nm, e.g., between about 1 nm to about 30 nm, about 5 nm to about 25 nm, often 10 nm to about 25 nm, about 10 to about 20 nm. It has unexpectedly been discovered that the administration of protocells comprising therapeutic and/or diagnostic agents via intravenous, intramuscular, intraperitoneal, retro-orbital and especially subcutaneous routes of administration at the average diameters indicated above provide enhanced biodistribution, enhanced bioavailability and increased residence time (often at least 12-24 hours to several days up to a week or in certain cases, two weeks to a month or even longer), of these protocells compared to protocells with average diameters which are in excess of 50 nm, often greater than about 100 nm or more (e.g., 200-300 nm). Accordingly, the present compositions and methods of treatment and diagnosis in these routes of administration are greatly facilitated compared to compositions which contain protocells of larger diameters. Compositions according to the present disclosure may be used to administer cargo as otherwise described herein to a patient or subject through intravenous, intramuscular, intraperitoneal, retro-orbital and subcutaneous routes of administration, with unexpected biodistribution, bioavailability and residence times far exceeding compositions utilizing nanoparticles with average diameters in excess of 50-100 nm or greater (200-250 nm).

Formulations containing the compounds according to the present disclosure may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions according to the present disclosure typically include a conventional pharmaceutical carrier or excipient and may additionally include other medicinal agents, carriers, adjuvants, additives and the like. In some embodiments, the composition is about 0.1% to about 85%, about 0.5% to about 75% by weight of a compound or compounds of the disclosure, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion. Liquid compositions can be prepared by dissolving or dispersing the population of MSNPs and/or protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present disclosure.

Methods of treating patients or subjects in need for a particular disease state or infection comprise administration an effective amount of a pharmaceutical composition comprising therapeutic MSNPs and/or protocells and optionally at least one additional bioactive (e.g., antiviral) agent according to the present disclosure.

Diagnostic methods according to the present disclosure comprise administering to a patient in need an effective amount of a population of diagnostic MSNPs and/or protocells (e.g., MSNPs and/or protocells which comprise a target species, such as a targeting peptide which binds selectively to certain adipocyte cells and a reporter component to indicate the binding of the protocells whereupon the binding of protocells to cells as evidenced by the reporter component (moiety) will enable a diagnosis of the existence of a disease state in the patient.

An alternative of the diagnostic method of the present disclosure can be used to monitor the therapy of a disease state in a patient, the method comprising administering an effective population of diagnostic MSNPs and/or protocells (e.g., MSNPs and/or protocells which comprise a target species, such as a targeting peptide which binds selectively to target cells and a reporter component to indicate the binding of the protocells to cells in a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

The present disclosure also is directed to a process or processes for preparing the MSNPs according to the present disclosure.

In one embodiment, the disclosure is directed to a process for making a population of monodisperse mesoporous silica nanoparticles (MSNPs) that exhibit a relatively non-uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60, 55, 50, 45, 40, 35 or 30 nm (e.g., less than 50 nm, or less than 30, 25, 20, 15 or 10 nm)), a pore size of between about 1 nm to about 200 nm or between about 50, 40, 30, 25, 20, 15, 10, 9, 8, 7, 6, 5, 4, 3, 2 or 1 nm, a surface area of between about 100-1,000 m²/g, and a Zeta potential (.zeta.) of between about −40 mV to about +40 mV (e.g., greater than 0 mV) and that, upon administration in vivo, exhibit non-specific binding to white blood cells and arterial, venous and/or capillary vessels or combinations thereof, the process comprising:

(a) (1) preparing a mesoporous silica colloidal solution comprising: (1) a solvent solution comprising: (i) an alkoxysilane selected from the group consisting of tetramethylortho silicate (TMOS), tetraethylortho silicate (TEOS), tetrakis(2-hydroxyethyl)ortho silicate (THEOS), methyldiethoxysilane (MDES), 3-(glycidoxypropyl)triethoxysilane (GPTMS), 3-(trimethyoxysilyl)propylacrylate (TMSPA), N-(3-triethoxysilylpropyl)pynole (TESPP), vinyltriethyoxysilane (VTES), methacryloxypropyltriethoxysilane (TESPM), diglycerylsilane (DGS), methyltriethoxysilane (MTMOS), trimethylmethoxvsilane (TMMS), ethyltriethoxysilane (TEES), n-propyltriethoxysilane (TEPS), n-butyltriethyoxysilane (TEBS), 3-aminopropyltriethoxysilane (APTS), 2-(2,4-dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), and triethoxysilyl-terminated poly(oxypropylene) (ii) a solvent (iii) optionally, a reporter, and (2) a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethylammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., e.g., at about 50° C.; (b) including in said mesoporous silica colloidal solution a composition comprising a primary amine group and, optionally, a PEG-silane compound to produce a nanoparticle containing amine groups; and (c) hydrothermally treating the aminated nanoparticles produced in step (b) by heating the nanoparticles at a temperature of between about 100° C. to about 150° C. (e.g., about between about 110° C. to about 140° C., e.g., between about 115° C. to about 135° C., e.g., at about 120° C.) to yield the optionally PEGylated, monodisperse mesoporous silica nanoparticles (MSNPs); wherein the process can be one pot or in steps.

In the above process, (a) the alkoxysilane may be 3-aminopropyltriethoxysilane (APTS), the solvent may be N, N-dimethyl formamide (DMF) and the reporter may be rhodamine B isothiocynate (RITC); and the composition comprising a primary amine group is trimethoxysilylpropyl modified polyethyleneimine (50% in isopropanol, M.W. 1500-1800, PEI-silane) and the PEG-silane compound is methoxy(polyethyleneoxy)propyl]trimethoxysilane (Mw 550-750, 9-12 EO, PEG-silane).

In an alternative embodiment, the present disclosure is directed to a process for making a population of monodisperse mesoporous silica nanoparticles (MSNPs) that exhibit a relatively uniform surface charge distribution and colloidal stability and that have a diameter ranging from about 25 nm to about 300 nm (or from about 25 nm to about 200 nm, or from about 25 nm to about 100 nm, or from about 25 nm to about 95, 90, 85, 80, 75, 70, 65, 60, 55, 50, 45, 40, 35 or 30 nm (e.g., less than 50 nm, such as less than 30, 25, 20, 15 or 10 nm)), a pore size of between about 1 nm to about 200 nm or between about 50, 40, 30, 25, 20, 15, 10, 9, 8, 7, 6, 5, 4, 3, 2 or 1 nm, a surface area of between about 100-1,000 m²/g, and a Zeta potential (Z; ζ) of between about −40 mV to about +40 mV (e.g., less than 0 mV) and that, upon administration in vivo, exhibit minimal non-specific binding and prolonged circulation, the process comprising:

(a) (1) preparing a mesoporous silica colloidal solution comprising: (1) a solvent solution comprising: (i) an alkoxysilane selected from the group consisting of tetramethylortho silicate (TMOS), tetraethvlortho silicate (TEOS), tetrakis(2-hydroxyethyl)ortho silicate (THEOS), methyldiethoxysilane (MDES), 3-(glycidoxvpropvl)triethoxysilane (GPTMS), 3-(trimethyoxysilyl)propylacrylate (TMSPA), N-(3-triethoxysilylpropyl)pyrrole (TESPP), vinyltriethyoxysilane (VTES), methacryloxypropyltriethoxysilane (TESPM), diglycervlsilane (DGS), methyltriethoxysilane (MTMOS), trimethylmethoxysilane (TMMS), ethyltriethoxysilane (TEES), n-propyltriethoxysilane (TEPS), n-butyltriethyoxysilane (TEBS), 3-aminopropyltriethoxysilane (APTS), dinitrophenylamino)propyltriethoxysilane, mercaptopropyltriethoxysilane (TEPMS), 2-(3-aminoethylamino)propyltriethoxysilane, isocyanatopropyltriethoxysilane, hydroxyl-terminated polydimethylsiloxane, triethoxysilyl-terminated polydimethylsiloxane, methyltriethoxysilane (MTES), and triethoxysilyl-terminated poly(oxypropylene) (ii) a solvent (iii) optionally, a reporter, and (2) a surfactant which is selected from the group consisting of polyvinyl alcohol (PVA), dioctyl sodium sulfosuccinate, methyl cellulose, polysorbates, cetyltrimethylammonium bromide (CTAB), dodecylamine (DDA), 1,2-di-O-octadecenyl-3-trimethylammonium propane (DOTMA), and 1,2-Dioleoyl-3-trimethvlammonium-propane (DOTAP) and which is heated to a temperature of between about 30° C. to about 60° C., or from about 35° C. to about 55° C., or at about 45° C., 46° C., 47° C., 48° C., 49° C., 50° C., 51° C., 52° C., 53° C. or 54° C., e.g., at about 50° C.; (b) including in the mesoporous silica colloidal solution (i) a composition that does not comprise a primary amine group and, optionally, (ii) a PEG-silane compound to produce a nanoparticle containing amine groups (e.g., quatemarv, but also secondary and/or tertiary amine) which are not primary amine groups; and (c) hydrothermally treating the nanoparticles produced in step (b) by heating the nanoparticles at a temperature of between about 100° C. to about 150° C. (e.g., about between about 110° C. to about 140° C., such as between about 115° C. to about 135° C. or at about 120°, to yield the optionally PEGylated, monodisperse mesoporous silica nanoparticles (MSNPs); wherein the process can be one pot or in steps.

In the above process, (a) the alkoxysilane is 3-aminopropyltriethoxysilane (APTS), the solvent is N, N-dimethyl formamide (DMF) and the reporter is rhodamine B isothiocynate (RITC); and (b) the composition that does not comprise a primary amine group is N-trimethoxysilylpropyl-N,N,N-trimethyl ammonium chloride (50% in methanol. TMAC-silane) and the PEG-silane compound is methoxy(polyethyleneoxy)propyl]trimethoxysilane (Mw 550-750, 9-12 EO, PEG-silane).

Additional embodiments are directed to MSNPs and/or populations of MSNPs which are produced by the above methods.

In accordance with the present disclosure there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994. “Current Protocols in Molecular Biology” Volumes I-III: Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”: Hames & Higgins, eds., 1984, “Transcription And Translation”; Freshney, ed., 1986, “Animal Cell Culture”; IRL.

The invention is described further in the following non-limiting examples.

### Example 1

### Compositions for Targeted Integrin Alpha 7 Delivery

### Introduction

Obesity and the metabolic disease epidemic have led to an increase in morbidity and mortality. A rise in adipose thermogenic capacity via activation of brown or beige fat is a potential treatment for metabolic diseases. However, an understanding of how local factors control adipocyte fate is limited. Mice with a null mutation in the laminin α4 (LAMA4) gene (KO) exhibit resistance to obesity and enhanced expression of thermogenic fat markers in white adipose tissue (WAT). In this study, changes in WAT extracellular matrix composition in the absence of LAMA4 were evaluated using liquid chromatography/tandem mass spectrometry. KO-mice showed lower levels of collagen 1A1 and 3A1, and integrins α7 (ITA7) and β1 (ITB1). ITA7-ITB1 and collagen 1A1-3A1 protein levels were lower in brown adipose tissue compared to WAT in wild-type mice. Immunohistochemical staining confirmed lower levels and different spatial distribution of ITA7 in KO-WAT. In culture studies, ITA7 and LAMA4 levels decreased following a 12-day differentiation of adipose-derived stem cells into beige fat, and knock-down of ITA7 during differentiation increased beiging. These results demonstrate that extracellular matrix interactions regulate adipocyte thermogenic capacity and that ITA7 plays a role in beige adipose formation. A better understanding of the mechanisms underlying these interactions can be used to improve systemic energy metabolism and glucose homeostasis.

Obesity, a chronic imbalance in energy homeostasis between energy intake and energy expenditure, is a public health crisis and continues to be among the most important medical challenges in the U.S.A. Obesity creates a greater than $190 billion burden annually on American healthcare (Cawley & Meyerhoefer, 2012), with the percent of US medical expenditures devoted to treating obesity increasing 29% from 2001 to 20152. According to the most recent CDC statistics, over 70% of the United States population is overweight or obese, and more than 120 million people have diabetes or pre-diabetes. The development of therapeutic approaches that lessen or eliminate obesity-associated morbidity and mortality would have a transformative impact on the American population and healthcare spending. There are two types of adipose tissue in mammals: white adipose tissue (WAT) which is specialized for energy storage, and brown adipose tissue (BAT), specialized for energy expenditure and heat generation (Rosenwald et al., 2013; Saito et al., 2009; Zingaretti et al., 2009). The significant capacity of BAT for energy expenditure may be a mechanism for the treatment of metabolic disease, but the small volume of BAT in adults suggests that therapeutic potential is limited (Cannon & Nedergaard, 2010). However, “brown-like” adipocytes have been detected in human WAT. These “brown-in-white” or beige cells can be induced in WAT and could be a more viable approach (Spiegelman, 2013; Wu et al., 2013; Bi et al., 2014). Engineering BAT or beige adipose tissue depots in the lab for subsequent transplantation has also been explored as a treatment for metabolic disease in pre-clinical models (Vaicik et al., 2015; Tharp & Stahl, 2015; Blumenfeld et al., 2018). While investment has been made in designing therapeutics that target beige or brown adipose tissue, it remains difficult to maintain adipose tissue transformation in vivo. This results, in part, from incomplete knowledge concerning the mechanisms underlying beige and brown adipose tissue formation.

Recently, cell-extracellular matrix (ECM) interactions have been shown to influence beige and brown adipose formation and function (Vaicik et al., 2015; Alkhouli et al., 2013; Kuss et al., 2018: Tharp et al., 2018). In addition, it was found that the absence of laminin α4 protein (LAMA4) results in persistent beige adipose tissue formation, resistance to obesity, and enhanced metabolic function (Vaicik et al., 2018; Vaicik et al., 2014). Cells cultured on complex ECM exhibited increased expression of uncoupling protein 1 (UCP1), a primary marker of beige/brown adipocytes, when LAMA4 was absent (Cannon et al., 1982; Lin & Lin, 1980). While it is clear that the absence of LAMA4 results in increased expression of UCP1 in subcutaneous WAT (SubQ WAT) and BAT (Vaicik et al., 2018), the mechanism driving this behavior is unknown.

Disrupting the expression of a single protein can result in a broad range of changes to ECM properties (Mariman & Wang. 2010). The composition may be altered due to compensatory changes in other ECM proteins. The disruption can influence ECM assembly resulting in changes to the structure and mechanical properties. These broad changes indicate that the UCP1 expression observed in the absence of LAMA4 may result from other changes secondary to the absence of LAMA4. The fate of adipose tissue stem cells (ASC) is influenced by both the chemical composition and mechanical properties of the ECM, and cell-ECM interactions primarily occurs through integrins (Takagi, 2007). Integrins are a family of transmembrane receptors consisting of 18 α-subunits and 8 β-subunits (Hynes, 1992) that form at least 24 heterodimers. Integrins can activate signaling pathways that influence gene expression and cell function and may be a better target for therapeutic intervention than ECM molecules. Therefore, a fundamental understanding of matrix-integrin interactions that regulate the thermogenic capacity of adipose tissues will provide new insights into adipose function while informing new therapeutic options for engineering environments that stimulate ASCs beige differentiation.

Provided herein, the ECM composition of adipose tissue of LAMA4 knockout mice (KO) was examined in order to identify candidate ECM molecules and integrins potentially involved in regulating UCP1 expression. The data demonstrates that ECM molecules present at higher levels in WAT inhibit UCP1 expression, and that disrupting integrin interactions with the ECM can increase UCP1 expression. Aspects of the invention provide for integrin-matrix interactions as modulators of the thermogenic capacity of adipose tissue.

### Results

### Absence of Laminin α4 Alters ECM Composition and Integrin Levels.

Mice absent in laminin α4 (KO) are resistant to obesity and exhibit increased beiging of SubQ adipose tissue, increased energy expenditure, and enhanced insulin sensitivity (Vaicik et al., 2018). LC/MS-MS analysis was used to evaluate ECM composition of the SubQ adipose tissue from WT and KO mice. The total proteins in mice were evaluated and grouped according to presence in WT and KO mice and then categorized based on general biological processes or signaling pathways that the proteins belonged to. While there was significant overlap in proteins present in adipose tissue from the two WT and KO mice, specific differences were identified with regards to proteins involved in “binding” processes and “response to stimulus” signaling pathways.

Proteins in the “binding” pathway were examined and broad differences in the ECM profile between adipose from WT and KO mice were identified. In addition to the expected absence of LAMA4 (p<0.05), there was significantly lower levels of laminin gamma 1 (LAMC1) (p<0.01). Nearly all collagen molecules identified were lower in SubQ adipose tissue from KO mice, with both collagen 1A1 (CO1A1) and collagen 3A1 (CO3A1) showing significantly different levels (p<0.0001 and p<0.01 respectively). When evaluating proteins involved in cell binding and signaling, integrins α7 (ITA7) and β1 (ITB1) were found to be lower in the KO mice.

Expression of Beige Markers Varies with Extracellular Matrix Substrate.

Based on the results obtained from MS analysis, adipose derived stem cells (ASCs) isolated from WT SubQ WAT were cultured on surfaces coated with LAMA4, CO3A1 and CO1A1 and exposed to a beige differentiation protocol. Cells were able to proliferate and differentiate on all surfaces. However, UCP1 expression was lower with cells cultured on LAMA4, CO3A1 and CO1A1 surfaces relative to controls. Specifically, cells differentiated on a LAMA4 surface had a 51.4% reduction in UCP1, while cells differentiated on CO3A1 and CO1A1 exhibited a 91.16% and 71.7% reduction relative to uncoated surfaces, respectively. The expression of additional beige markers cytochrome c oxidase subunit isoform COX7A1 and peroxisome proliferator-activated receptor-γ coactivator-1α1 (PGC1A) was also analyzed. Similar to UCP1, there was a significant decrease in COX7A1 in differentiated cells on CO3A1, CO1A1 and LAMA4. PGC1A levels were also lower in cells on the coated surfaces, with a significant decrease on CO1A1 compared to uncoated surfaces. These results demonstrate that culture of ASCs on the ECM proteins present at higher levels in WT adipose tissue results in lower UCP1 expression. Adiponectin levels were analyzed as a general measure of adipogenic differentiation of the ASCs. Results showed lower expression levels of adiponectin on the ECM molecules, but the differences were not significant. The levels of reactive oxygen species (ROS) in culture were also examined in culture on the surfaces. UCP1 has been shown to reduce ROS production (Echtay & Brand, 2007). ROS levels were highest in undifferentiated cells that do not express UCP-1. ROS levels were lower in differentiated cells on LAMA4 and uncoated surfaces consistent with increased UCP1 expression. However, cells on LAMA4 coated surfaces had higher ROS levels compared to uncoated surface correlating with the lower UCP1 production on these surfaces.

Extracellular Matrix Proteins and Integrin Levels Vary with Adipose Depot.

The expression of UCP1 varies with adipose depot. BAT and two different kinds of WAT. SubQ and Epi, were isolated from WT mice, and the gene expression levels of ITA7, ITB1, CO1A1, LAMA4, and CO3A1 were examined by RT-PCR. As expected, UCP1 mRNA levels were significantly higher in BAT compared to both types of WAT. While UCP1 expression levels were higher in SubQ the difference with Epi was not significant. Adiponectin gene levels were lower in BAT compared to both Epi (p<0.0001), and SubQ WAT (0.01), with no difference among the WAT depots (p=0.09). Both ITA7 and ITB1 were significantly lower in BAT. ITA7 mRNA levels in BAT were 84% lower than Epi (p<0.0001) and 66% than SubQ (p=0.068). Interestingly. ITA7 levels were significantly higher in Epi when compared to SubQ (p=0.016). ITB1 levels were significantly lower in BAT compared to Epi (58% p<0.001), and lower but not statistically significant compared to SubQ (p=0.059).

The ECM proteins LAMA4, CO1A1 and CO3A1 were also lower in BAT. LAMA4 levels were significantly lower in comparison to Epi (p<0.0001) and lower than SubQ WAT (p=0.1). LAMA4 levels in Epi were significantly higher than SubQ WAT (p=0.018). Additionally, CO1A1 and CO3A1 levels were lower in BAT compared to SubQ (p<0.0001), and higher in SubQ compared to Epi (p=0.0076 in CO1A1 andp=0.0002 in CO3A1).

UCP1 protein levels were analyzed by western blot and results confirmed RT-PCR results, with higher UCP1 levels in BAT compared to other adipose depots but no difference between SubQ and Epi. Protein levels of ITA7 were also greater in both Epi and SubQ WAT compared to BAT. A broad observation is that UCP1 levels inversely correlate with LAMA4, ITA7, and ITB1 in adipose tissues. Integrin α7 protein expression in subcutaneous adipose tissue.

A confocal imaging and staining protocol was used to investigate the distribution of ITA7 positive cells in adipose tissue. Adipocytes, vasculature, and ITA7 were simultaneously stained in adipose tissue from KO and WT mice. The volume of ITA7 was 64% lower in SubQ adipose tissue of KO mice compared to WT (1338±296 μm³ in KO vs. 3713±409 m³ in WT, p<0.0001). Adipose precursor cells generally take on a perivascular phenotype (Shen et al., 2011). In examining both KO and WT adipose tissue, ITA7 staining was observed in cells present in the stromal tissue (ITA7⁺ cells) as well as lining the vasculature. Quantitative, the colocalization between ITA7 and vessels (lectin) was calculated. 26±5% of ITA7 was colocalized with lectin in KO mice and 17±2% of ITA7 was colocalized with lectin in WT. Although greater ITA7 and lectin colocalization was observed in KO tissue, the difference did not reach the criteria for significance (p<0.05).

To get a further analysis on the tissue distribution of ITA7⁺ cells, cytoNet was used, which is a robust method to quantify the spatial organization of cell communities. The analysis of spatial proximity of ITA7⁺ cells with both adipocytes and vessels was performed by evaluating the proximity of ITA7⁺ cells in relation to a threshold distance. Results showed that ITA7⁺ cells in KO tissue were on average closer to vessels than ITA7⁺ cells in WT tissue (p=0.046). The number of ITA7 positive cells was also quantified using cytoNet. There was a greater number of ITA7⁺ cells in WT tissue compared to KO, although this difference was not significant (p=0.059), consistent with both MS analysis and standard analysis of the stains.

Adipose tissues from KO and WT mice were stained for BODIPY (adipocytes), CD68 (a macrophage marker) and ITA7 in order to gain further insight into the types of cells expressing ITA7. Quantitatively, the amount of CD68 cells was significantly lower in SubQ adipose tissue of KO mice compared to WT (501±1305 μm³ in KO vs. 4034±697 μm³ in WT, p=0.0029) suggesting a decrease of macrophages in KO animals. Interestingly, the colocalization analysis with ITA7 showed that the majority of CD68 are also positive for ITA7 with no significant difference between KO and WT adipose tissue (83±5% of CD68 cells in KO vs. 72±4% in WT, p=0.07). From the overall ITA7 volume, 16±5% in KO and 26±4% in WT were CD68 cells. The majority of the ITA7 positive regions lined differentiated adipocytes. These results suggest that ITA7 is expressed in both macrophages and adipocytes.

In addition to the ITA7 analysis, other tissue characteristics were analyzed including adipocyte size and vascular density. Mean adipocyte diameter was significantly smaller in KO SubQ adipose compared to WT (50.6±1 μm in KO vs. 55.9±1.1 μm in WT, p=0.0004 F_{(1,547)}=12.7; n=550 adipocytes from 7 animals). Vascular parameters were not different between the two conditions. Vascular diameter was 4.7±0.14 μm in WT, and 5.0±0.15 μm in KO (p=0.12, F_{(1,348)}=2.3; n=350 vascular diameters from 7 animals). Vascular volume was 1164±291.5 μm²/area for KO and 1613±243.9 μm²/area for WT (F_{(1,49)}=0.018, p=0.89; n=50 images from 7 animals). These results were further confirmed by cytoNet analysis. The number of vessels was not different in WT and KO images. The average number of vessels per image was 4±0.5 in KO and 3.4±0.5 in WT (p=0.45, n=5 images per condition).

### Integrin α7 and Laminin α4 Decrease During Beige Differentiation.

Human adipose derived stem cells (hASCs) were next induced to form beige adipocytes and ITA7, LAMA4 and UCP1 levels were evaluated during the time course of differentiation. Phase microscopy and immunofluorescence confirm the morphological changes of the cells when differentiated and the lipid loading that occurs. UCP1 levels gradually increased over the 12 days of differentiation with the greatest increase from 10 to 12 days. ITA7 and LAMA4 levels showed a significant increase from day 0 to day 8 over the time course where there was not a significant increase in UCP1 expression differentiation. However, from day 8 to day 12 LAMA4 and ITA7 levels showed an inverse relationship with UCP1 with a significant decrease level of expression from day 10 to day 12 simultaneous with the dramatic increase in UCP1. At 8 days, a major increase in adiponectin also occurs, with no further significant changes over the course of differentiation. Similarly, other beige markers such as PGC1A, COX7A1, cell death activator CIDE-A (CIDEA) and iodothyronine deiodinase 2 (D102) showed a significant increase between d0 and d8 with no further increase between d8 and d12.

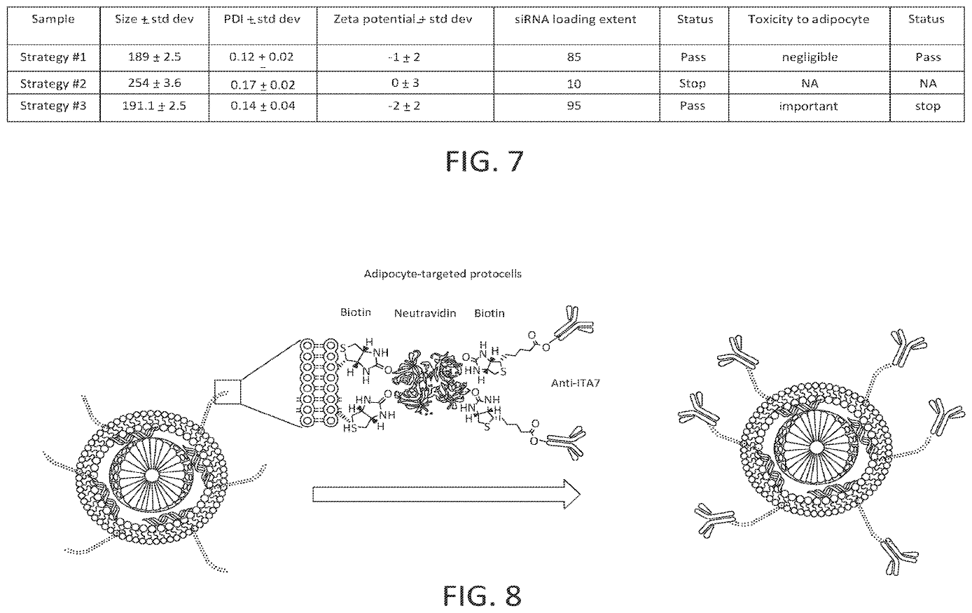

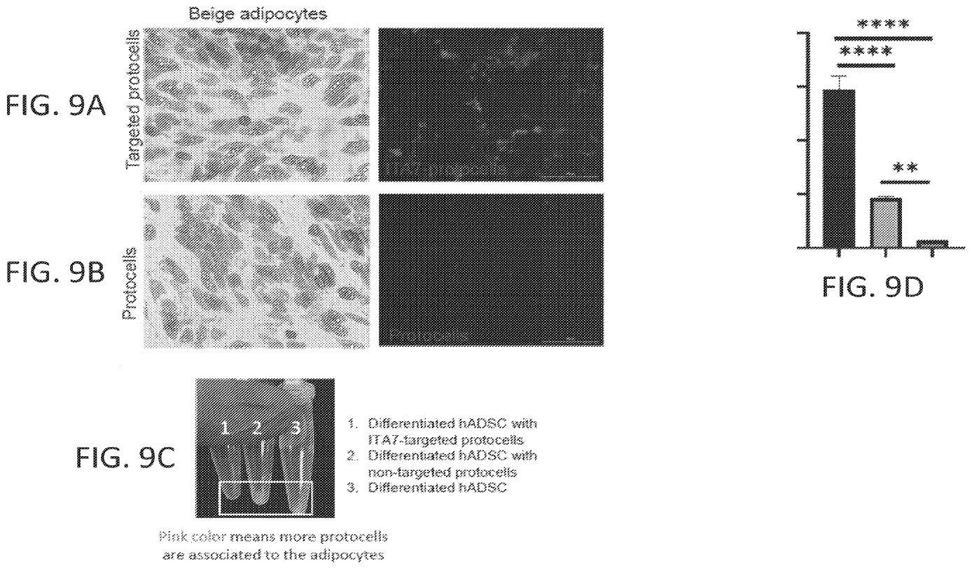

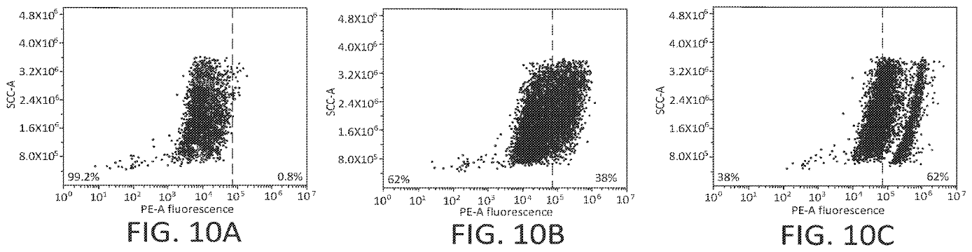

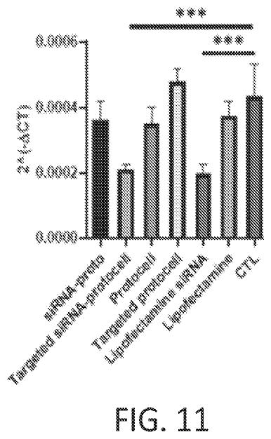

Knocking down integrin α7 leads to increased expression of UCP1. Based on these data integrin α7 serves as a target for therapeutic intervention. To evaluate ITA7 regulation as a means for increasing beige adipose formation, ASCs were transfected with ITA7 siRNA during adipogenic differentiation. In ASCs transfected with ITA7 siRNA, ITA7 gene levels were reduced by 59% (from 6.805±0.41 to 2.806±0.38. These ASCs also exhibited a substantial increase in UCP1 and COX7A1 expression. Interestingly, LAMA4 was also downregulated (FIG. 7D) in the cells transfected with ITA7 siRNA. Adiponectin and C/EBPalpha expression was not different between groups suggesting that basic adipogenesis of the cells was not affected. Indeed, brightfield images of differentiated adipocytes show lipid loading in cells with or without ITA7 knockdown.

### DISCUSSION

Provided herein extracellular matrix-cell interactions that play a role in beige adipocyte differentiation were identified. Laminin α4 KO mice are resistant to obesity, exhibit enhanced energy expenditure, and increased beige adipose formation (Vaicik et al., 2018). Broad changes in adipose ECM composition were observed in mice. The overall ECM composition is altered in complex ways, including a reduction in a number of collagen isoforms in addition to LAMA4. In addition, ITA7 was also lower in comparison to adipose from WT mice with levels appearing to correlate with LAMA4 level in multiple tests. The results of these studies provide a new list of targets for treatment of obesity or metabolic disease.

The basement membrane of adipocytes consists primarily of various laminin isoforms and collagen (Kalluri, 2003). These components provide attachment points for integrins and other extracellular matrix receptors, such as CD36 and CD44; anchored in the adipocyte membrane (Bonnans et al., 2014). The importance of the matrix interaction with adipogenesis has been previously demonstrated. For instance, blocking collagen synthesis in ASCs inhibits differentiation; and collagen VI is sufficient to restore the adipogenic potential (Mariman & Wang. 2010). Provided herein is insight into the differences between ECM in beige and white adipose tissue by looking into ECM protein changes in the SubQ adipose tissue from WT and KO mice. LC/MS analysis revealed changes in the ECM profile. CO1A1 and CO3A1 were at significantly lower levels in KO mice compared to WT. Additionally, CO3A1 levels were lower in BAT compared to WAT suggesting that thermogenic adipose tissue (brown and beige) have lower levels of CO3A1 and CO1A1. Collagen deposition makes the ECM less flexible which is something that occurs during fibrosis, the hallmark of obesity and type 2 diabetes (Lin et al., 2016; Sun et al., 2013). CO1A1 and CO3A1 are indeed two markers of fibrosis, and it has been shown that they play a role in fibro-adipogenic precursor differentiation and fat deposition (Cordani et al., 2014). Higher levels of CO1A1 and CO3A1 were associated with higher levels of fibro-adipogenic progenitors in muscle as well as greater adipose tissue deposition. The decrease in ECM proteins that was observed in the results, specifically in CO1A1 and CO3A1 can alter the flexibility of the tissue suggesting that a tissue more flexible favors the development of thermogenic adipose tissue.

ASC plated on CO1A1, CO3A1 or LAMA4 surfaces and induced to differentiate into beige adipocytes, exhibited lower levels of UCP1 compared to cells differentiated on uncoated tissue culture plates. These differences suggest that a coordinated change in ECM proteins may occur during the beiging of WAT and provides multiple potential targets when developing therapeutic approaches. The ECM composition in adipose tissue changes during development and is different between adipose depots (Mariman & Wang, 2010; Kubo et al., 2000; Mori et al., 2014). For instance, SubQ WAT of rats express higher levels of type I and III collagens compared to visceral WAT (Mori et al., 2014). The results showed that CO3A1 is indeed greater in SubQ adipose tissue compared to visceral adipose tissue and BAT. Lowering levels of CO3A1 in SubQ adipose tissue may create an environment that helps promoting differentiation of stem cells into beige adipocytes. ECM proteins are often linked to the cytoskeleton through integrins which are a class of transmembrane proteins. Integrins are heterodimers of alpha and beta subunits, the combination of each dictates ligand specificity (Barczyk et al., 2010). Integrin interactions with ECM molecules can activate signaling pathways that influence gene expression and cell function. Classification analysis of the protein results suggest that cell binding and integrin signaling are reduced in the KO beige adipose tissue. Specifically, ITA7 and ITB1 were reduced in the KO beige adipose tissues. To confirm these results the levels of ITA7 and ITB1 in WAT and BAT of WT mice were evaluated. The levels of both integrins were lower in BAT compared to WAT. These results indicate that low levels of these integrins are characteristic of the ECM of thermogenic adipose tissue. Therefore, lowering levels of these integrins in WAT can induce beiging of the adipose tissue. ITB1 plays a role in mediating cellular interactions with many ECM proteins (Pope et al., 2016). Also, ITB1 is upregulated during hypertrophic growth, as is the activity of downstream effector kinases (Villa-Diaz et al., 2016). ITB1 is widely present in many cells, playing a role in a broad range of cellular processes, making ITB1 a difficult target for therapeutic purposes. ITA7 has been linked with the regulation of cell adhesion and migration (Mielenz et al., 2001), and although its role in adipocytes has not been studied, ITA7 is strongly upregulated in differentiated white adipocytes compared to ASC (Malekpour-Dehkordi et al., 2019; Morandi et al., 2016). The results showed that ITA7 levels in hASCs are also upregulated in the initial stages of beige adipocyte differentiation, but at later stages it decreases as UCP1 levels increase. These results suggest that ITA7 upregulation is needed to start the differentiation process and might induce changes in the shape of adipocytes and facilitate their localization at laminin-rich sites as it does in other cells such as myoblasts (Schober et al., 2009; Tran et al., 2007; Ziober et al., 1997). At later stages, the results demonstrate that differentiated beige adipocytes present lower levels of ITA7. In contrast, further upregulation of ITA7 have been seen in insulin resistant and hypertrophied 3T3-11 adipocytes (Malekpour-Dehkordi et al., 2019).

Recent studies have shown that brown/beige adipocytes can also be derived from muscle progenitor cells. Muscle progenitor cells can be differentiated, in part, by their ITA7 expression. Progenitor cells negative for ITA7 have a significantly higher propensity for UCP1 expression consistent with results we have seen here (Gorski et al., 2018: Joe et al., 2010; Schulz et al., 2011; Uezumi et al., 2010). To confirm the implication of ITA7 in modulating the metabolic function of adipocytes, the expression of ITA7 was knocked down in ASC and induce them to beige differentiation. In agreement with the other results, ITA7 knockdown is able to enhance UCP1 expression making it a possible target to induce beige adipose tissue.

Staining for ITA7 in SubQ adipose tissue, not only confirms that ITA7 is lower in KO mice but also provides insight into the potential function of these cells. Cells positive for ITA7 were present both in the stroma and perivascularly, possibly suggesting that ITA7 stains multiple cell types, precursor cells in the perivascular space and other cell types in the stroma. Indeed, ITA7 stained the majority of CD68 cells in the stroma indicating that a subset of the ITA7 positive cells are macrophages. Macrophages have been shown to play a role in adipogenesis of fibroadipogenic progenitors (Moratal et al., 2018), but it remains unknown the role that immune cells have on beige adipocytes. CD68 volume was lower in KO tissue, in agreement with previous studies showing that CD68 tend to increase with BMI and to decrease with agonists for peroxisome proliferator-activated receptor- (PPAR-) (Di Gregorio et al., 2005). ITA7 pre-sent in the adipose tissue from KO mice exhibited a different distribution than in tissue from WT animals. It is important to understand the cell spatial distribution since it has been demonstrated that there is a close spatial and temporal interrelationship between blood vessel formation, adipogenesis and stromal cells (Nishimura et al., 2007). In KO adipose the ITA7⁺ cells in the stroma were closer to the vessels on average. Although it the reason for this difference in ITA7 spatial distribution is unclear, it may result, in part, from the difference in size of adipocytes in WT and KO adipose tissue. The increase in beige adipose tissue means that the adipocytes are smaller in KO mice which can reduce the effective distance from vessels. The role of the ITA7 spatial distribution in beige adipogenesis remains to be studied. More ITA7 positive cells could indicate an increase in ASC differentiation to white adipocytes, since ITA7 increases during the process of white adipocyte differentiation (Malekpour-Dehkordi et al., 2019; Morandi et al., 2016). Less hyperplasia (increased in adipocyte number) in SubQ of KO mice could explain their resistant to obesity and enhanced metabolic function that was previously observed (Vaicik et al., 2018). Therefore, lowering ITA7 in tissue can switch the differentiation of stromal cells from white to beige adipocytes.

All of the results indicate a direct correlation between LAMA4 expression and ITA7. ITA7 specifically binds to laminin, the substrate that is an important ECM component of adipocytes containing large fat vacuoles (Frith et al., 2012; Liu et al., 2005). A study by Morandi et al. (2016) performed gene profiling of 18 alpha integrins in ASCs and differentiated adipocytes and suggested that ITA7 is the responsible for laminin-dependent signaling in differentiating ASCs. In this study it was demonstrated that ITA7 levels regulates beige adipocyte differentiation and downregulation of ITA7 occurs in LAMA4 KO adipose tissue. Therefore, ITA7 could be responsible for LAMA4-dependent signaling in beige differentiating ASCs. Future studies will look into the Notch signaling pathway as a mechanism of LAMA4 regulation of UCP1 expression. In the absence of LAMA4 the Notch pathway is suppressed (Sun et al., 2019) and inhibition of Notch signaling has been shown to result in browning of white adipocytes (Thyboll et al., 2002).

### Conclusion

The results of this study provide unique insights into adipose function by investigating changes in extracellular matrix components that alter thermogenic capacity of adipocytes. Specifically, it was found that ITA7 modulates the metabolic function of the adipocytes. In addition, the ECM of thermogenic adipose tissue is characterized to have lower levels of COTA1 and CO3A1 compared to WAT. This information identifies therapeutic targets for obesity and metabolic diseases. A better understanding of the underlying causes of these characteristics of brown and beige fat allows for specific manipulation of these cells to improve systemic energy metabolism and glucose homeostasis.

### Methods

Animal models. The development and characterization of laminin α4 knockout mice (KO) has been described previously (Vaicik et al., 2015). Wild type (WT) mice used are C57 BL/6 mice (Charles River) as the KO mice were generated on this background. For the studies described here the mice were fed a standard chow diet ad libitum. All animal procedures were approved by the IACUC at the University of Chicago.
Tissue harvest. Mice at 13-15 weeks of age were sacrificed, and various adipose tissue depots harvested from both WT and KO mice. WAT was isolated from subcutaneous (SubQ) and epididymal (Epi) depots, and BAT was isolated from the interscapular region. The number of mice used per experiment are provided in each section below.
Mass spectrometry. Liquid chromatography/tandem mass spectrometry (LC/MS) was used to identify the ECM components and the relative quantities in WAT from WT and KO mice. SubQ adipose tissue was frozen in liquid nitrogen. After mincing, the tissue was suspended in CHAPs buffer in PBS and stirred in a hot plate until dissolved, followed by incubation overnight at 37° C. with 135 RPM. The buffer was removed and tissue sus-pended in Tris/EDTA buffer prior to freezing at −80° C. To remove lipids, the tissue was thawed and incubated in isopropanol for 48 hours. The adipose tissue samples were solubilized in Rapigest (Waters Milford, MA) containing 50 mM ammonium bicarbonate, and sonicated at 2×15 s with 0.5 s pulsing. DTT and IAN were added to reduce and alkylate, respectively, the proteins prior to Lys-C/Trypsin digestion for 45 minutes and then overnight. Samples were then acidified with TFA to remove Rapigest, and protein/peptide concentration was determined via amino acid analysis prior to injecting samples into the mass spectrometer. LC/MS-MS analysis was performed on a Thermo Scientific Orbitrap Elite mass spectrometer coupled to a Waters nanoACQUITY UPLC system, using a Waters Symmetry® C18 180 μm 9 20 mm trap column and a 1.7 lm, 75 μm×250 mm nanoAcquitv™ UPLC™ column (38° C.) for peptide separation across a 140-minute run. MS was acquired in the Orbitrap (300-2000 m/z) using 1 microscan, a full max ion time of 500 ms, and a resolution of 30 000. MS-MS was acquired in the Ion Trap using collision-induced dissociation (CID) for up to 15 MS-MS analyses per MS scan. Minimum signal required was 500, dynamic exclusion was set to 60 seconds, and the normalized collision energy was 35. Mascot Distiller was used to generate peak lists, and the Mascot search algorithm was used for searching against the Swiss Protein database with and without taxonomy restricted to human. Carbamidomethyl Cys, citrullination of Arg, oxida-tion of Met, Pro, and Tyr, and Nitrosylation of Cys were entered as variable modifications. Two missed tryptic cleavages were allowed, precursor mass tolerance was set to 10 ppm, and fragment mass tolerance was set to 0.2 Da. The significance threshold was set at p<0.05, with a False Discovery Rate (FDR) of 5%. Three biological samples were run, with 3 technical replicates for each sample. Proteins were quantified using the exponentially modified protein abundance index (emPAI), which relates the number of unique peptides observed for a specific protein to the number of observable peptides in the sample (Ishihama et al., 2005).

TABLE 1
Quantitative real-time polymerase chain reaction primer sequences.

| Primer | Forward primer | Reverse primer |
|---|---|---|
| 18 s (m) | 5′- | 5′- |
| | GTAACCCGTTGAACCCCAT | CCATCCAATCGGTAGTAGC |
| | T (SEQ ID NO: 1) | G (SEQ ID NO: 2) |
| UCP1 | 5′- | 5′- |
| (m) | ACTGCCACACCTCCAGTCA | CTTTGCCTCACTCAGGATT |
| | TT (SEQ ID NO: 3) | GG (SEQ ID NO: 4) |
| ITA7 | 5′- | 5′- |
| (m) | GATCGTCCGAGCCAACAT | CTAACAGCCCAGCCAGCAC |
| | CACA (SEQ ID NO: 5) | T (SEQ ID NO: 6) |
| Adiponectin | GAATCATTATGACGGCAG | TCATGTACACCGTGATGTG |
| (m) | CA (SEQ ID NO: 7) | GTA (SEQ ID NO: 8) |
| COLIA1 | CGTATCACCAAACTCAGA | GAAGCAAAGTTTCCTCCAA |
| (m) | AG (SEQ ID NO: 9) | G (SEQ ID NO: 10) |
| COL3A1 | 5′- | 5′- |
| (m) | CTGGAGAACCTGGTGCAA | CCTCGGAAGCCACTAGGAC |
| | AT (SEQ ID NO: 11) | (SEQ ID NO: 12) |
| ITB1 | AATGTTTCAGTGCAGAGCC | TTGGGATGATGTCGGGAC |
| (m) | (SEQ ID NO: 13) | (SEQ ID NO: 14) |
| LAMA4 | 5′- | 5′- |
| (m) | GGAATACCTGAACGTGCA | GTGCCATCTGCCATCACAG |
| | CATGAGA (SEQ ID NO: 15) | AGATTCT (SEQ ID NO: 16) |
| GAPDH | 5′- | 5′- |
| (h) | TGACAACTTTGGTATYCGT | AGGCAGGGATGATGTTCTG |
| | GGAAGG (SEQ ID NO: 17) | GAGAG (SEQ ID NO: 18) |
| UCP1 | 5′- | GAATACTCCCACTCCTCCA |
| (h) | TCTACGACACGGTCCAGG | GTC (SEQ ID NO: 20) |
| | AG (SEQ ID NO: 19) | |
| LAMA4 | 5′-GCGGCCGAGAAATGCA | 5′- |
| (h) | (SEQ ID NO: 21) | AGTCGCAGGGCACACATTC |
| | | (SEQ ID NO: 22) |
| ITA7 (h) | 5′- | 5- |
| | ACCAATACCCTGACCTGCT | CTATAGCTGCTGGGGACTG |
| | G (SEQ ID NO: 23) | C (SEQ ID NO: 24) |
| Adiponectin | 5′- | 5′- |
| (h) | AAGGAGATCCAGGTCTTA | ACCTTCAGCCCCGGGTAC |
| | TTGG (SEQ ID NO: 25) | (SEQ ID NO: 26) |

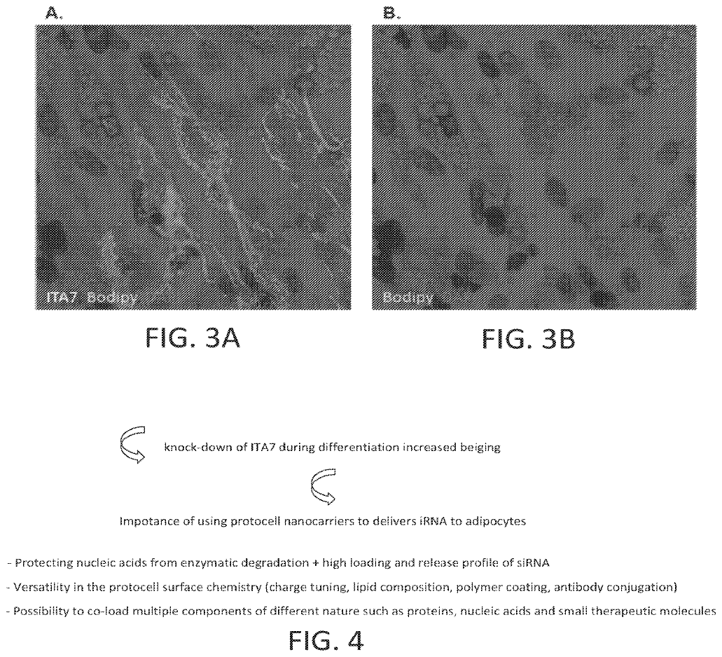

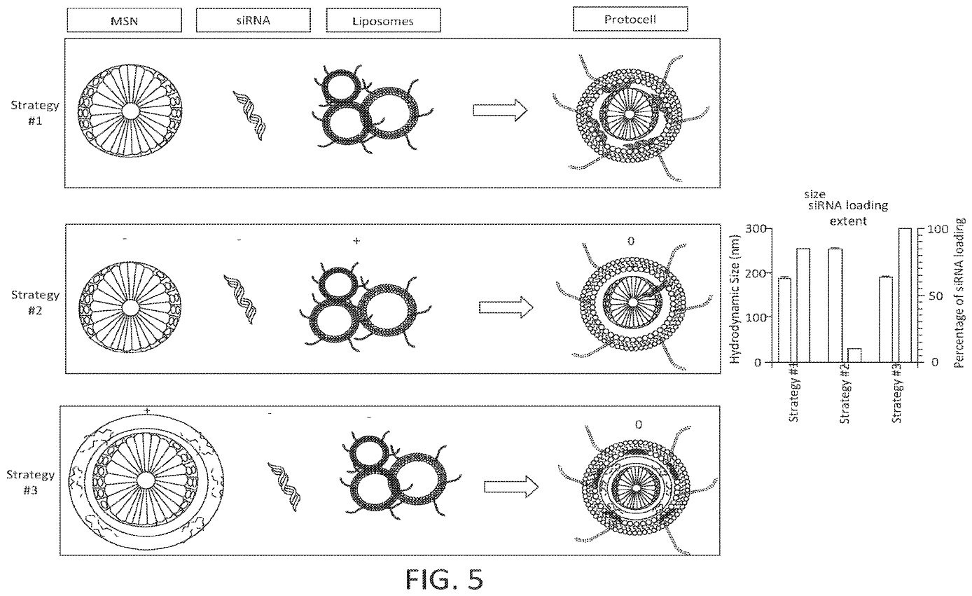

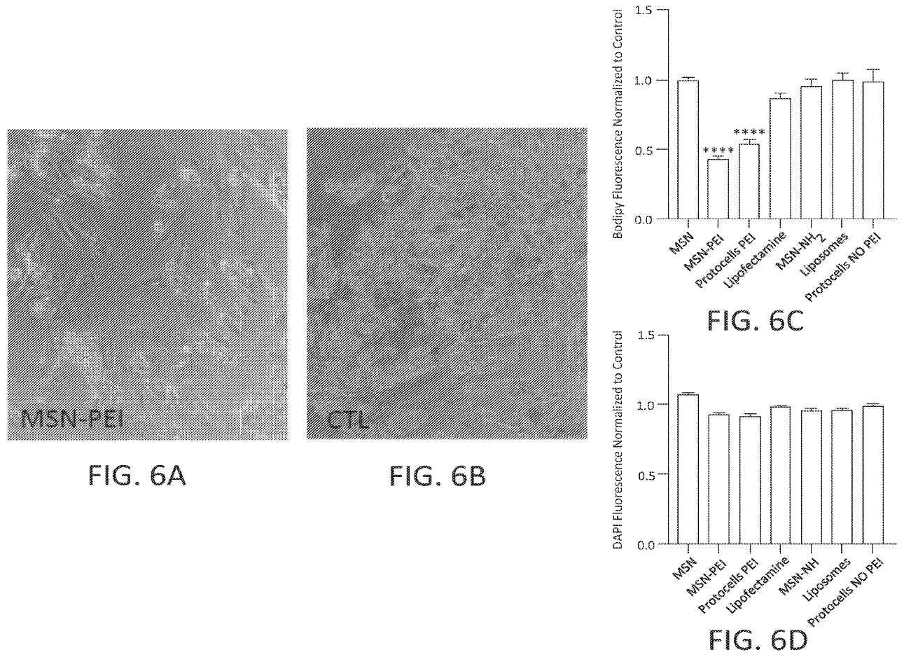

Immunohistochemistry. SubQ fat was harvested from animals using sterile techniques, cut into small pieces and fixed in 4% paraformaldehyde for 24 hours at 4 degrees. Tissue pieces were then washed with phosphate buffered saline (PBS), permeabilized with 0.5% triton in PBS and blocked with 10% donkey serum; followed by incubation with either 1) ITA7 antibody (1:100, Novus Biologics NBP1-86,118) and Griffonia simplicifolia isolectin conjugated with Rhodamine to labels endothelial cells, or 2) ITA7 antibody and CD68 antibody (1:100, Santa Cruz sc-70761) to label macrophages for 48 hours at 4 degrees. After washes (3×15 minutes), the tissues were incubated with second antibodies (Alexa Fluor 647 Donkey Anti-Rabbit IgG and/or TRITC Goat Anti-Mouse, IgG), BODIPY to stain lipid droplets and DAPI to stain the nuclei, for 2 hours at room temperature.
Confocal imaging. A confocal laser-scanning microscope was used to image samples (Leica TCS SP8 Confocal Microscope; Buffalo Grove, IL). Adipocytes labeled by BODIPY and ITA7 labeled by Alexa Fluor 647 were imaged simultaneously in two channels, with Rhodamine-labeled endothelial tissue or TRITC CD68-labeled macrophages, and DAPI nuclei imaged in series. Confocal image stacks were obtained with a 63×oil immersion lens (NA 1.4). Laser intensity, confocal aperture, and photomultiplier gain were kept constant across samples.
Image processing and analysis: Confocal image stacks were processed and analyzed using Leica LASX software version 3.5.2 (Wetzlar, Germany). The Alexa 647 and Rhodamine channels were thresholded and the intersection between both layers was quantified in order to characterize the colocalization between ITA7 and endothelial cells. In separate tissue, the colocalization between ITA7 and CD68 positive cells was also quantified. Using 3D images, the Alexa 647, Rhodamine or TRITC and intersection area per cell were calculated.
cytoNet image analysis: cytoNet is a software accessible over the web that quantifies the spatial relationships in cell communities using principles of graph theory and evaluates the effect of cell-cell interactions on individual cell phenotypes (https://www.braininitiative.org/toolmakers/resources/cytonet/). Image files were segmented manually and provided as input to cytoNet. Because blood vessels have noncircular shapes, the minimum distance between object perimeters was computed in order to define graph edges. The resulting cell-to-cell perimeter distance and cells characteristics were analyzed in WT and KO SubQ WAT images.
RNA isolation and quantitative RT-PCR. RNA from tissue and cells was isolated and purified using a Qiagen RNeasy Mini Kit (Valencia, CA) according to manufacturer guidelines. mRNA concentrations were measured using a Take3 Micro-Volume Plate (BioTek. Winooski, VT), then normalized to 150 ng of mRNA for conversion to cDNA. cDNA was synthetized using random hexamer primers and an iScript cDNA-synthesis kit (Biorad). The quantitative RT-PCR reactions were performed using the So Advanced™ Universal SYBER Green Supermix kit (Biorad) in a CFX96 Touch Real-Time PCR Detection System (BioRad, Hercules. CA). Fold expression levels were calculated using the 2^{ΔΔCt} method. Transcript levels were normalized to 18S ribosomal RNA levels in all experiments that used mice cells (FIGS. 3, 4, 7), or the GAPDH reference gene in experiments with human cells (FIG. 6). Primer (Table 1) specificity was tested by the assessing the melting curve.
Western blot analysis. Cells were lysed using CelLytic (Sigma, St. Louis, MO) and tissue was lysed using RIPA buffer (EMD Millipore, Billerica, MA) and protease inhibitor cocktail (Fischer Scientific, Waltham, MA). Protein concentration was determined using the Bio-Rad DC protein assay (Bio-Rad Laboratories, Hercules. CA). Samples were diluted in water and loading buffer (LI-COR, Lincoln, NE), electrophoretically separated under denaturing conditions on 4-15% SDS-PAGE Criterion gels (Bio-Rad) and transferred to nitrocellulose membranes (Bio-Rad). Membranes were blocked in Intercept Protein-Free Blocking Buffer (LI-COR), followed by an overnight incubation with primary antibody for UCP1 (Invitrogen, Carlsbad, CA; rabbit polyclonal, #PA1-24894), ITA7 (Novus, Centennial CO; rabbit polyclonal, #NBP1-74207) or GAPDH (Santa Cruz; mouse monoclonal, #sc-32233) diluted in InterceptK Protein-Free Antibody Diluent (LI-COR). Membranes were incubated with appropriate IRDve secondary antibodies (LI-COR), and immunodetection was performed using near-infrared Odyssey Fc Imaging System (LI-COR). Images were obtained with Image Studio Software (LI-COR).
Cell culture primary adipose-derived stem cells. Isolation: SubQ adipose depots were harvested from euthanized KO and WT mice and digested in collagenase type I. The adipose-derived stem cells were enzymatically isolated following a previously published protocol (Yu et al., 2011). Briefly, digestion was performed in an orbital shaker at 37° C. for 60 minutes. The digest was then centrifuged, and the floating adipocyte layer discarded. The pelleted stromal vascular fraction was washed two times with complete media (CM, Dulbecco's modified Eagle's Ham's F12 medium supplemented with 10% fetal bovine serum, and 1% antibiotic-antimycotic) and plated on tissue culture plastic. Cells at passages 2 to 5 were used for experiments.
Adipose derived stem cells beige differentiation: Cells (passage 2-4) were cultured in 24 well tissue culture plates and incubated in CM. When cells reached 95% confluence differentiation was initiated. For beige differentiation cells were incubated for 4 days in induction media (CM with 5 μg/ml insulin, 10 μM forskolin, 2 μg/ml dexamethasone, 125 μM indomethacin, 0.5 μM Rosiglitazone and 1 nM triiodothvronine) and then maintained in CM with 5 μg/ml insulin, 10 μM forskolin, 1 μM rosiglitazone and 1 nM triiodothyronine. Differentiation media was changed every other day until harvest.
siRNA transfection: Adipocytes were transfected at day 10 of differentiation. The lipofectamine RNAiMAX preparation was carried out following the manufacturer's instructions. For a 24 well plate, the lipofectamine RNAiMAX reagent (3 μl per well) and the siRNA (10 μM) were diluted separately in serum free media and mixed by pipetting. The siRNA-RNAiMAX mix was left to incubate for 5 minutes at room temperature after which the siRNA-RNAiMAX mix was added on top of the adherent cells. Adipocytes were transfected twice during the differentiation process and harvested 3 days after the last transfection (14 days after differentiation).
Surface coated plates: Collagen 3A1, Collagen 1A1 and Laminin α4 recombinant proteins (R&D Systems) were reconstituted in PBS to obtain a working concentration of 20 μg/ml. 24 wells-tissue culture plates were coated with 300 μl of each reconstituted protein and incubated for 4 hours at 37° C. Excess fluid was removed, and the plates allowed to dry before introducing cells and media. All protocols were performed under sterile conditions.
Hydrogen peroxide assay. The ROS-Glo H₂O₂ Assay uses a modified luciferin substrate, based on boronate oxidation, which reacts directly with hydrogen peroxide (H₂O₂) to generate a luciferin precursor. Upon addition of detection reagent, precursor is converted to luciferin and Ultra-Glo Recombinant Luciferase included in the detection reagent produces a light signal proportional to the level of H₂O₂ in the sample. Briefly, ASCs were differentiated in LAMA4 coated surfaces or uncoated surfaces in 48 wells. Negative control cells were not differentiated and kept in growth media. 6 hours before the end of the differentiation 150 μl of a mixture of H₂O₂ substrate and H₂O₂ dilution buffer was added to each well. Six hours later, 50 μl of the media was mixed with 50 μl of ROS-GLO Detection Solution in a separate plate and incubated at room temperature for 20 minutes. Luminescence was read by Take3 Micro-Volume Plate (BioTek, Winooski. VT).
Cell culture and differentiation of human adipose derived stem cells. Commercially available hASCs (PT-5006, Batch 0,000,535,975, Lonza) were used at passage 2 or 3. Cells were seeded at a density of 25,000 cells/cm² in CM. The media was changed every other day. When cells reached confluence differentiation was initiated (designated as day 0). Cells were then maintained in Dulbecco's modified Eagle's Ham's F12 (DMEM/F12) media supplemented with 850 nM insulin, 250 μM isobutyl-methylxanthine, 125 nM indomethacin, 0.5 μM dexamethasone, 1 μM rosiglitazone and 120 nM triiodothyronine. Differentiation media was changed every other day.
Statistical methods. Graphpad Prism Software 6 (GraphPad Software, Inc., La Jolla, CA) was used to run unpaired T tests with Welch's correction and one way analysis of variance (ANOVA) tests with Tukey or Dunnet multiple comparison analysis (as specified in each figure) to determine differences between groups. Statistical significance was defined when p<0.05. All results are presented as mean±standard error of the mean (SEM).

### Example 2

### Exemplary Components for Lipid Layer

In one embodiment, the lipid comprises one or more zwitterionic lipids such as DPPC (1,2-dipalmitoyl-sn-glycero-3-phosphocholine), DOPE, DOPC, DSPC, DMPC, DPPE, cholesterol, DSPE PEG (1,2-distearoyl-sn-glycero-3-phosphoethanolamine-N-[(polyethylene glycol)-2000]), DSPE PEG-Biotin (1,2-distearoyl-sn-glycero-3-phosphoethanolamine-N-[biotinyl(polyethylene glycol)-2000](ammonium salt), or another PEGylated zwitterionic lipid, or any combination thereof.

In one embodiment, the lipid comprises one or more positively charged lipids. e.g., DODMA (1,2-dioleyloxy-3-dimethylaminopropane) or DOTAP (1,2-dioleoyl-3-trimethylammonium-propane (chloride salt)).

In one embodiment, the lipid comprises one or more negatively charged lipids. e.g., DMPG (1,2-dimyristoyl-sn-glycero-3-phospho-(1′-rac-glycerol) (sodium salt), glycerol based DPPG or DSPG or serine-based DMPS, DPPS, or DSPS.

In one embodiment, the lipid comprises one or more pegylated lipids (e.g., DSPE-PEG-azide, DSPE-PEG-amine, DSPE-PEG-COOH, or others).

In one embodiment, the protocell comprises a ligand that binds adipocytes. e.g., antibodies that bind ITGA7, e.g., a monoclonal or polyclonal antibody such as rabbit anti-human polyclonal antibodies or antibodies that bind laminin alpha 4, e.g., polyclonal anti-human laminin alpha 4 antibodies.

The cargo delivered by the protocells may include, for example, siRNA specific for integrin α7/ITGA7 or laminin alpha 4 or may be forskolin. Examples of siRNA ITGA/7 sequences include:

(SEQ ID NO: 27)
Gcaucaagagcuucggguatt
reverse
(SEQ ID NO: 28)
uagcccgaagcucuugaugctt
(SEQ ID NO: 29)
gcugcccacucuacaguutt
reverse
(SEQ ID NO: 30)
aagcuguagaguggucagctt

and those from Santa Cruz Biotech Catalog number: sc-60018.

Exemplary ITGA7 sequences include:

| (SEQ ID NO: 31) | |
|---|---|
| MAPFATPMVQALTTTRIQRQAEGFQCWRECGTRRSPFEGKETCA | |
| HRYEARQRVDQILETRDMIGRCFVLSQDLAIRDELDGGEWKFCEGRPQGHEQFGFCQQ | |
| GTAAAFSPDSHYLLFGAPGTYNWKGTARVELCAQGSADLAHLDDGPYEAGGEKEQDPR | |
| LIPVPANSYFGFSIDSGKGLVRAEELSFVAGAPRANHKGAVVILRKDSASRLVPEVML | |
| SGERLTSGFGYSLAVADLNSDGWPDLIVGAPYFFERQEELGGAVYVYLNQGGHWAGIS | |
| PLRLCGSPDSMFGISLAVLGDLNQDGFPDIAVGAPFDGDGKVFIYHGSSLGVVAKPSQ | |
| VLEGEAVGIKSFGYSLSGSLDMDGNQYPDLLVGSLADTAVLFRARPILHVSHEVSIAP | |
| RSIDLEQPNCAGGHSVCVDLRVCFSYIAVPSSYSPTVALDYVLDADTDRRLRGQVPRV | |
| TFLSRNLEEPKHQASGTVWLKHQHDRVCGDAMFQLQENVKDKLRAIVVTLSYSLQTPR | |
| LRRQAPGQGLPPVAPILNAHQPSTQRAEIHFLKOGCGEDKICQSNLQLVRARFCTRVS | |
| DTEFQPLPMDVDGTTALFALSGQPVIGLELMVTNLPSDPAQPQADGDDAHEAQLLVML | |
| PDSLHYSGVRALDPAEKPLCLSNENASHVECELGNPMKRGAQVTFYLILSTSGISIET | |
| TELEVELLLATISEQELHPVSARARVFIELPLSIAGMAIPQQLFFSGVVRGERAMQSE | |
| RDVGSKVKYEVTVSNQGQSLRTLGSAFLNIMWPHEIANGKWLLYPMQVELEGGQGPGQ | |
| KGLCSPRPNILHLDVDSRDRRRRELEPPEQQEPGERQEPSMSWWPVSSAEKKKNITLD | |
| CARGTANCVVFSCPLYSEDRAAVLHVWGRLWNSTFLEEYSAVKSLEVIVRANITVKSS | |
| IKNLMLRDASTVIPVMVYLDPMAVVAEGVPWWVILLAVLAGLLVLALLVLLLWKMGFF | |
| KRAKHPEATVPQYHAVKIPREDRQQFKEEKTGTILRNNWGSPRREGPDAHPILAADGH | |
| PELGPDGHPGPGTA | |
| or | |
| (SEQ ID NO: 32) | |
| MAGARSRDPWGASGICYLFGSLLVELLFSRAVAFNLDVMGALRK | |
| EGEPGSLFGFSVALHRQLQPRPQSWLLVGAPQALALPGQQANRTGGLFACPLSLEETD | |
| CYRVDIDQGADMQKESKENQWLGVSVRSQGPGGKIVTCAHRYEARQRVDQILETRDMI | |
| GRCFVLSQDLAIRDELDGGEWKFCEGRPQGHEQFGFCQQGTAAAFSPDSHYLLEGAPG | |
| TYNWKGLLFVTNIDSSDPDQLVYKTLDPADRLPGPAGDLALNSYLGESIDSGKGLVRA | |
| EELSFVAGAPRANHKGAVVILRKDSASRLVPEVMLSGERLTSGFGYSLAVADLNSDGW | |
| PDLIVGAPYFFERQEELGGAVYVYLNQGGHWAGISPLRLCGSPDSMFGISLAVLGDLN | |
| QDGFPDIAVGAPFDGDGKVFIYHGSSLGVVAKPSQVLEGEAVGIKSFGYSLSGSLDMD | |
| GNQYPDLLVGSLADTAVLFRARPILHVSHEVSIAPRSIDLEQPNCAGGHSVCVDLRVC | |
| FSYIAVPSSYSPTVALDYVLDADTDRRLRGQVPRVTELSRNLEEPKHQASGTVWLKHQ | |
| HDRVCGDAMFQLQENVKDKLRAIVVTLSYSLQTPRLRRQAPGOGLPPVAPILNAHQPS | |
| TQRAEIHFLKQGCGEDKICQSNLQLVRARFCTRVSDTEFQPLPMDVDGTTALFALSGQ | |
| PVIGLELMVTNLPSDPAQPQADGDDAHEAQLLVMLPDSLHYSGVRALDPAEKPLCLSN | |
| ENASHVECELGNPMKRGAQVTFYLILSTSGISIETTELEVELLLATISEQELHPVSAR | |
| ARVFIELPLSIAGMAIPQQLFFSGVVRGERAMQSERDVGSKVKYEVTVSNQGQSLRTL | |
| GSAFLNIMWPHEIANGKWLLYPMQVELEGGQGPGQKGLCSPRPNILHLDVDSRDRRRR | |
| ELEPPEQQEPGERQEPSMSWWPVSSAEKKKNITLDCARGTANCVVFSCPLYSFDRAAV | |
| LHVWGRLWNSTFLEEYSAVKSLEVIVRANITVKSSIKNLMLRDASTVIPVMVYLDPMA | |
| VVAEGVPWWVILLAVLAGLLVLALLVLLLWKMGFFKRAKHPEATVPQYHAVKIPREDR | |
| QQFKEEKTGTILRNNWGSPRREGPDAHPILAADGHPELGPDGHPGPGTA | |
| or | |
| (SEQ ID NO: 33) | |
| MDILVPLLQLLVLLLTLPLHLMALLGCWQPLCKSYFPYLMAVLT | |
| PKSNRKMESKKRELFSQIKGLTGASGKVALLELGCGTGANFQFYPPGCRVTCLDPNPH | |
| FEKFLTKSMAENRHLQYERFVVAPGEDMRQLADGSMDVVVCTLVLCSVQSPRKVLQEV | |
| RRVLRPGGVLFFWEHVAEPYGSWAFMWQQVFEPTWKHIGDGCCLTRETWKDLENAQFS | |
| EIQMERQPPPLKWLPVGPHIMGKAVK | |
| or | |
| (SEQ ID NO: 34) | |
| 1 | acttggaagg gtgaagggag gtgggcagat ggcatcaagt tgtaaatgcc agggaagtta |
| 61 | acgtggggaa ggggaaatct ggctgcttct ctaaccacgg cagctgttcc tcccttgccc |
| 121 | agtgctagga ctcagagttg actcaagttg ctctacaaag agctgggagg ggcggggtag |
| 181 | gataggggaa ttagtggaac atcccacccc ttcaacagct atttgagggc cagactggcc |
| 241 | agtgcctaaa cttgggcggg gtggggaagg aggaaactga tgttgataag aggtccatac |
| 301 | cccacattga ggtgatccac caaagcttct gtcaggcagg tcgctgcagt gatagcctct |
| 361 | cgcctcctct tttctgggga gagggggaaa aggaattgga ggggagactt ggttttttga |
| 421 | ggggtagtgt taaaggttcc ctacccatcc attactattc tgctagttgc cgaaatttct |
| 481 | taactaatct ctgaatgccg gagctgaaaa ggaccttgca aaacatagcg caacctcttt |
| 541 | cattttagag atgaggaaac tggtaccctg ggagggaaat ggactcactc agggacgcca |
| 601 | ggaagtcagc ggcagcgccc aggactcctg actcccaggc actctttctc cactactcgg |
| 661 | ccgcagaatc ggtaactccg agatttccag cccggactca ggaaacaagt gccccacctc |
| 721 | cgttccggtg gtttcaggcc tccaagaagc ccatctctgg ggggttcccc acaccctcct |
| 781 | ccctccagag tcgctccttc ccgccgccag gacgagaggc tcccagggcc aataggcagg |
| 841 | gcgctccggg gcaggggcgg ggctaaggtg cggcaggtcg gggattgggg gcgttgccga |
| 901 | ggggaccgga gtattgccca agggaccgga gccagttggc aagggaaagg agtttcaaaa |
| 961 | aaagcctctc tgaaattccc tctcccccat gcccgttgcc cgctagctgg cgtctttccc |
| 1021 | tggatatggt tagttactcg agatccaggc tccgggctga agctaggccg cggccgaaga |
| 1081 | gaaaacggcc gacaggatat ttggctcacc ctgcagctcc ttgcgttcat tctgcttggc |
| 1141 | ctggtgttct tttaggaggc gggacagcat ggtcacgtcg ggctgggggc tgggtacgcc |
| 1201 | gggccccctc cggctccgga agctggaacg ctcacctcgg ctccccgggg ccatgtcacg |
| 1261 | tgaccgctgt gtcacgtgag gttgggaacc gccctcttga gaccttgggt gaagatcttg |
| 1321 | gcggggaaga gaggtagagg gttgcggagg gcaggtgaag tgttctttat tgggagctta |
| 1381 | gtttttggtg agtgttggaa gaagtatttt ctaatactta tattgccaag ccagttttta |
| 1441 | acatttcctg tggatctcag gcttcgaggg ctggtggtgt caggccgtct atcaccgttt |
| 1501 | aattgaggga gcagaagctc cctggcctca tttaaattat tctccttccc tctcctcacc |
| 1561 | tcctggcagt gctagctggt tctcagttga ttcccagttc cttccctcaa ggagaggatg |
| 1621 | ctttcctgtc tctcccaacc ctctttcttc tcactacccc actgaagtct ctcagctctg |
| 1681 | ggtgatcatt gtagagaagc attttaggca cttcagacag cctcctaggg agcttggcta |
| 1741 | acttggcggg ctgtgtgaat gtagaatttc atctctgtcc ttttgtggtc aggaatgcga |
| 1801 | acccatttca gtgttagcag aagcgtaagt gctgtgccaa agtaaaaagt aatttatata |
| 1861 | aggcctctgt gaactaaaaa gaaatgcttc cgggattaac tccagcgaga acaattataa |
| 1921 | acatcatgag catattcttg ctactggtac aatctctctc ggtgcacctt gcactaacat |
| 1981 | gttgggtgac tttgggcaag ttgctcacca ttctaggtta tagttttctc tttagtaaaa |
| 2041 | ctaggagaag ggattggatt aaatcggtgg ttctcaaccc tggctaagca ttagaatcta |
| 2101 | aacattagaa tcacagaaat cacaaggctt ataaaaatgc cgtgattagc cgggtgcggt |
| 2161 | ggctcacgcc cgtaatccca gcactttggg aggccgaggc gggcggatca cgaggtcagg |
| 2221 | agttcgagac caggctgacc aacatggtga aaccttgtat ctactaaaaa tacaaaattt |
| 2281 | agccaggcgt agtggcacaa gcctgtaatc ccagctactc aggagcctga ggcaggagaa |
| 2341 | tcgcttgaac ctgggaggca gaggttgcag tgagccgaga tcacgccact gaactccagc |
| 2401 | ctgggcgaca gagtgggact ctgtctcaaa aaaaaaaaaa aaaaagcctt gattccagac |
| 2461 | taattaaata gaatctaagg agtgaagacc aaacatcact atatatatgt atgtatatat |
| 2521 | gtatgtatac gtatatatgt gtatatatgt atgtatacgt atatatgtgt atatatgtat |
| 2581 | gtatatgtat atatgtatat ataaaattat tattattttt gagacagagt ctcgctctgt |
| 2641 | cgcctaggct ggagtgcagt ggcatgatct cagctgactg caacctccac ctcccaggtt |
| 2701 | caagcgattc tcctgcctca gcctcctgag tagctgggat tacaggcatg tgccaccaca |
| 2761 | cctgactaat ttttgtattt ttttttttga gacggagtct cgctctgtcg cccaggctgg |
| 2821 | agtgcagtgg cgggatctcg gctcactgca agctccgcct cccgggttca cgccattctc |
| 2881 | ctgcctcagc ctcccaagta gctgggacta caggcgcccg ccactacgcc cggctaattt |
| 2941 | tttttttttg tatttttagt agagacgggg tttcaccgtt ttagccggga tggtctcgat |
| 3001 | ctcctgacct cgtgatccgc ccgcctcggc ctcccaaagt gctgggatta caggcgtgag |
| 3061 | ccaccgcgcc cggcctaatt tttgtatttt tagtagagat ggcatttcac cgtgttggtt |
| 3121 | agactggtct tgaactcctg acctcaggta atctgcccgc ctcggcctcc caaagtgctg |
| 3181 | ggattacagg tgtgagccac cgtgcctggc cttatcagta tattttaaaa gctgttctgg |
| 3241 | tgattctaat gtgtatccag ggttgaaact cactgggcta aaccatatct aaaactattt |
| 3301 | ccagctttgg cattctctca ttttgtgatt cattggcata gttgaagttg acgtcattat |
| 3361 | atttacttgc agggggagga gaaatgggtg ctgattgcat tagaagaaaa cattcttgaa |
| 3421 | ttttctgagt aagatttgag aattactgat caatgattca ttcaactatt tgcagaatag |
| 3481 | catcccaaat cctgggagat gtgcaactgt caaaggatta gtgaggagaa gcacatgaaa |
| 3541 | tgcttaagtg gcagcataaa gcaaaacatt ttaagtgcca aaataaaaat tctaataaaa |
| 3601 | acgttagata ctgtgtgtat gtgtctaaat tatatgtgta ctgtatcttc tttctaaaca |
| 3661 | tttcttagta ctcatcatag tcaagcactt tacatgtatg tattcattta atccctagaa |
| 3721 | cagttctgtg aaatattatt acgagtatcc caaatttgca gatgaagaat ctgaggtaca |
| 3781 | aggcagttaa gtaatagagt tcattagtgg tagagctagg atacaaatcc aggctatctg |
| 3841 | actccagagc ctgagtctta ccgctgtata ctgcataaac cacagttgag accccaggag |
| 3901 | ggtagaccca gagcactttt ctgggaatct ctctgtctct aatcattatg cccctctagc |
| 3961 | ttgctgggca tgagctgtag agaatcctgg caggggagga gtgtttgaga agaggctctg |
| 4021 | ctccagagaa gggctaatca atagaagtgg gaagagtgag agcatcacag agcagcctgg |
| 4081 | ctctgtcctc cccactctct ggctccagct tccaactgca gtgagcaagc aaaatagagg |
| 4141 | cagattggaa atgcctggtg cctcacctcc agagctgtgg ggcctggaag agaaggacat |
| 4201 | gggttggaag tgtcaagata atggtgacat gggataagtg aaacagaggg tgagggctaa |
| 4261 | tgggggttag tgttgggggg gtctcaaaag tgagtctaaa tgttaagatt aggccaactc |
| 4321 | agggagagtg tcaggcccat cctgtattac cctcaccctt cttggcacaa ctgcctgaca |
| 4381 | ttttcacaat cggtgtagag actgccagga ttctgcagac attagcctct cccttctctc |
| 4441 | ccacacactg gctgttcctt tgccctagag ctgagcagag gacatggggt atctgtatct |
| 4501 | agtgtcccca tctgatctct ctagggttat cctccatctg ttctttttct gggcctttat |
| 4561 | tgttcactgt ctaaccttag tttctgctgc agttgaagct cagcttcact tgtccttggt |
| 4621 | agagatggac acaagtcgca tacatatcta tctggagaat gactcttgaa atttgaagtc |
| 4681 | tctttctgct cacctctcct tacgatttcc tactcagatt attttccctt ggactgaatg |
| 4741 | aatgcaactc cacactcctt tcttcactca tttccctctt cttttgaggt ctctttcaga |
| 4801 | gttagcttta attagcaagc tccaaactga ctggctgggc caaacagggc tggaagctgc |
| 4861 | tgagttgtta ggggttggat gagggctgag ggaggacaca gagtgagtag acaaggcttc |
| 4921 | acatactggg agatctggct gggcttcaag agagggggtt ggtggtggtg tggctaagtt |
| 4981 | ccaaatgttg gcctgaagcc agtgctcaaa gaaagggggc ccttgagaca gtccaaatgg |
| 5041 | ctccctttgg taagtgacct cccagtcctt ctcaagtgtt aagggtttct tttggatcgt |
| 5101 | caaagaccgg agcggtcaag ctgtttcccc actgaattcc ctcaatcaca tttatgttct |
| 5161 | tttcctccca gccactccca tggttcaagc tttgactaca accagaattc agaggcaggc |
| 5221 | agaaggattc cagtgctgga gaggtgagtg aagtaaaaaa gttctcatgg tgtgcatgtt |
| 5281 | gggacggaaa agcctgacct tgggacataa gctccaaggc tctgttgcca gatgaggtgg |
| 5341 | agggagaagt tagccctgaa gtgtgtgttc tggaagtgtt tgcttgtaag ctagagacaa |
| 5401 | cagttgcaaa aagtgtgatt tgagggagct gaaaaatact gatctcaaag tggggaagaa |
| 5461 | gatgttgaaa agggaaggag ctggagaaag cctcagcttc cactcataca aaagctaaag |
| 5521 | ggctaaaatc ttggctggat ctggacattt ctcaacgtct aaaattttgg aaatttttat |
| 5581 | aaagattatt aatctttcat ttttacattt aatttattta aaaagttcag tttctcagtc |
| 5641 | atactagcca catttctttt tctttttctt tttttttgag acagagtctc actctgttgc |
| 5701 | ccaggctgaa gtacagtgta ttaatctatt ccatggagtg gagtggataa tctattccat |
| 5761 | ggattatcat tttactttgt tagtggtatc cttagaagca caaaattttt aaattttttt |
| 5821 | ttttttttga ggcagagtct cactgtgtcc cccaggctgg agtgcagtgg tgctatctct |
| 5881 | gctcactgca ggctccgcct tctgggttca agcgattctt gtgcctcagc ctcctgagta |
| 5941 | gctgggatta caggtgtgta ccaccacgcc cagctgattt ttgtattttt agtagagatg |
| 6001 | ggtttttgtc atattggcca ggctggtctc aaactcctga cctcaagtga tccacctccc |
| 6061 | tccctcccaa agtgctggga ttaccatgtc tggcttggaa attattttga aataattata |
| 6121 | gatcagagga agttgtaaaa atagcacatg aagtcttgtg tacctttcac tcagtttccc |
| 6181 | ctaatggtga catcttatgt aactgtagca taaaatcaaa accaaaaagt tgatattggt |
| 6241 | acagtattgt taactggcct gcagacctca ctcagttttc accatttttt acatgcattt |
| 6301 | atttgtttgt ttgtagttct gtgcagtttt atatcttgta tagatttgga taatcaccac |
| 6361 | cacaatcaag atacaaaacc catcaccaca aaggaacccc cttgtgctat tcctttatgt |
| 6421 | ttgtccccac ccccctccat ccttgtcccc tggcagccag taatctggtc ttcatttcta |
| 6481 | tagttttgtc attttgagaa tggtatgcga gtggaataat acagtttcag cattttttgt |
| 6541 | ttggagacag ggtctcactc tatcacccag gctggagtgc agtggcaaga tcatggctca |
| 6601 | ctgcagcctt cacctcctgg gctcaagtga cactcccgcc tagcctcctg agtagctggg |
| 6661 | accacagatt tggctaactt ttctattttt tgtagagatg ggggtctccc tatgttgccc |
| 6721 | aggctggtct ccaactcctg ggctcaagtg atcctcctgc cttggcctct caaagtgctg |
| 6781 | ggattgcagg catgagccac tgtgcccagc tcagcattaa tttttaattt aactaattcc |
| 6841 | taagctcttg actgaaatac aagaagttct ctaacagttt atttatttta atattgagct |
| 6901 | taccgcattc tctggatcct tctagtttct tttttttttc tttttttctg atgtggagtc |
| 6961 | tctgtcaccc aggctggagt gcagtggtgc catctcagct cactgcaacc tccgtctcct |
| 7021 | gggtttaagt gattcttgtg cctcagtctc tggagtagct gggattacag gtacccgcca |
| 7081 | ccacacccgg ctaatttttg tatttttagt agagacaggg tttcaccgtg ttggtccggc |
| 7141 | tggtcttgaa cttctgatct caggtgatcc gcctgcttcg gcctcccaaa gtgctgggat |
| 7201 | tataggcgtg agccaccgcg cccggccccg tctagtttct taatttccct cttcacctac |
| 7261 | gatattatct tccactccaa cattctggtc tcatttctcc ttgagagaaa tctacatgtc |
| 7321 | taaatttact aggctggtct agcacgctct tgtgtgttcc cctccctcct ttgcccctct |
| 7381 | atttatagcc aggctaattt tgggtggcct ctctctctct tctttcctga tctttcctcc |
| 7441 | tgtggtggtg aggtgacttc tcaaatattt ggagagagga ggtcagaagc agattcttgg |
| 7501 | catctgattt cagccctgga tcacagaagc cagtggagtg ggaatggaga caggcagaag |
| 7561 | ctgcaggtgc agataggagg cagcttgggc tctaaaggca ttttgagctg ggtcgggggc |
| 7621 | ggggggacct gggcagggag tcagtagtcc cagttctgtc ctaattttgc aattctgcat |
| 7681 | tcccatgtca gctcttctct actgtctggg gctctgagat attaaaaagg atggggaggg |
| 7741 | catggtgaaa gtagaatcct ggtgccagcc ctgctgacag catatgtatt tccttatagt |
| 7801 | acctgtttag agatgtgtta gtgctctgga ggggatagcc acaggtgtag tattggaaaa |
| 7861 | cagagggcca gacttccaaa tgtctgttaa cttatccaag gcaaagactg tcccagggca |
| 7921 | gcagagtaag aacccacttt ttttttgttt tcaaagaagt ataatcctga acaatgaagt |
| 7981 | aggaaagaca gaacacagga agaggaagga ggtaggacac ttattggaac ttttaagaaa |
| 8041 | gggaaagaga agaaagaatc gtaagaatat gatagtgttt gaagggcaga gacaacacta |
| 8101 | gaaacattga gaaatactct gagaaagatt ccaagtgtgg cagagacaag aatgatgaca |
| 8161 | aaatagaatt tgggatgaga caaaatcaga tagtgagaga gagaagggaa gatggacaga |
| 8221 | tgtatattca caagaccaac accagtaagc aaggggagta ggaaggggaa gtgggagcat |
| 8281 | tcgaggttcc cattatgcca aattatttcc tgtctctcct tctggcccca tttctgtatc |
| 8341 | ggagttataa atagcagaga gttggaaagt gtccccccac ccccttgcct ctgtcccagc |
| 8401 | ctgagggaaa gggagaggaa gagggacagg ccaatgggtc cctgtggaga tcccatctca |
| 8461 | gccccaccca ggtcctgctg agccagtcca ggactctgcc ccctcccatc ccctttcatg |
| 8521 | gataggaaat gtgcagtcct gggacgggtc tggtagctgg ggacaccctt tacatccctc |
| 8581 | tgcctcttgg gtccagtctc tttcatcttt gccttctttg acacccactc ccctccccac |
| 8641 | tgcttaattt cctcttcctg taatcatccc cagtcgtttt cttttctccc ttcattccat |
| 8701 | cccttgtcaa ttaatctctt gcccttcttt cttcctctct attcctttcc tttttccatt |
| 8761 | tctccatttg ctccccgtat ctcccgagtt tctctctctc ttcttgcctc tttttctctg |
| 8821 | ttcccttgaa tcctgacgat gtggctagca ctgctgtggt cattgccggg ctgggggcgg |
| 8881 | gggatgggat aggatggggg agggcagcgg tctgatccca acagcagaaa gagtgctcta |
| 8941 | tgtgaccatg ggggaacagg gagcactaag atgccacgct gcacccaggc ccaggacggc |
| 9001 | tcccctttca tttcctctct atctgcacat ctctcttccc aggttgtctt ttagcgtctt |
| 9061 | cccaacttct catctcttac cctccttcct ctgtttcagc ccctctcttt ctatctgtac |
| 9121 | ttctctccct ccgcattcca aggcgccgcc tccaccactc ccggggtggg gatggggttg |
| 9181 | ggggagaagg ggaggagagc gccgcgcagg ggcggagccg gagacggtgc tgggcttggg |
| 9241 | gggcgtggtg gtggggggtc agcaaggcta gtttccatcc cagccaccag cctgggcatc |
| 9301 | cccttggaga cgggcttggg tctccacctg ccgcgggagc gaggggcggg gccggaggcg |
| 9361 | gggcctgagt ggcgtccccg ggagaggagg cgggagccgg agtgggcgcc ggagctgcgg |
| 9421 | ctgctgtagt tgtcctagcc ggtgctgggg cggcggggtg gcggagcggc gggcgggcgg |
| 9481 | gagggctggc ggggcgaacg tctgggagac gtctgaaaga ccaacgagac tttggagacc |
| 9541 | agagacgcgc ctggggggac ctggggcttg gggcgtgcga gatttccctt gcattcgctg |
| 9601 | ggagctcgcg cagggatcgt cccatggccg gggctcggag ccgcgaccct tggggggcct |
| 9661 | ccgggatttg ctaccttttt ggctccctgc tcgtcgaact gctcttctca cgggctgtcg |
| 9721 | ccttcaatct ggacgtgatg ggtgccttgc gcaaggaggg cgagccaggc agcctcttcg |
| 9781 | gcttctctgt ggccctgcac cggcagttgc agccccgacc ccagagctgg tgagtcaccg |
| 9841 | cacccgccca gagtcgccat gcccgagcca cagatcgtcc ccctccccac tctgtgggcc |
| 9901 | tcctcatttc tctgttttct agccccacca agacctagac tgcccacaga catcccacat |
| 9961 | cccaacctgg agccttgcct catctggctt gcgtctgaag ctgcacttcc cggccctgag |
| 10021 | accagtattt tgctttaggg atgagttgga aagcaaggtt cttgtcttgg cagcgaacca |
| 10081 | tctccttctt ctgggccttt cccccaactt gcatccttga tccagcccca gggcctctgg |
| 10141 | ctcccctgct tcttccaagg gctgaattcc ccaagggagg gagactgtct gtctctgctt |
| 10201 | agaatgggag gagatggaaa ggacatagaa gttgagggtg ccatgagagg gatgcatgca |
| 10261 | gggcagactc cagaaataac ttcctgctag agcattgcca tggatggaat gagggcagca |
| 10321 | agggcactgg aggccaggag agagcttcca cttctgtggc ttaagaccac gggaagattg |
| 10381 | ggagaggatc tgcaggtctg ccaacctgca gtaggtggct tggtgataga gagtggcagc |
| 10441 | aaactgaacc ctcaaagtac tagtagcagt agtagtagcc gcagcagctg tagcagtgag |
| 10501 | agagatccag gaaggatgct ggccaggctg ctccccttcc tcctccttag caaatttcca |
| 10561 | actccaggaa tctcagcagc tgggaagggc caggaggagt aaggggtgga ggacaattct |
| 10621 | aattttttct aatcagttca ggacccatgg gagatggata tacttttgtg aggggcctgt |
| 10681 | gactggtcat gttgcctgta tccttggctc ttgctacatg tctgattgta aaaagggagg |
| 10741 | ccagaggtga agaaagcttc tcacctgctc ctgctagggg gcttttctct cttcaaccag |
| 10801 | tgcctaagcc acattaagta tccattactg ggatcaatgc tgtccactgg gactgtcttc |
| 10861 | tgcctctact gtcggtctgg gggcaggggg cagggacaag agctcatttc tcctcacttg |
| 10921 | cttggggagt gggggcctag ctctaatctt tcttcttcca ttatccctat catctggtag |
| 10981 | cagggtcggt ggtccccaaa actttgggag agatagaaag caacggactt catctcctct |
| 11041 | tctgtttacc atctgcttcc tcattcacct ttgctccctc cctcccttcc tccctccttc |
| 11101 | tccatctgtc agagttcgag gactggaggc ctttttagga catgctgaac tctctaagct |
| 11161 | atttccaggc aaattctagg ttatttttaa tagcttggtc tcttgtcatt tccccctcct |
| 11221 | ctctgaaggt ggcccctggt tccgtctccc agagccaagc tggggccttt cccagagggc |
| 11281 | ctgactgcct caccctgctt ttgctccagc agggggtgct ctgctgctgg ggggcggggg |
| 11341 | gtatgtgaga ggccaggcac ctgctcagtc cctagctttt gagttgcagg tggcctgcct |
| 11401 | tagcactcac tgatgaaaaa aacttcttgc ctgttttgat gtcttttagt ctagctctgg |
| 11461 | gatgagactt taaggtctaa cctttgctgt gtggttccag cctcatttac ttccctcaac |
| 11521 | tgtaaaaagg atataaacat agtattacta catagggttg ttgtgcagat taagagttct |
| 11581 | taatatatat aaaatgctta gaatagtgca tagcccgcag tgagtgctgt gaagtgttag |
| 11641 | tagtattgct attcttgtat tgtgattcac agagcgcctt acagagattc tggatccaaa |
| 11701 | ggcttggcta gagggcctcc ctggctgagc cagccttcca ggccaagcat cctccccaga |
| 11761 | gggccaccca gattgagagg ggccaaagag gggctggact tgggctgggg ccctggagtg |
| 11821 | tgtggagaat cgagaagtgc agtggtcgtg ggctactcct ttgtcttcac ttagctgagc |
| 11881 | tcccaggggg tccctctgcc ccccagctgc caacactttt tttttttttt ttttgctttt |
| 11941 | ctctctgcag tggctacact gtggctgtcc agaagactgg ggtggttagg gcgtatggca |
| 12001 | tgaagccagg aaggagtgtg tgtggctgga ccagaggtgg agggactaga gaggatgctg |
| 12061 | ctgggtgctc ttgttccact aaggatcgat tggtctcttc tccaccaaga gcggactggg |
| 12121 | catatctatg cactcagctt ctttcttcca catgggcccc tcccctcctc cctacttttg |
| 12181 | gcctccagag gagatgtgaa catagaacaa ggataactta tctgggtgct tagctatgca |
| 12241 | ctgaccagct gtgacactgg gtatctctat gagtccacaa aatgtgtgtg ttcagtaaac |
| 12301 | acattctgac actccctatg gggcaagcac aaagatgaaa agacagcccc gacactcaga |
| 12361 | gagatggcca cttctatgtt tggaggctgg gggtactgct gacttgcctg aaggttgcca |
| 12421 | tttatttatg cagggctgta tcaccccgtt tccttttctg cccagggtac cctcatctcc |
| 12481 | ccactctctc cttccctttc tggggtggtc tcagtgttct agagacaggt cagtcactgg |
| 12541 | gtggagtgac aaagtgttgg agttaggccc atgtggattt gaattccagc atcactgctt |
| 12601 | aatgtctttg agtgagtttt ctcatctgaa agacaagaaa agaatcctta tctcatagga |
| 12661 | ttgttctgat gattaaatga cataatgcat gtgacttgcc tatcctggtg cttggcacat |
| 12721 | atgtggacag tgatgaatgt tagtttctta tatccctggt gtctagcctc ggatctgacg |
| 12781 | tcatagtagg tgctgaataa atatgatttc cttgtctcac cagcgcctga cacagggctt |
| 12841 | ggcatacaat agactctcaa taagtagttg aatgccaaat gtgtcttctc ttctctacta |
| 12901 | ctccctatac cccttctctg tcttgacact ggctctgaca agggatggca gctgctaaga |
| 12961 | gatgaggagg agttgtggga aggaagaatg gctctctgcc ctccccctcc accccatcag |
| 13021 | agctggcaca gtgccccaca gatgcctgtc tgtaatactg cctaacatgg ttttgggcct |
| 13081 | tgccccccag gaagggagat ggaggagaag agtgtgggag agaggcgttg aggtttgtcc |
| 13141 | cactgccact tctgagtctc tccttctgca aagagaggac ccatggagcc agctgggtgt |
| 13201 | cagtcatctt acctcacccc cgccttcact ctggcttggg ggttcagccc caggggaccc |
| 13261 | aggcagcctc cattcccagc actgtgctcc cctggggaag acggcttggc tgtgatcatg |
| 13321 | gaaaattgtc ctgccaagaa agttgtagct gggaaagagg ctgaggggga ggcaggagag |
| 13381 | aagactgggt gggggtggaa gggaaggaga aatcatggac atggggagaa ggaaggatgg |
| 13441 | ggaaggggat tcaggatgtc gggaagagaa tggggtagca ttggaggcag aaggagaaac |
| 13501 | ttgtccctac ctccatgcca gccagagtga ctgatggaat cctgggctgg cacagcttct |
| 13561 | gggaggtggg gtctttgctg ggtccctgat gagggggcag tgggtccgta tctagcctct |
| 13621 | tgcctggcct ctgaagctgg tccctgagcc acactctgat gccagtctgg ggccctgtta |
| 13681 | cttttgctcc cagcattctt ggcatttctg gctgggtttc aactggactg ggttggggag |
| 13741 | cagggcagag cttggggatg gggccaagga ggggataggg aaggcctact caggaacagg |
| 13801 | tgctgggaac aggcagttct ttcaaaccag cactgttggc ctggctgctt gggttggcgt |
| 13861 | gtatgtgtgt gtgtgtgtgt gtgtgtgtat ctactgtgta tgttgatccc ttatccagat |
| 13921 | agtatgtaca tgcaacgtga tgactgcatg accaagcata ttaatttgtc cttgccaggg |
| 13981 | tttgagaaaa ctgacatttg ccccttctct ttagtccttg aacactctct ttagtactga |
| 14041 | ggggttgggc ctgggcagct ctaatgagat tgggtcattc tgacctctaa ctcctgtccc |
| 14101 | tgtccctgcc cctgccccat cttgcaggct gctggtgggt gctccccagg ccctggctct |
| 14161 | tcctgggcag caggcgaatc gcactggagg cctcttcgct tgcccgttga gcctggagga |
| 14221 | gactgactgc tacagagtgg acatcgacca gggaggtgtg gccctgcatg aacagagtgg |
| 14281 | gggaagcgtg tgagcgggga ggagaggact tgggctcctc ttccctcccc taattcccag |
| 14341 | tgtcctgcct ctagctgata tgcaaaagga aagcaaggag aaccagtggt tgggagtcag |
| 14401 | tgttcggagc caggggcctg ggggcaagat tgttgtgagt attgcttctc atgactgaat |
| 14461 | gcacggatgg ggtgtgtgtg tgtgtgtgtt tatggtgtgt gcatacgcat aggtgtgctt |
| 14521 | agagaacaca agttaggaat atggtatgat tccaagtaca tcagggagat ataaaaaggt |
| 14581 | gtgagacatg gtccttgtcc ttataaatgt aaaaatgtct gtccattcat tcatccatcc |
| 14641 | atttgtcaaa ctcttactga gaacctttta agcatcaggc attgtgctag ttactacagg |
| 14701 | ggaaggctca tgcctgtaat cccagcactt tcggaggccg aggcaggtgg atcacctgag |
| 14761 | gtcaggagtt cgagaccagc cggaccaaca tggcgaaacc ctgtctctcc taaaaataca |
| 14821 | aaaaaattag ccgggcgtgg ccgggtgcgg tggctcacgc ttgtaatccc agcactttgg |
| 14881 | gaggccgagg tgggtggatc acgaggtaag gagatcgaga ccatggtgaa accccgtctc |
| 14941 | tactaaaaac acaaaaaatt agccgggcgt ggtggcgggc gcctgtagtc ccagctactc |
| 15001 | agagaggctg aggcaggaga atggcgtgaa ccggagaggc ggagcttgca gtgagctgag |
| 15061 | atcgcgccac tgcgctccag cctgggtgac agagcgagac tccgtctcaa aaaaaaaaaa |
| 15121 | aaattagcta ggtgtggtgg caggcgcctg taatcccagg tactcgggag gctgaggtag |
| 15181 | gagaatcact tgaacctggg tggaggaggt tgcagtgagc tgagatcgca ccattgtacc |
| 15241 | ctagcctggg agacaagagc aaagttccgt ctcaaaacca accaaacaaa caaacgaaaa |
| 15301 | aaccagagct ctctgtttct ctctctctct ctatctttca gtaacacgca tagatacaca |
| 15361 | attaccaata cagatcactg tggggcagaa tctggttcat gttaagtgag tggtctagtc |
| 15421 | tccagtctat aaaagtccaa aggaggagta gagagaagac ttctgcagag gggatgattt |
| 15481 | gagccaggct ttaataatag gtaataccta gcctgtgcaa catagtggga cctcatcttt |
| 15541 | ataaaaaata aaaacaaatt agccagtcat ggtggtgcat gcctgtagtc ccagctacac |
| 15601 | aggaggctga ggtgggagga tcacttgagc cctggaggtc gaggctgcag tgagccatga |
| 15661 | ttgtgccact gcactccagc ctgggtgaca gagtgagacc ttgcttcaaa aaaaaaaaaa |
| 15721 | aagtaatact tggagagtga agcggacagg aagttctttg cagatgagat ggtgacactt |
| 15781 | acaaaggtcc agggacaggg ccaagcttgg cattttggag gactgtgaca tgatcaggga |
| 15841 | gacacacatc ctatgtggtg gcttaattgt gtcttttggc tccaggcaga atgtggaaca |
| 15901 | aggagatctc catttgaggg caaggaagtg ggtgcagaca ggttgctggg ttatgcatgg |
| 15961 | acctgtgtaa cactggcagg gtaatggtgc ttgagtggtg ccggcatagg ggtgtgtgtg |
| 16021 | tatgtgtgca tgtgcatgtg catgtgagca cacatgtatc agtatctgcc aaatctctgc |
| 16081 | atatgggcag catgcctcaa gcaggtccct ggcccacaga gtgaaatgat ccccatccct |
| 16141 | tcctccccca gacctgtgca caccgatatg aggcaaggca gcgagtggac cagatcctgg |
| 16201 | agacgcggga tatgattggt cgctgctttg tgctcagcca ggacctggcc atccgggatg |
| 16261 | agttggatgg tggggaatgg aagttctgtg agggacgccc ccaaggccat gaacaatttg |
| 16321 | ggttctgcca gcagggcaca gctgccgcct tctcccctga tagccactac ctcctctttg |
| 16381 | gggccccagg aacctataat tggaagggtg agtcactcct cgggaagggg agaaggggac |
| 16441 | caaaacctcc tcttacctca gagacagggt tggggatggc acatggccaa gcatgaccac |
| 16501 | atgtgcactg ctgtatggcc ccagggcact gccatgcctt ccaccccatt gagctagtgc |
| 16561 | acacatgaat ggggggtgcc tcctttccct cgcacggcca agtgttcctc aacatgctgg |
| 16621 | catgggcccc aagtgcacgc tgggcctgca gctggggcct gcatgctcca acacactagc |
| 16681 | ccacacctca tcactgccat tcccgtctcc gcacgctgct gctggctgag ctgacactcg |
| 16741 | gtgagtgtga tgccacatct gggggacccc aggaagcctg ggttggggac agggtgggga |
| 16801 | gagggctaga aagaagaggc agggcttccc cgtgtgcctg tctaactcag tgtccggcct |
| 16861 | gaggggtgtt ccttgcgccc tgccctgggc actaacaggt ctgtccttgc aggcacggcc |
| 16921 | agggtggagc tctgtgcaca gggctcagcg gacctggcac acctggacga cggtccctac |
| 16981 | gaggcggggg gagagaagga gcaggacccc cgcctcatcc cggtccctgc caacagctac |
| 17041 | tttggtaggg acctctcccc ggcccagaac tgctctaacc ctctgctcct ctctcttgtc |
| 17101 | ctctctctcc atgctcccat ccttctgtct ctgtttctgt ctctcacctt gtctctctct |
| 17161 | gtctttctgt ctctggctgt gatctctctg gtctcttttt ctctctccac ctcttcttct |
| 17221 | tccaccattt tctggccttt ctgtggctct gtctccctac tctgtggccc ctactctgga |
| 17281 | tgtcccctcc ctggtgtctc accccacccc ccacagggtt gctttttgtg accaacattg |
| 17341 | atagctcaga ccccgaccag ctggtgtata aaactttgga ccctgctgac cggctcccag |
| 17401 | gaccagccgg agacttggcc ctcaatagct acttaggttt gtaagctccc acctcctgga |
| 17461 | ctctaggggc atggcccagc ctcccctcct tccccaggga actcgacctt tggtgcctta |
| 17521 | taatctcctc ctcccccaac acacacccag ggagacatac attgggccca aattgcagag |
| 17581 | aagagctggg tccaatgatc aggcctaaga ggaggaggcc cccagggtgg tggcctctgg |
| 17641 | ggctgtgagc caggggtctc catggaggaa gattcaggtg gaatgagagg gccagggctg |
| 17701 | aggatatttt gggaaggaca gtcctgtctt ctagggggac tttccctgag gggatggatg |
| 17761 | gtgggcacat attgaagaaa gggctaatgt tgttggtaag tccctctcgt tgtctcatct |
| 17821 | gcattcctct gcagaggagg aggaaaccag gcctgggaga tgtttgggtg aagcaggcgc |
| 17881 | tctctcactc ccccttgtct ccccctcatc catgtgaaga cttcccctcc ctgccaggat |
| 17941 | gagggagttg ggggaaagag gtgcactggg tgggattcgg gcctgagagg gacctctagc |
| 18001 | tcttctagct ccctgggtgt gggcagggtg aggccactgt gctcagcctc ctacctgggc |
| 18061 | tcctggcctt ctcagccatc acctttctct ctcttgccca gtccctgagg ctgacctcac |
| 18121 | tgcacttttt gtgccaagct tgtctctggg cctggtgggt gtgggaggct gccaggccct |
| 18181 | gtggggagga agagctatcc agctgtggtg ctgatgactt ggggggacct atcttttggc |
| 18241 | tcttaaccta ggggaggggg cagggtgcag gggagctgtg acttggctct taacctgtag |
| 18301 | ggagggggca ggggctgggg gagctgtgac acaccccagc ttctgagtct tggggtgaag |
| 18361 | acttaggggt aagtcaccct tcccccaggc ttctctattg actcggggaa aggtctggtg |
| 18421 | cgtgcagaag agctgagctt tgtggctgga gccccccgcg ccaaccacaa gggtgctgtg |
| 18481 | gtcatcctgc gcaaggacag cgccagtcgc ctggtgcccg aggttatgct gtctggggag |
| 18541 | cgcctgacct ccggctttgg ctactcactg gctgtggctg acctcaacag tgatgggtga |
| 18601 | gtgggtagag ggccgtgcca cctgagggag gctgggtcta gtagccccag tctggctgag |
| 18661 | gccacttagc ctcctgctgg ctcctctggc cagggaggac ccacactgaa tgtttccctc |
| 18721 | tctccatagc tggccagacc tgatagtggg tgccccctac ttctttgagc gccaagaaga |
| 18781 | gctggggggt gctgtgtatg tgtacttgaa ccaggggggt cactgggctg ggatctcccc |
| 18841 | tctccggctc tgcggctccc ctgactccat gttcgggatc agcctggctg tcctggggga |
| 18901 | cctcaaccaa gatggctttc caggtgtgac ggggaactgg aaaggctcag ggagggaggg |
| 18961 | gccacaggag ggatggggaa gcccctcaga ggtcagggtg tggtcttctg aggactcagg |
| 19021 | gagagagggt ccctgagctt atgtctgagc tgtaccattt accagctttc tgaccttggc |
| 19081 | aagttcctaa cctttttgcg ttagtaatat ctgcagggag tggccaagag gattaaagat |
| 19141 | gatgtatgta gagtgcctgg gattttgtag cctctcaata aaatagaaaa catacctgag |
| 19201 | tgactggggg gagttgaggc ctggatcttg tctgcaaggc ccccagccag cgtgactgcc |
| 19261 | ttttccctgt gccctgcaga tattgcagtg ggtgccccct ttgatggtga tgggaaagtc |
| 19321 | ttcatctacc atgggagcag cctgggggtt gtcgccaaac cttcacaggt gaggggagtc |
| 19381 | gctgggatga gggaatgggt gtgggtggaa tcagcagagg catcagggga ggcagaggcc |
| 19441 | tgcgggaggt gggattgagg gaggctgaca gctggttctc taggtgctgg agggcgaggc |
| 19501 | tgtgggcatc aagagcttcg gctactccct gtcaggcagc ttggatatgg atgggaacca |
| 19561 | ataccctgac ctgctggtgg gctccctggc tgacaccgca gtgctcttca ggtgagcccc |
| 19621 | tctcaacctt ttccctccct gaggccgtca gcccctccct gtgactctga ccccgacctc |
| 19681 | agtgccaaat ctaatgctga agagtgtttc ccagcctcat gttctcatgt ttcttgtgct |
| 19741 | cttgactccc caatcccagg gccagaccca tcctccatgt ctcccatgag gtctctattg |
| 19801 | ctccacgaag catcgacctg gagcagccca actgtgctgg cggccactcg gtctggtgag |
| 19861 | gtgggatcgg gtggcacctg gaccctggca gcttcccctg ccctcccttt gatcctttat |
| 19921 | ctccccagtt tggggctggg gctgtgacag gatgtgacag atggggtggg ggtaggggcc |
| 19981 | ttggcccatc agcctcgttt ggctcaggag ccacctttgc ccccgcagtg tggacctaag |
| 20041 | ggtctgtttc agctacattg cagtccccag cagctatagc cctactgtgg gtgagtgcgg |
| 20101 | tcccccctct gtggctcgcc cttttgggtt tcccagggag gggggtcact tcgaggtggt |
| 20161 | agaagagcac ccttggaatg gggtgagctg gagcagctct gcagctcagc agctcctcct |
| 20221 | ttcccaacat gccacagccc tggactatgt gttagatgcg gacacagacc ggaggctccg |
| 20281 | gggccaggtt ccccgtgtga cgttcctgag ccgtaacctg gaagaaccca agcaccaggc |
| 20341 | ctcgggcacc gtgtggctga agcaccagca tgaccgagtc tgtggagacg ccatgttcca |
| 20401 | gctccaggtg gacactgacc ccttggcttc tgagggtcat tttcatggct ccatctcttt |
| 20461 | tccctgattc ctcttagctg ctttttcccg agcacactcg tgcctccttc taagacctag |
| 20521 | acacgtggga aacctgtctt ctgagctcac ttcctcctca tctgctgccc tctcctgtca |
| 20581 | tttctgcact ccctggagga ggaggaggtg caaggggctt catgtcccct cttccagctg |
| 20641 | aatggaggag gtggcagctt ttatcatcac atctggtcct caaagcccta aaggtttccc |
| 20701 | ctgtccccta ccccagtctt tctccctcct ccttgctcag ttctccacct ctctcattca |
| 20761 | tctccctaat attctttcca agttttagaa ttggtcctag gtctggctgg ggcactgagc |
| 20821 | agggctgagg cctggggatt gttccagtga ggaatagacc ttcctcttcc taggaaaatg |
| 20881 | tcaaagacaa gcttcgggcc attgtagtga ccttgtccta cagtctccag acccctcggc |
| 20941 | tccggcgaca ggctcctggc caggggctgc ctccagtggc ccccatcctc aatgcccacc |
| 21001 | agcccagcac ccagcgggca gaggtgagca tgggttctgg tttagcccag ggggaggagc |
| 21061 | tgggagggca aagatcatgg tccctcccca gtgacaccaa ttcacagctc acagagccct |
| 21121 | ttcacgtatg ctactccagt aattcctcat atctctaggt gcctaaatga ccatgttcct |
| 21181 | agtcaagggg acagagctcc tctactccat gtagtcattc agggggctca ggttgatggg |
| 21241 | attctgtaat cttcaaatgc ggccttcaag ctcttgaagt cacattcatc ccattcagct |
| 21301 | ggaagggaga acatgaggcc ccttgcttgc atggttttta tggctggccc agaagtggtg |
| 21361 | cccttgacat cttttcccgt tctaaatccg gaactcagtt acatgcatgt gagctggggc |
| 21421 | agtgtggccc agctcttgac ttgctgctag tctctaccat ggcatggtag tgctcccatc |
| 21481 | actaccatga tgagaggacc cagaggagag gacccagttc aggatcacat ggctaataag |
| 21541 | cagcagatct gagagttgag gacaggttgt ttaatcctgt gatactctaa cccaccatat |
| 21601 | gccatggtga caggtttgcc ccctgccttg ccccctagga atgcccctta tctcatgtct |
| 21661 | ctccccagat ccacttcctg aagcaaggct gtggtgaaga caagatctgc cagagcaatc |
| 21721 | tgcagctggt ccgcgcccgc ttctgtaccc gggtcagcga cacggaattc caacctctgc |
| 21781 | ccatgtgagg ggggcagaga gcagggtggg ggtgggaggg caagagttca aggattgaga |
| 21841 | gaaagccctc tcaggaggac cagtcagagg gaagggctga gcctgcagaa aaggcagaag |
| 21901 | gtggaagagg acctgccgat ggacttggag actgagatga gggtcagtga tggggccacg |
| 21961 | ggggctccca tggcaaggaa tagcaatcct tcacagtaca ctaataatat gcagtctaca |
| 22021 | tacatctcat tggttagtct tcaaacctct gtgaggtagg aattatatta gtctcatttt |
| 22081 | atagatggag aactgaagcc tagagaggtt aagttacttg gccaaggtaa gtaatggtgg |
| 22141 | agctgagatg agaacggagg tcccctgact cccagtcgtg tgtacagagg cctgaggctt |
| 22201 | ggacgggtgt gatggggacc tggggtggga agagaggctc agggagtgaa ggcaggtact |
| 22261 | gggggagcag actgggcggg ggaatggtag ggggaggtgt tcagaactta gcaggagtgg |
| 22321 | ggtctgactc tccagggatg tggatggaac aacagccctg tttgcactga gtgggcagcc |
| 22381 | agtcattggc ctggagctga tggtcaccaa cctgccatcg gacccagccc agccccaggc |
| 22441 | tgatggggat gatgcccatg aagcccagct cctggtcatg cttcctgact cactgcacta |
| 22501 | ctcaggggtc cgggccctgg accctgcggt gaggacctgg gggcaggatg gggtggggtc |
| 22561 | ttgaggggct ccagtaaccc agactgacct tgccttctct cccattccag gagaagccac |
| 22621 | tctgcctgtc caatgagaat gcctcccatg ttgagtgtga gctggggaac cccatgaaga |
| 22681 | gaggtgccca ggttggcaca tctgccctta tccctacgtg agtagcctct ccataagccc |
| 22741 | gtagaccacc cctgactcta atctctttcc acccttggcc taggtcacct tctacctcat |
| 22801 | ccttagcacc tccgggatca gcattgagac cacggaactg gaggtagagc tgctgttggc |
| 22861 | cacgtaagcc aggcggggcc ggaagggtga ggtgggggta gtcatattga cctcatctga |
| 22921 | ccccttggga ggtgcctgtg cctgatgccc atacttgccc ctgcccactc accaggatca |
| 22981 | gtgagcagga gctgcatcca gtctctgcac gagcccgtgt cttcattgag ctgccactgt |
| 23041 | ccattgcagg gtgagcctgg cccaaggggg cacctccatt ggagggaggg ggcattgaca |
| 23101 | tttccaaacc ttggccaggg ccctgccttc attgagccag gccccagacc ttctcacccc |
| 23161 | tgttctgacc tctccacgcc agaatggcca ttccccagca actcttcttc tctggtgtgg |
| 23221 | tgaggggcga gagagccatg cagtctgagc gggatgtggg cagcaaggtc aagtatgagg |
| 23281 | tcacggtaag tgtcagggat gaggcccctc catggtcacc ctccctcctt ggcacagagg |
| 23341 | agaggctgag ctgtgtcccc agggctgggg ctcttctacc atgtggcagc atgcagtttg |
| 23401 | aggcctctgt cctgcatatg gaccttggct gtgggaaggt gttcctgctg gggcctcatt |
| 23461 | tgaccatttc ctggtcattc tgtctggctg tctcacctgc tggtgtggta ggccatgaga |
| 23521 | gtccagggaa gcttctctgc tgtgggcctc agctggggga tgggaacatg gggcgggggg |
| 23581 | ttaatgtctc tccctagaac tctgcctttg cttggctggg gcctctcctc acaccttcca |
| 23641 | gggacacttc cagggtttcc cggatttggt gggtggagag aatggtgcct ggtggggtct |
| 23701 | aggatcctga gtatttgttg aaattgtgtt tcctgagggc cttctcttca gcccttgttc |
| 23761 | cttccatctc tgtggattta ggatccactt gaaagctgag ttccttggct gggcacggtg |
| 23821 | gctcatgcct gtaatcacag cactacagga ggccaaggtg gcaggatcac ttgagcccag |
| 23881 | gagttagaga caagcctggg caacatagat cctgtctcta caaaaaatta aaaaaaaaaa |
| 23941 | aaaaagaaaa actgaggtct ctcccctcat actctctttt tcccacaggt ttccaaccaa |
| 24001 | ggccagtcgc tcagaaccct gggctctgcc ttcctcaaca tcatgtggcc tcatgagatt |
| 24061 | gccaatggga agtggttgct gtacccaatg caggttgagc tggagggcgg gcaggggcct |
| 24121 | gggcagaaag ggctttgctc tcccaggccc aacatcctcc acctggtgag gcttaggtgg |
| 24181 | gggtgggggt tagatgtggg gacagatgtt atggggagta agggtagtga gtgcagtgat |
| 24241 | ggggcaggag gaaggcggtg gggaggagat tcgatcttag cactgctgtg attggaggga |
| 24301 | cactcactaa gacccccctt ccgtgtccag gatgtggaca gtagggatag gaggcggcgg |
| 24361 | gagctggagc cacctgagca gcaggagcct ggtgagcggc aggagcccag catgtcctgg |
| 24421 | tggccagtgt cctctgctga gaagaagaaa aacatcaccc tggtgagggc aggccagact |
| 24481 | cggtgcagcc agagctccgg ggtgctgcgt gtccaggaag ggtacctgtt gagacacatg |
| 24541 | atggtctggg tgcaagttga ccagatgttc ccatcacaat tcataggagg gggtgcccgg |
| 24601 | gaggcagcta ctgacactgc agaccccctt cctgctcagc ccctgcaatt gctcactcta |
| 24661 | gaacaggtca ctccacctga agccagttag ttagccaagc aattgattca cattacgttt |
| 24721 | atataaagca ttacatttta tacgtgatgg gatggggtta gcccatcctc acactttgag |
| 24781 | gtctgtttgt cgttcttttg aactcttata agataaaaaa gtcaatttgt gtaaggacac |
| 24841 | ttgcttgatt tttacagtat taaaaatgtt aagttctttt ttgttgttgt tgagacggag |
| 24901 | tcttgctctg tcaccaaggc tagagtgcag tggcacgatc tcagctcact gcaacctctg |
| 24961 | cctctggttc aagcgattct ccttcctcag cctcctaagt agctgggact acaggtgcat |
| 25021 | gccaccacac ccggctgatt tttgtatttt tagtagagac ggggtttcac catgttggcc |
| 25081 | aggctggctg gtctcgaact cctaacctcg agcgatctgt ctgcctcggc ctcccaaagt |
| 25141 | gctgggatta taggcctgag ccacgcgctc agcaaaaaca ctaaattcta aaagttctaa |
| 25201 | taattaagac aaccgtatat tatatgtgat ttattaagtt tacccctttt gagcagtttt |
| 25261 | gctttcccct tttctgcttg ttttgcagag ttgtgttgtt taggtgaagg tattgagcag |
| 25321 | atccgcacat gctctgtgcc cctccctgct aggatggggc tcgcagtgag aatgtggtgg |
| 25381 | ggacttagat taggaggtct ttggcttgat tgtaaggagg tggaggtaaa ccccaggtag |
| 25441 | agccaaggag accctttgca ggggaatgtc agcagctgct gcagtccctg ctttgggccc |
| 25501 | tcagctcccc tgggagcatg gcatgaggca aactgaagaa cgtcagggga tgtaggaacc |
| 25561 | catgtcgtgg ctgttgctct attacatgga taatcctccc ttctccacgt gaccgatttc |
| 25621 | ctctgagagg cctccctttt aaatgggaac tttgaaatag attctttttg ttttcatgtt |
| 25681 | aaaatgtatt tatttattta aatgacaaaa attatattta tcatgtataa tacaatgttt |
| 25741 | tgaaatatgt atatactgtg gaatggctat attgagctaa ttaacatatg catcacctca |
| 25801 | catacttatt tttgtggtaa gaacacttaa aatctattct cttagtgatt ctcaggaata |
| 25861 | caatatgttg ttattaacta cagtccccat gttgtgcatt agatctctta aatagatatc |
| 25921 | tcttccttct gtctaactga aactttgtat cttttgacca ccatctcctc catctcccca |
| 25981 | gccacacccc gcctccactc cagccctagg taacaactat tctactctct acttctatga |
| 26041 | gttcaacttt cagattccac atataagtga gataatgcag tatttatctt tctgaatctg |
| 26101 | gcttactttg cttaacataa tgtctaaagt agattctttt ttattttatt ttattttttt |
| 26161 | attttttatt ttttaatttt attattatta tactttaagt tttagggtac atgtgcacaa |
| 26221 | tgtgcaggtt agttacatat gtatacatgt gccatgctgg tgtgctgcac ccattaactc |
| 26281 | gtcatttagc attaggtata tctcctaaag ctatccctcc ccactgcccc caccccacaa |
| 26341 | cagtccccag agtgtgatgt tccccttcct gtgtccatgt gttctcattg ttcaattccc |
| 26401 | acctatgagt gagaatatgc ggtgtttggt tttttgttct tgtgatagtt tagtgagaat |
| 26461 | gatttccaat ttcatccatg tccctacaaa ggacatgaac tcatcatttt ttatggctgc |
| 26521 | atagtattcc atggtgtata tgtgccacat tttcttaatc cagtctatca ttgttggaca |
| 26581 | tttgggttgg ttccaagtct ttgctattgt gaatagtgcc gcaataaaca tacgtgtgca |
| 26641 | tgtgtcttta tagcagcatg atttatagtc ctttgggtat atacccagta atgggatggc |
| 26701 | tgggtcaaat ggtatttcta gttctagatc cctgaggaat cgccacactg acttccacaa |
| 26761 | ggtttgaact agtttacagt cccaccaaca gtgtaaaagt gttcctattt ctccacatcc |
| 26821 | tctccagcac ctgttgtttc ctgacttttt aatgattgcc attctaactg gtgtgagatg |
| 26881 | atatctcatt gtggttttga tttgcatttc tctgatggcc agtgatggtg agcatttttt |
| 26941 | catgtgtttt ttggctgcat aaatgtcttc ttttgagaag tatctgttca tgtcctttgc |
| 27001 | ccactttttg atggggttgt ttgttttgtt cttgtaaatt tgtttgagtt cattgtagat |
| 27061 | tctggatatt agccctttgt cagatgagta ggttgcgaaa attttctccc attttgtagg |
| 27121 | attgcctgtt cctccgatga tagtttcttt tgctgtgcag aagctcttta gtttaattag |
| 27181 | atcccatttg tcaattttgg cttttgttgc cattgctttt ggtgttttag acatgaagtc |
| 27241 | cttgcccatg cctatgtcct gaatggtaat tcctaggttt tcttctaggg tttttatggt |
| 27301 | ttatgtctaa catttaagtc tttaatctat cttgaattaa tttttgtata aggtgttagg |
| 27361 | aagggatcca gtttcagctt tctacatatg gctagccagt tttcccagca ccatttatta |
| 27421 | aatagggaat cctttcccca ctggttgttt ttctcaggtt tgtcaaagat cagatagttg |
| 27481 | tagatatgtg gcgttatttc tgagggctct gttctgttcc attgatctat atctctgttt |
| 27541 | tggtactagt actaaagtag attcttttac taaaaaggca gaaggccttg cttttagaca |
| 27601 | gcttggctaa ggattcttga atggctcaaa agtggcaaaa taaaagataa ctcaaataat |
| 27661 | tcagacactc ttaaagccat tcttctgtgt ctggatcgtg gtgtgatgaa tataacataa |
| 27721 | ctttcaggca aatgggtcgt gaggtggtga ttgggccaaa tggatttagg tcagtcctgg |
| 27781 | aaagaacaga gcctgagtcc cctgtctacc caggagctcc tgaggctacc tcctcctcct |
| 27841 | tcagcgagtt tgggtttagg gcaggggtgt ggctttgccg aatatggacc tgagttcaaa |
| 27901 | tatttgctcc accactcatt agctttgcta gtgctgtgac ctgaggcagg tcgcttggcc |
| 27961 | tccttgggcc tcctcagttt cttcctttga aaaatgagga tgataatgtg caccttgtag |
| 28021 | gactattgcg aagcttaagt gagataagtg cctgacaagt agtaggtgct caataaatgg |
| 28081 | gactgtcatt aggtaacata agcatcactt ggggaagcta ctctgcctgt gctgtcaagt |
| 28141 | aagagattcc cttccatgct gccccctcca cgcatttcaa agctgaatag ctcagatttg |
| 28201 | gggcaggcag ctattttgga tataagatta gtttgagaag acttcctgga ggaggggagt |
| 28261 | ttgaggtcaa gtaaggaaaa tgggagagat ggcagtctca gcagggttga gctgagtggc |
| 28321 | ttagacccct gtctgtcctt atccctagga ctgcgcccgg ggcacggcca actgtgtggt |
| 28381 | gttcagctgc ccactctaca gctttgaccg cgcggctgtg ctgcatgtct ggggccgtct |
| 28441 | ctggaacagc acctttctgg aggtgaggat accactcggc tctagtctgt gtttcttctt |
| 28501 | cctccaaagc ccctccagga ctcatttctc ctcctccttc ctcactgtcc aaatgcagca |
| 28561 | tcccccccct tttttttttt tttgagacgg agtcttcctt tgtcccccag gctggagtgc |
| 28621 | agtggtgcga tctcagctca ctgcaacctc tgcctcccag gttcaagagg ttctcctgcc |
| 28681 | tcagcctcct gagtagctga gattacaggc acgtgccacc acacccgata tttttgtatt |
| 28741 | tttcatagag acggggtttt gccatgttgg ccaggctgat ctcaaactcc tgacctcagg |
| 28801 | tgatccaccc tccttggcct cccaaagtgc tgggattaca ggtgtgagcc actgtgccca |
| 28861 | gcccaaacgc agcatactct taacccaaac caggcagaaa catcctctgt gttgggcacc |
| 28921 | attacattta taatttcagg aggtaagggg aagggaggga gacatggggc atttaggaga |
| 28981 | agggtctcct tccctgttcc cttaggagta ctcagctgtg aagtccctgg aagtgattgt |
| 29041 | ccgggccaac atcacagtga agtcctccat aaagaacttg atgctccgag atgcctccac |
| 29101 | agtggtgagc tgcagggttg ggggataggt ggggaggact ctcttctttc ctgctccctc |
| 29161 | cagttcttgt tccattgacc tcttgctcca ttgacccctt gctccccaga tcccagtgat |
| 29221 | ggtatacttg gaccccatgg ctgtggtggc agaaggagtg ccctggtggg tcatcctcct |
| 29281 | ggctgtactg gctgggctgc tggtgctagc actgctggtg ctgctcctgt ggaaggtgag |
| 29341 | gcttggaggt ggggctgatg ggggtggact tcctggttgg gtgagcagca ttttgtattg |
| 29401 | tggttctgtc acccacccac acactcatgt attcagcaaa tctgcactga acacttgccc |
| 29461 | tgcatgtctc aggaactcta ttagtccctg gatgtttcaa agataaatca gatgcaggct |
| 29521 | gggtgcagtg gctcatgcct ggaatctcag cactttggga ggcctaggca ggtggatcac |
| 29581 | ttgaggtcag gagttcgaga ccagcctgga caacatggtg aaaccccatc tctacttaaa |
| 29641 | atacaaaatt agctgggcgt ggtggcacgc acctgtagat ccagctactt gggaggctga |
| 29701 | ggcaggagaa tcgattgaac tcgaaaggtg gggttgcagt gagccgagat ggcgccattg |
| 29761 | cactccagcc tgggtgacag agcgagactc catctcaaaa aaaaaaaaaa aaaaaaaagc |
| 29821 | cgggcatggt ggctcatgcc tgtaatccca gcactttggg aggctgaggt gagtggatca |
| 29881 | cctgaggtca ggagttcaag accaagctgg ccaacatggt gaaatcctgt ctctgttaaa |
| 29941 | aatacaaaca aataaaaaaa attagctggg catggtgggg ggtgcctgta atcccagcta |
| 30001 | cttgggaggc tgagacagga gaatcgcttg aacccgggag gtggaggttg cagtgagcca |
| 30061 | agattgcgcc actgcactgc aacctggggg actgagcagg actctgtctc aaaaaaaaaa |
| 30121 | aaaaaaaaaa aaaaaagaaa tcagatgcag atctcgttct cagggatttt atagtccagt |
| 30181 | gggatgggag tggtgagcct gtagtcaagg acaaggcagg aagtgttgag ttgagaaaat |
| 30241 | ggagtgggag tttagaggga aaaaagcttg agaggaggta gcactggaac cagtcctcag |
| 30301 | aggaaggtgg ggtagtaggg tgaggatgta aggggatgag gtaggtgggg tccagcagga |
| 30361 | ccatggtagg caaagtcgtg gaggcagaga agtgtgatct gacttaacta gaatgtggca |
| 30421 | gtgaaaactg gaaaggtaaa tgtggccaag aaggccaaca agcacggaga acggcctcca |
| 30481 | ggggactgcc ctcatggcgg aactcagagc ttccacatac caggctctgc atggaagcat |
| 30541 | tcttgtacac ccagagctgt ataccgaatc atactggtat ccagggctca atgtaaaagt |
| 30601 | atgcacatgc acacgtcagt acacggaatc acatacatca ccggggtcat atgctgagcc |
| 30661 | atctgccaca tcgggggcta tgttcagaac cacacatgtc cttggggctc tggagaactg |
| 30721 | cccctgcctg tgcctgctct ttgggaccaa tcagagactg agccaccctt tggcaagtgt |
| 30781 | gaatcatcag tagaccaggc tctggagtgg catccaagct gttgggaacc ttcccagtct |
| 30841 | taggggagat tgatgcctct gtgaaactgg tcccaggatg cttgctccct gctcccatgg |
| 30901 | aaggaagaca gagcagagat aggggatcct gtccttcctc aatctttcca tcctctgctt |
| 30961 | cctctgccct gcttctccct cgacatccta gtgtggcttc ttccatcgga gcagccagag |
| 31021 | ctcatctttt cccaccaact atcaccgggc ctgtctggct gtgcagcctt cagccatgga |
| 31081 | agttgggggt ccagggactg tggggtaact gttgtgtgtg tgcatgtgtc tagtgtgtgt |
| 31141 | gggtatgtat gtgtgtgaat ataaggagaa ttcccagcat tcaggggatc aggaatgttc |
| 31201 | agggtgaaaa atgagattgc atgagagtga tgtgaggtga gaggtaatta gaagggtgca |
| 31261 | gatataaggg taagtctaca cagcctgagg cgtgcacaaa gggttggtgg gctcatgggg |
| 31321 | tgtgagcatg tgggagtgtg gggaggtcgc atgcatgctt ttttctatgg gcatgagtgt |
| 31381 | gcaaggagat gaatgtgtaa ggagatgcca tgcagtctga gcatgtagga gtcctgggcc |
| 31441 | tctgagtgtg agtgtgcagg ggcacattcc caccctggtg tgtaagggcc agcatggtcc |
| 31501 | tcatttaggg tggggctaga ctactgtatt gatggacaag agggtattgc tgtaaggggc |
| 31561 | gtgccgccca aggtacaagt acacggggtg catgtttatg aggcttgaga ggtatcattc |
| 31621 | cagagatgca ggggactagg aagccatttt cttcctcagg tcttgtttgt gttctggggg |
| 31681 | acactattgg gtctcttaag aagctagagg gggtacaaat cttgagtccg gtcctgatgc |
| 31741 | tccaccctca aggccctctt tcaggggcca agggagatac ccttggtcct gcctcttgga |
| 31801 | cgcccttctg gtctctctct tgctgtgaat gagaacctgg ggacacagca ctgatgggag |
| 31861 | caccgtgggt gctgcccacc acaccagatg ctattcagcc tatttccttt ctgccgcagc |
| 31921 | agggatggtg agggtgagga gctgtgccgc gctaggcatc tgctccaggg accgcagctc |
| 31981 | ttcaggctcc tcatggtctg gcctggtgtc cttatagatg ggattcttca aacgggcgaa |
| 32041 | gcaccccgag gccaccgtgc cccagtacca tgcggtgaag attcctcggg aagaccgaca |
| 32101 | gcagttcaag gaggagaaga cgggcaccat cctgaggaac aactggggca gcccccggcg |
| 32161 | ggagggcccg gatgcacacc ccatcctggc tgctgacggg catcccgagc tgggccccga |
| 32221 | tgggcatcca gggccaggca ccgcctaggt tcccatgtcc cagcctggcc tgtggctgcc |
| 32281 | ctccatccct tccccagaga tggctccttg ggatgaagag ggtagagtgg gctgctggtg |
| 32341 | tcgcatcaag atttggcagg atcggcttcc tcaggggcac agacctctcc cacccacaag |
| 32401 | aactcctccc acccaacttc cccttagagt gctgtgagat gagagtgggt aaatcaggga |
| 32461 | cagggccatg gggtagggtg agaagggcag gggtgtcctg atgcaaaggt ggggagaagg |
| 32521 | gatcctaatc ccttcctctc ccattcaccc tgtgtaacag gaccccaagg acctgcctcc |
| 32581 | ccggaagtgc cttaacctag agggtcgggg aggaggttgt gtcactgact caggctgctc |
| 32641 | cttctctagt ttcccctctc atctgacctt agtttgctgc catcagtcta gtggtttcgt |
| 32701 | ggtttcgtct atttattaaa aaatatttga gaacaaaacc tctgcctctt tgagtcttgc |
| 32761 | tctggcatcc ccagcatctc tgattctccc tggtgccccc agctcaggaa gaaggtggta |
| 32821 | gtggggagag agggtcaggg gggcttggca gggatgcagg caccatgact tttgtgacca |
| 32881 | gttcctagag acgcatgggt gtagcctcag gaggaaagcg agaggagctt taccatggga |
| 32941 | acgaaggaaa gggacaacat tgggaggcaa acgttgggag actagtccag aaacttgcag |
| 33001 | ttgaggatac aacagggtcc tagtgtttcc tccctgggtc agcgtagaag tagagctttt |
| 33061 | tcactgtcga aggcgggatt gaagttagag attgccctgg cagaggttgg ggagagaggg |
| 33121 | actagtaggt ggtacatgaa tgtcttctct cattctgcta ggtccctctc agtggaagat |
| 33181 | agataggctg gtgggtggct tgttctaatt ggaggctggg gaaggagcaa atgagtgcct |
| 33241 | tggagcttgg gaaagattat ttgacagcct ttcccatgat gtggggccca acaggtagcc |
| 33301 | acttcaaggg agggggctgt cgttccattt ggatttcgga gaactgggcg ttctcaagat |
| 33361 | ccttccaggt ctctctggtg aggcagcagc catccccaat gtgtttccag gtgggctcga |
| 33421 | aaacttgctg ccacatgaag gcccagcttc catatggttc tgccacatgc tcccagaaaa |
| 33481 | agagcacacc tccctgggag ggatgagaga gttagtgtca cttggctcca ttctgaccct |
| 33541 | gctccctatc gcgctgatca agacagtcaa acttcttggt gaaatgggag ctgctggtct |
| 33601 | gagagttcat ggagcaaact gtcgtgcccc tgaccttttc tgcatcccct gctaccgcct |
| 33661 | tcaaagtgca tgtgaagaac ctcataactg aaaggagtgg ctggtgggag atctttaaag |
| 33721 | cctgaccaga ataaccgagg gcatgcagaa ggggaaactg agaaacagga gtggagggct |
| 33781 | cattgtatta ttattattat tattattttt tgagacagag tctcagtctg ttgctcaggc |
| 33841 | tggagtgcaa tggcgtgatc tcggcgcact gcaacctcca cctctcaggt tcaagcgatt |
| 33901 | ctcctgcctc agcctcccaa gtagctggga ttacaggcat gagccaccac gcccggctac |
| 33961 | tttttgtatt tttcagtaga gacagggttt caccatgttg gccagtctgg tttcaaactc |
| 34021 | ctggcctcaa gagatccacc tgccttgcct cccaaagtgc tggattacag gcgtgagcca |
| 34081 | cctcgcccag ctgggcttat tgtattctga gatacacgca caacctctct cacaaaaccc |
| 34141 | agatgcatgc acaagctgct tcctggacat cgctgtgaac atcccaggga aacctcaggc |
| 34201 | ccaactcttg actgaagctg gggttcctca agctcctcgc ctcactatac tctccctagg |
| 34261 | tgatctcaaa cctagtaagt ccaaaacagc tttcttccat ctcagcaaat ggcacttttc |
| 34321 | atcagctctc ccctcagttg ctcatgctag aaacctggaa actattctga acgttctctc |
| 34381 | cacagtctga tggattctac tctacttccc ctactatctt tacctagaac actcttttat |
| 34441 | tttccccacc ccgtccacac tctgcttaat ccctcagatc tctctctctc tctctctttt |
| 34501 | tatttattta tttatttttt ttgatacagg gtctcactct gtggcccagg ctggagtgca |
| 34561 | gtggtgcgat catagttcac tgtaactttg aactcgggct caagtgatcc tcccacctca |
| 34621 | gcttcctgag tagctaggac tataggcatg tgccaccacg cctggctagc ttttgtattt |
| 34681 | tttttttttt ttttttttgt agagacggca tctcactatg ttgccttggc tggtcttg |

Other ITGA7 sequences include:

(SEQ ID NO: 35)
ACCAATACCCTGACCTGCTG,
(SEQ ID NO: 36)
CTATAGCTGCTGGGGACTGC,
ITA7(h)
(SEQ ID NO: 37)
5′-ACCAATACCCTGACCTGCTG
(SEQ ID NO: 38)
5-CTATAGCTGCTGGGGACTGC

Exemplary laminin alpha 4 sequences include:

| (SEQ ID NO: 39) | |
|---|---|
| 1 | MALSSAWRSV LPLWLLWSAA CSRAASGDDN AFPFDIEGSS AVGRQDPPET SEPRVALGRL |
| 61 | PPAAEKCNAG FFHTLSGECV PCDCNGNSNE CLDGSGYCVH CQRNTTGEHC EKCLDGYIGD |
| 121 | SIRGAPQFCQ PCPCPLPHLA NFAESCYRKN GAVRCICNEN YAGPNCERCA PGYYGNPLLI |
| 181 | GSTCKKCDCS GNSDPNLIFE DCDEVTGQCR NCLRNTTGFK CERCAPGYYG DARIAKNCAV |
| 241 | CNCGGGPCDS VTGECLEEGF EPPTGCDKCV WDLTDDIRLA ALSIEEGKSG VLSVSSGAAA |
| 301 | HRHVNEINAT IYLLKTKLSE RENQYALRKI QINNAENTMK SLLSDVEELV EKENQASRKG |
| 361 | QLVQKESMDT INHASQLVEQ AHDMRDKIQE INNKMLYYGE EHELSPKEIS EKLVLAQKML |
| 421 | EEIRSRQPFF TQRELVDEEA DEAYELLSQA ESWQRLHNET RTLFPVVLEQ LDDYNAKLSD |
| 481 | LQEALDQALN HVRDAEDMNR ATAARQRDHE KQQERVREQM EVVNMSLSTS ADSLTTPRLT |
| 541 | LSELDDIIKN ASGIYAEIDG AKSELQVKLS NLSNLSHDLV QEAIDHAQDL QQEANELSRK |
| 601 | LHSSDMNGLV QKALDASNVY ENIVNYVSEA NETAEFALNT TDRIYDAVSG IDTQIIYHKD |
| 661 | ESENLINQAR ELQAKAESSS DEAVADTSRR VGGALARKSA LKTRLSDAVK QLQAAERGDA |
| 721 | QQRLGQSRLI TEEANRTTME VQQATAPMAN NLTNWSQNLQ HEDSSAYNTA VNSARDAVRN |
| 781 | LTEVVPQLLD QLRTVEQKRP ASNVSASIQR IRELIAQTRS VASKIQVSMM FDGQSAVEVH |
| 841 | SRTSMDDLKA FTSLSLYMKP PVKRPELTET ADQFILYLGS KNAKKEYMGL AIKNDNLVYV |
| 901 | YNLGTKDVEI PLDSKPVSSW PAYFSIVKIE RVGKHGKVEL TVPSLSSTAE EKFIKKGEFS |
| 961 | GDDSLLDLDP EDTVFYVGGV PSNFKLPTSL NLPGFVGCLE LATLNNDVIS LYNFKHIYNM |
| 1021 | DPSTSVPCAR DKLAFTQSRA ASYFFDGSGY AVVRDITRRG KFGQVTREDI EVRTPADNGL |
| 1081 | ILLMVNGSMF FRLEMRNGYL HVFYDFGFSS GRVHLEDTLK KAQINDAKYH EISIIYHNDK |
| 1141 | KMILVVDRRH VKSMDNEKMK IPFTDIYIGG APPEILQSRA LRAHLPLDIN FRGCMKGFQF |
| 1201 | QKKDFNLLEQ TETLGVGYGC PEDSLISRRA YFNGQSFIAS IQKISFFDGE EGGFNFRTLQ |
| 1261 | PNGLLFYYAS GSDVESISLD NGTVIMDVKG IKVQSVDKQY NDGLSHEVIS SVSPTRYELI |
| 1321 | VDKSRVGSKN PTKGKIEQTQ ASEKKFYFGG SPISAQYANF TGCISNAYFT RVDRDVEVED |
| 1381 | FQRYTEKVHT SLYECPIESS PLFLLHKKGK NLSKPKASQN KKGGKSKDAP SWDPVALKLP |
| 1441 | ERNTPRNSHC HLSNSPRAIE HAYQYGGTAN SRQEFEHLKG DFGAKSQFSI RLRTRSSHGM |
| 1501 | IFYVSDQEEN DEMTLFLAHG RLVYMENVGH KKLKIRSQEK YNDGLWHDVI FIRERSSGRL |
| 1561 | VIDGLRVLEE SLPPTEATWK IKGPIYLGGV APGKAVKNVQ INSIYSESGC LSNLQLNGAS |
| 1621 | ITSASQTFSV TPCFEGPMET GTYFSTEGGY VVLDESENIG LKFEIAFEVR PRSSSGTLVH |
| 1681 | GHSVNGEYLN VHMKNGQVIV KVNNGIRDES TSVTPKQSLC DGRWHRITVI RDSNVVQLDV |
| 1741 | DSEVNHVVGP LNPKPIDHRE PVFVGGVPES LLTPRLAPSK PFTGCIRHEV IDGHPVSFSK |
| 1801 | AALVSGAVSI NSCPAA |
| (SEQ ID NO: 40) | |
| MAAEDELQLPRLPELFETGRQLLDEVEVATEPAGSRIVQEKVEK | |
| GLDLLEKAAEMLSQLDLESRNEDLEEIASTDLKYLLVPAFQGALTMKQVNPSKRLDHL | |
| QRAREHFINYLTQCHCYHVAEFELPKTMNNSAENHTANSSMAYPSLVAMASQRQAKIQ | |
| RYKQKKELEHRLSAMKSAVESGQADDERVREYYLLHLQRWIDISLEEIESIDQEIKIL | |
| RERDSSREASTSNSSRQERPPVKPFILTRNMAQAKVFGAGYPSLPTMTVSDWYEQHRK | |
| YGALPDQGIAKAAPEEFRKAAQQQEEQEEKEEEDDEQTLHRAREWDDWKDTHPRGYGN | |
| RQNMG | |
| (SEQ ID NO: 41) | |
| 1 | gcacttcgcg ctcaagcgac cggatcttca aaccgtggga gtggtgcggc ggctagagtc |
| 61 | cctggactcc tcaacctagg gagctactcg cgagatgcct acgacttgta acgggctgcc |
| 121 | tggtaaaatg agtctatgga aacggttgcc agggccggct aacagcggct cccggaagtc |
| 181 | ctttgatgct ttgttaacag tgaagctact ggaccaatga ggtgcttctt ccggttttgt |
| 241 | ccgcgctcgc ctaattcttc tttatcaagg ttgcctttga ccccggaaaa gagatcttcc |
| 301 | gggttcctct ctccccaaga tggctgctga ggacgagtta cagctgccgc ggctccccga |
| 361 | gctgttcgaa actggtagac agttactgga cgaagtagaa gtggcgactg aacccgccgg |
| 421 | ttcccggata gtccaggaga aggtgttcaa gggcttggac ctccttgaga aggctgccga |
| 481 | aatgttatcg cagctcgact tgttcagccg aaatgaagat ttggaagaga ttgcttccac |
| 541 | cgacctgaag taccttttgg tgccagcgtt tcaaggagcc ctcaccatga aacaagtcaa |
| 601 | ccccagcaag cgtctagatc atttgcagcg ggctcgagaa cactttataa actacttaac |
| 661 | tcagtgccat tgctatcatg tggcagagtt tgagctgccc aaaaccatga acaactctgc |
| 721 | tgaaaatcac actgccaatt cctccatggc ttatcctagt ctcgttgcta tggcatctca |
| 781 | aagacaggct aaaatacaga gatacaagca gaagaaggag ttggagcata ggttgtctgc |
| 841 | aatgaaatct gctgtggaaa gtggtcaagc agatgatgag cgtgttcgtg aatattatct |
| 901 | tcttcacctt cagaggtgga ttgatatcag cttagaagag attgagagca ttgaccagga |
| 961 | aataaagatc ctgagagaaa gagactcttc aagagaggca tcaacttcta actcatctcg |
| 1021 | ccaggagagg cctccagtga aacccttcat tctcactcgg aacatggctc aagccaaagt |
| 1081 | atttggagct ggttatccaa gtctgccaac tatgacggtg agtgactggt atgagcaaca |
| 1141 | tcggaaatat ggagcattac cggatcaggg aatagccaag gcagcaccag aggaattcag |
| 1201 | aaaagcagct cagcaacagg aagaacaaga agaaaaggag gaagaggatg atgaacaaac |
| 1261 | actccacaga gcccgggagt gggatgactg gaaggacacc catcctaggg gctatgggaa |
| 1321 | ccgacagaac atgggctgat cttcccacaa caccacagga ctgcagggtg cacaactccc |
| 1381 | ctgccaagga aaaccatgca gtcctcccct ccctggtctc ctgcttcagc tctgtacaac |
| 1441 | gagggcaaag atgctaaatc ttgctttgca ttcagtaaag tgtcaagtga ttaagtgtgt |
| 1501 | atttgtaccc tagatgatat gaaccagcag tcttgttttg gcatcatcct catcatgttg |
| 1561 | tattccagct tcttaagtgg aaggaaaaga gtgctgagaa atggctctgt ataatctatg |
| 1621 | gctatccgaa ttctctgaaa aaataataaa agtcccctct attatatgag cctgtacaga |
| 1681 | aa |

### Example 3

Local administration of lipid-silica nanohybrid-carried forskolin modulates thermogenesis in human adipocytes and impedes weight gain in mice.

### Introduction

Obesity is characterized by an increase in adipose mass and predisposes individuals to cardiovascular disease, type 2 diabetes, hypertension, stroke, and many cancers such as endometrial, liver and pancreatic cancer among others.(1) There are currently seven FDA-approved drugs for obesity that function as glucagon-like peptide-1 (GLP-1) agonist (Saxenda, Wegovy), melanocortin receptor agonist (Imcivree) or lipase inhibitor (Xenical) that mainly work on curbing hunger and can be administered orally or subcutaneously without directly targeting the fat-storing adipose tissue. Despite the success of these drugs in weight gain prevention or weight loss, they still present drawbacks mainly associated with a quick weight gain upon medicine halt, not to mention that some of these drugs are showing serious side effects such as the 4-to-9 times increased risk of stomach paralysis, pancreatitis and bowel obstruction when GLP-1 agonists are administered (2.) Considering that research to lessen adiposity is skyrocketing along with the drug market, the development of local or adipose-targeted therapeutic approaches that lessen or eliminate obesity-associated morbidity would have a transformative impact on the population's health and healthcare spending.

The distribution of adipose tissue in the human body is heterogeneous and is associated with metabolic disease risk (3, 4). White adipose tissue (WAT) contains adipocytes that are larger in size, have a single lipid droplet, and are primarily involved in energy storage while brown adipose tissue (BAT) adipocytes are smaller in size, contain multiple lipid droplets, and their primary role is thermogenesis through lipid oxidation and uncoupled respiration (5). A third adipocyte subtype, the beige/brite adipocyte (bAT), arises predominantly in white depots in response to cold, and its morphology is intermediate between white and brown adipocytes in both cell size and mixed content of large and small lipid droplets (6). Thermogenic adipose tissue has been shown to have an inverse correlation with adiposity, indicating that it might be possible to increase whole-body energy expenditure by increasing the number and/or activity of thermogenic adipose tissues (BAT and bAT) (7).

The sympathetic nervous system (SNS) and β-adrenergic signaling roles in stimulating adipose tissue thermogenesis when exposed to cold has been recognized for a considerable period (8.) When the body encounters cold temperatures, the SNS releases norepinephrine, which then operates through β-adrenergic receptors (β-ARs) to initiate the cyclic adenosine monophosphate (cAMP) and protein kinase A (PKA) signaling pathway (9, 10). The PKA phosphorylates p38 and cAMP response element-binding protein (CREB), all of which promote the transcription of thermogenic genes. Therefore, pharmacological activators of these pathways, including the 1B3-AR agonist CL-316,243 (6, 11), the peroxisome proliferator-activated receptor γ (PPAR γ) agonists rosiglitazone (Rosi) (12) or GW0742 (13), have been employed to promote adipocyte browning as an alternative to cold temperature. Most of these molecules and other therapeutics of interest will have an improved outcome if they are carried by a vehicle that increases efficacy while reducing off target effects. Harnessing the advantages of nanoparticles to deliver agents that impact white adipocyte's metabolic function is an emerging topic. However, despite the predominant presence of mature adipocytes in the adipose tissue (14), the handful of studies in this area (using dibenzazepine, resveratrol, emodin, Rosi (15-19)) have only focused on murine adipose stem cells.

On the other hand, the adenylyl cyclase activator forskolin (FSK), a naturally extracted diterpenoid from the tropical plant Coleus forskohlii has shown encouraging (6, 20, 21) but sometimes conflicting results in modest size clinical trials to treat heart disease and induce weight loss in obese men (22). More recently, intraperitoneally injected FSK demonstrated improvement in glucose metabolism and adipocytes size in high fat diet-fed mice (7). Like most water-insoluble therapeutics, the efficient delivery of FSK into white adipocytes is compromised by its stability and low bioavailability in its free or unprotected form within the blood plasma (23, 24) in addition to off-target non-specificity, and that might explain the modest and conflicting results obtained upon free FSK administration. Therefore, a robust and biocompatible delivery vehicle with homogenous, reproducible, and scalable features is required to emphasize the role of FSK in obesity treatment. As lipid-coated mesoporous silica nanoparticles (LCMSNs) have proven to be an outstanding nanoplatform in molecular delivery to cancer and immune cells (25-27), provided herein their unique properties were leveraged to deliver the FSK thermogenic agent for the first time into human differentiated adipocytes. The following sections highlight the strategy to implement a nanosystem that is retained in adipose tissue and efficiently deliver FSK to modulate thermogenesis in human adipocytes and help in weight gain prevention in high fat diet mice.

### Methods

### 1. Synthesis of FSK Loaded Lipid Coated Mesoporous Silica Nanoparticles (LCMSNs)

### 1.1 MSN Synthesis

Dendritic mesoporous silica nanoparticle of ~100 nm diameter and of 3-4 nm average pore diameter were synthesized (28). Size and zeta potential were measured using a Zetasizer machine (Malvern Instruments. Ltd.) and TEM images of the nanoparticles were also taken. Briefly, in a 100 mL round bottom flask, 0.18 g (1.8 mmol) TEA, 24 mL (72.6 mmol) CTAC, and 36 mL of distilled water were stirred at 400 rpm and heated to 40° C. for an hour. Then 20 mL of a solution of TEOS in cyclohexane (40% v/v) was added to form the biphasic system. The reaction was kept at 40° C. for 16 h. The bottom aqueous phase containing the NPs suspension was centrifuged and the isolated pellet washed in ethanol through successive sonication centrifugation steps. The removal of CTAC was achieved by washing the suspended 1% HCl in ethanol. Fluorescently labeled MSNs were made by adding 2 μL APTES during the synthesis, followed by post-functionalization with the dye of interest, namely Cyanine 3-NHS ester, DyLight 633-NHS ester, or DyLight 633 NHS ester.

### 1.2. FSK-Free and -Loaded Liposome Synthesis

The liposome formation follows previously described report. Liposomal nanoparticles synthesized at 5 mg/mL in PBS are composed of 57 mol % 1,2-dipalmitoyl-sn-glycero-3-phosphocholine (DPPC), 25 mol % 1,2-dimyristoyl-sn-glycero-3-phospho-(1′-rac-glycerol) (DMPG), 10 mol % Cholesterol, and 8 mol % 1,2-distearoyl-sn-glycero-3-phosphoethanolamine-N-[carboxy-(polyethylene glycol)-2000](DSPE-PEG2000), and 1,2-dioleoyl-sn-glycero-3-phosphoethanolamine-N-(lissamine rhodamine B sulfonyl) (ammonium salt) at 0.5% mol when liposomes are made red fluorescent or 1,2-distearoyl-sn-glycero-3-phosphoethanolamine-N-[amino(polyethylene glycol)-2000]-N-(Cyanine 5) when made far-red fluorescent to be used for in vivo biodistribution (Avanti Polar Lipids). Stock lipids were prepared in chloroform (Sigma) solvent and were mixed in a borosilicate glass vial and stored at −25° C. A rotary evaporator was used to evaporate the solvent resulting in lipid films that were hydrated in 1 mL 1×PBS under 20 minutes sonication (Branson) but the sonication time is reduced to 5 minutes in a cold bath when using the cy5 lipid.

To prepare FSK-loaded liposomes (5 mg basis), an ethanolic solution of FSK (10 mg/mL, 10% weight loading FSK m=0.5 mg V=50 μL or 25% FSK m=1.25 mg V=125 μL) is added to the lipid mixture and evaporated altogether in the rotavap for 10 minutes. All other steps remain the same and the final mixture with FSK will be opaquer than the FSK-free liposomes due to the presence of “suspended” FSK in an aqueous medium. Hydrodynamic size and zeta potential were measured via Zetasizer (Malvern Instruments).

### 1.3. LCMSN Synthesis and Forskolin Loading.

1.3.1. FSK loading in MSN: MSNs (1 mg) were centrifuged in a microcentrifuge at 20,000×g for 10 min to remove ethanol and resuspended in FSK solution (DMSO, 10 mg/mL, 5 mg) for 16-20 h under gentle shaking, at room temperature. MSNs were then centrifuged (20 min, 20,000 rcf) and the supernatant was carefully removed to the highest extent possible. Failing to do so will induce FSK precipitation upon water addition (see next step).

1.3.2. FSK-loaded LCMSN: The isolated pellet (1 mg MSN) from the previous step is suspended in 1 mL water (1 mg/mL) before the addition of 1 mL of FSK-loaded liposomes (5 mg/mL) under sonication for 30 seconds. The resulting FSK-loaded LCMSNs were collected by centrifugation at 20,000×g and washed with 1 mL PBS before resuspension in PBS at 1 mg/mL (MSN weight), by pipetting under sonication. The FSK-LCMSN were then tested for hydrodynamic size and dispersity by DLS (Malvern Zetasizer). For in vivo efficacy experiments, the experiment was scaled up 10 times without any noticeable impact to particles quality.

The FSK loading percentage was obtained by employing thermogravimetric analysis and comparing the FSK-LCMSN to MSN and FSK-free LCMSNs. The loading percentages were ~11% and 30% respectively when liposomes are prepared with 10% or 25% FSK.

2. Human Adipose Derived Stem Cells (hASC) Culture and Adipocyte Differentiation.

Commercially available hASCs (PT-5006, Batch 0,000,535,975, Lonza) were used between passage 2-4. Cells were seeded in multi well plates in complete growth media (GM, Dulbecco's modified Eagle's Ham's F12 medium supplemented with 10% fetal bovine serum, and 1% antibiotic-antimycotic). The media was changed every other day. When cells reached confluence, adipocyte differentiation was initiated (designated as day 0). Cells were then maintained in GM supplemented with 850 nM insulin, 250 μM isobutyl-methylxanthine, 125 nM indomethacin, 0.5 μM dexamethasone, 1 μM rosiglitazone and 120 nM triiodothyronine for 14 days. Differentiation media was changed every other day.

### 3. Evaluation of Cytotoxicity in Mature Adipocytes Following LCMSNs Treatment In Vitro

The CytoTox-Glo (Promega, Madison, Wisconsin) cytotoxicity assay was used to assess any LCMSNs toxicity in mature adipocytes. The assay measures dead-cell protease activity using a luminogenic peptide substrate. Briefly, mature adipocytes (10.000 cells) were incubated with different concentrations (20, 50, 100, 200, 500, and 1000 μg/mL) of LCMSNs for 24 h. Following the manufacturer's protocol, luminescence of dead-cell protease activity (related to membrane integrity) was read after LCMSN treatment prior to and following addition of a lysis reagent. The ratio of luminescence of dead cell to total cell luminescence was calculated for each condition to determine the percentage of dead cells after treatment. Untreated adipocytes were used as negative control.

4. Uptake of LCMSNs in Mature Human Adipocytes (Differentiated hASC) In Vitro and in Mice Pre-Isolated Inguinal WAT Ex Vivo

Differentiated human adipocytes and pre-isolated inguinal WAT (iWAT) were incubated for various time points with LCMSN to evaluate uptake and internalization. Uptake of cy3-labeled particles was measured using (4.1) A BioTek Cytation 5 cc and (4.2) Flow cytometry to evaluate the number of adipocytes with LCMSN uptake in vitro, (4.3) a Leica TCS SP8 Confocal Microscope (Leica Microsystems Inc., Deerfield, IL) to assess the uptake of Dy633-LCMSN in pre-isolated inguinal WAT ex vivo.

### 4.1 Microplate Reader:

hASC were plated in a 24-well culture plate at a density of 4×10⁴ cells per well with 500 μL of CM. After reaching confluency, cells were differentiated to adipocytes as previously described. On day 14 of differentiation, medium was exchanged for treatment medium containing LCMSNs at 50 μg/ml. Adipocytes were incubated with LCMSNs for different time intervals (3 h, 6 h, 24 h or 48 h). Following treatment, cells were washed twice with PBS and resuspended in PBS. Nanoparticle fluorescence was measured using a BioTek Cytation 5. Nanoparticle-free adipocytes were used as negative controls for each experimental condition to obtain background fluorescence measurements.

### 4.2 Flow Cytometry:

Mature adipocytes in 6-well plates were treated with rhodamine fluorescently labelled LCMSNs for 3, 6, 24, and 48 h followed by trypsinization to detach cells, centrifugation, and washes with PBS. Flow cytometry (BD Accuri C6 Plus) was utilized to determine the uptake efficiency of adipocytes with LCMSNs. A fluorescence threshold was set utilizing non-treated adipocytes. Measurement parameters were set the same for all samples, and each run was set to count 10⁴ events. The total percentage of each cell layer containing the fluorescent labeled LCMSNs was determined from the area corresponding to higher intensities than the threshold.

### 4.3 Confocal Imaging:

For in vitro imaging, hASC were plated on 4-chamber Labtek slides (Thermo Scientific) at a density of 7×10⁴ cells per well, differentiated for 14 days, treated with LCMSNs for different time intervals (3-24h), washed with PBS, fixed in 4% paraformaldehyde and stained with BODIPY (1:100).

For ex vivo imaging, the analysis of LCMSN uptake in adipose tissue ex vivo, inguinal WAT was dissected from adult mice, placed in 6 wells plates, and incubated at 37° C. (5% 02, 95% CO2) for 24 h in DMEM-F12 (Sigma-Aldrich, D6421) containing DyLight 633 labeled LCMSNs. Following sequential rinses in 0.1 M phosphate-buffered saline, preparations were fixed using 4% paraformaldehyde for 3 h at room temperature and permeabilized using 0.5% Triton X-100 (Sigma-Aldrich) in 1M Tris-buffered saline. Preparations were then counterstained using DAPI to stain nuclei and Griffonia simplicifolia isolectin conjugated with Rhodamine to label endothelial cells. Preparations were stored in TBS until imaging. A confocal laser-scanning microscope (Leica TCS SP8 Confocal Microscope; Buffalo Grove. IL) was used to image adipocytes with LCMSNs. In vitro, adipocytes labeled by BODIPY and Rhodamine labeled LCMSNs were imaged simultaneously in two channels. For ex vivo imaging, adipocytes labeled by BODIPY and Dy633-LCMSNs were imaged simultaneously in two channels, with rhodamine-labeled endothelial tissue, and DAPI nuclei imaged in series. Confocal image stacks were obtained with a 63×oil immersion lens (NA 1.4). Laser intensity, confocal aperture, and photomultiplier gain were kept constant across samples. Confocal image stacks were processed and analyzed using Leica LASX software version 3.5.2 (Wetzlar, Germany). A region of interest (ROI) outlining an adipocyte was drawn from the maximum intensity projection. Within this ROI, the LCMSN fluorescence intensity was measured.

5. Biodistribution of LCMSNs after Subcutaneous Injection in Mice.

Male C57BL/6 J mice (8- to 12-week-old, Jackson Laboratory) were injected subcutaneously close to the inguinal WAT (iWAT) with either 1) 200 μg of FSK-loaded Dy633-fluorescently labeled LCMSNs at 1 mg/mL, 2) 200 μg of unloaded Dy633-fluorescently labeled LCMSNs, or 3) unlabeled 20 μg free FSK at 2.5 mM concentration (same amount loaded in the LCMSN). All treatments were dissolved into saline. Non-invasive optical imaging system KINO was used after different times of injection (3 h, 6 h, 24 h, and 48 h) to assess overall distribution and retention of the LCMSNs in iWAT. Animals were sacrificed and different tissues (heart, lung, kidney, liver, iWAT, visceral WAT) were isolated and imaged to measured LCMSNs presence. Treated iWAT was cut into 3 pieces to be immediately frozen in liquid nitrogen followed by storage at −80° C. for metabolic assays or stored in Trizol solution or in RIPA buffer for molecular assays. The animal protocol was approved by the animal care and use committee of The University of Texas at San Antonio, San Antonio, TX.

6. Efficacy of LCMSNs Loaded with FSK in Thermogenic Activity of Adipocytes and Adipose Tissue

### 6.1. Thermogenic Biomarkers Detection by Real-Time PCR

RNA from iWAT tissue and cells was isolated and purified using a Qiagen Rneasy Mini Kit (Valencia, CA) according to manufacturer guidelines. mRNA concentrations were measured using a Take3 Micro-Volume Plate (BioTek, Winooski, VT), then normalized to 150 ng of mRNA for conversion to cDNA. cDNA was synthetized using random hexamer primers and an iScript cDNA-synthesis kit (Biorad). The quantitative RT-PCR reactions were performed using the So Advanced™ Universal SYBER Green Supermix kit (Biorad) in a CFX96 Touch Real-Time PCR Detection System (BioRad, Hercules, CA). Fold expression levels were calculated using the 2-AACt method. Transcript levels were normalized to 18S ribosomal RNA levels in all experiments. Primer (Table 1) specificity was tested by assessing the melting curve.

### 6.2. Oxygen Consumption Rate

hASC were seeded at 6,000 cells per well into XF96 V3 PS tissue culture microplates and differentiated into mature adipocytes for 14-days. After which, cells were treated with free FSK, LCMSNs, and LCMSNs-FSK for 3 and 6 hours. Before the assay, cells were placed in a 37° C. incubator without CO2 for 45 min. A Seahorse XFe96 Flux Analyzer (Seahorse Bioscience) was used to analyze the cellular metabolic activity of adipocytes after different treatments. The XF Analyzer measures the oxygen consumption rate (OCR) of live cells in real-time. After basal measurements, oligomycin (1.5 μM) was added to the culture wells (at 16 min) to inhibit ATP synthase, reducing the contribution of ATP production, and revealing proton leak in the electron transport chain (ETC, after correcting for non-mitochondrial oxygen consumption). Maximum respiration was induced by exposure to the ionophore carbonyl cyanide-ptrifluoromethoxyphenylhydrazone (FCCP, 2.0 μM) at 36 min. Finally, rotenone and antimycin A (0.5 μM/0.5 μM) were used at 56 min to inhibit the ETC enzymes upstream of oxygen consumption, thus eliminating all mitochondrial oxygen expenditure. The final OCR represents the background non-mitochondrial oxygen consumption of the cells. Subtracting the final OCR after rotenone/antimycin A treatment from the initial OCR before oligomycin treatment gives the basal mitochondrial metabolism or the mitochondrial OCR at a resting state. At least three measurements were taken prior to, and following, the application of each drug solution. Results were normalized to cellular protein levels which were quantified using a BCA protein assay kit (ThermoFisher), and thus, cellular respiration are given in arbitrary units (AU).

### 6.3. Thermogenic Biomarkers Detection by Western Blot

iWAT and BAT were lysed in RIPA buffer (EMD Millipore, Billerica, MA) with protease inhibitor (Fischer Scientific, Waltham, MA) and homogenized. Samples were then centrifuged (20,000 g for 15 min), and protein was extracted and quantified using the Bio-Rad DC protein assay (Bio-Rad Laboratories, Hercules, CA). 20 μg of protein sample was electrophoretically denatured on 4-12% Bis-Tris Gel (Invitrogen, ThermoFisher Scientific) and transferred to immune-Blot PVDF Membranes (Bio-Rad). After which, membranes were blocked using a 5% non-fat milk solution (Sigma), followed by an overnight incubation with primary antibody for UCP1 (Cell Signaling Technology) and Actin (Cell Signaling Technology) at RT. After washing with TBST solution (0.10% Tween20, 150 mM NaCl, and 20 mM Tris), the membranes were incubated with a secondary anti-rabbit IgG antibodies for 1.5 h at RT. The immunoreactive bands were detected using SuperSignal West Gemto Maximum Sensitivity Substrate kit (Thermo Scientific) and with a FluorChem M System (Bio-Techne).

### 6.4. Evaluation of Glucose Uptake in Mature Adipocytes In Vitro

Differentiated mature adipocytes cultured in a 96-well plate were treated with LCMSNs loaded with FSK, un-loaded LCMSNs, and free FSK for 3 h, 6 h, 24 h, and 48 h. Glucose Uptake-Glo™ Assay (Promega) was used to measure glucose metabolism and the effect of treatments. The experiment was run according to manufacturer's protocol. Briefly, on the day of the assay, cell media was replaced with DMEM containing TX RealTime-Glo assay reagent and 50 μM of cytochalasin B and was then incubated for 30 min at 37° C. After which 10 μM insulin was added, and the samples were further incubated for one hour. Following incubation, 1 mM of 2-deoxyglucose was added to the samples, and after 10 min, 2-deoxyglucose-6-phosphate detection reagent was added and incubated further at room temperature for an hour. Lastly, a microplate reader system (BioTek Synergy 2) was used to record the luminescence signal.

### 6.5. Evaluation of Lipolysis in Mature Human Adipocytes In Vitro and in Mice Tissue In Vivo.

iWAT was isolated from the mice at the end of the timepoint, the measurement of lipolysis was conducted following the protocol provided by MAK195 Lipolysis Kit (Sigma-Aldrich). Briefly, freshly isolated adipose tissue was minced, and 0.2% collagenase was added and incubated in an orbital shaker at 37° C. for 30 min. Collagenase stop buffer was added and samples was filtered through a 100 μm cell strainer. The filtrate was centrifuged at 500×g for 10 min, and the top layer (adipocyte) was transferred into a fresh tube. The adipocyte was washed with Wash Buffer and centrifuged twice. Adipocyte lipolysis buffer was added to the adipocytes which was then stimulated for lipolysis with 100 nM isoproterenol for 3 hours. Colorimetric assay reaction mix was then added to the samples which was then incubated at room temperature for 30 minutes, after which a microplate reader was used to measure the sample absorbance (A570).

For in vitro lipolysis assay, the MAK211 Lipolysis Colorimetric Assay Kit (Sigma-Aldrich) was used to measure the concentration of glycerol generated during the triglyceride lipolysis process. In this context, hASC were grown and differentiated as explained before. Isoproterenol was added to cells to stimulate lipolysis followed by assay reaction reagents. Lipolysis was determined by measuring a colorimetric product (using the microplate reader BioTek Synergy 2) with absorbance at 570 nm (A570) proportional to the amount of glycerol present.

### 7. In Vivo Obesity Prevention

To evaluate the physiological effect of increased thermogenic activity in iWAT with LCMSN-FSK treatment, 12-week-old C57BL/6J mice from Jackson's Laboratory (Bar Harbor, ME) were fed a high fat diet (HFD, 60% energy from fat, D12492, Research Diets, Inc, New Brunswick, NJ) for six weeks. The animals were kept at UTSA Laboratory Animal Resource Center under standard mouse housing conditions and had access to HFD diet and water ad libitum. The animals were separated into four treatment groups: saline, free FSK, FSK loaded LCMSN, or FSK loaded liposomes (n=3) and were injected twice per week subcutaneously in the iWAT depots at 50 μg FSK/mouse. After two weeks of injections, the treatment dose was increased to 500 μg FSK/mouse injected nanoparticles (equivalent to 25 mg/kg and to 0.5× dose of Resveratrol per injection) as no significant body weight differences were observed between the treatment groups. After six weeks, the animals were humanely euthanized with CO2 inhalation. iWAT, vWAT, heart, lung, liver, and kidney were collected. The animal protocol was approved by the animal care and use committee at the University of Texas at San Antonio, TX.

### 8. Statistical Methods

Graphpad Prism Software 9 (GraphPad Software, Inc., La Jolla, CA) was used to run unpaired T tests with Welch's correction and one way analysis of variance (ANOVA) tests with Tukey or Dunnet multiple comparison analysis (as specified in each figure) to determine differences between groups. Statistical significance was defined when p<0.05. All results are presented as mean±standard error of the mean (SEM).

### Results

I. Forskolin-Loaded LCMSN were Successfully Generated and Characterized.

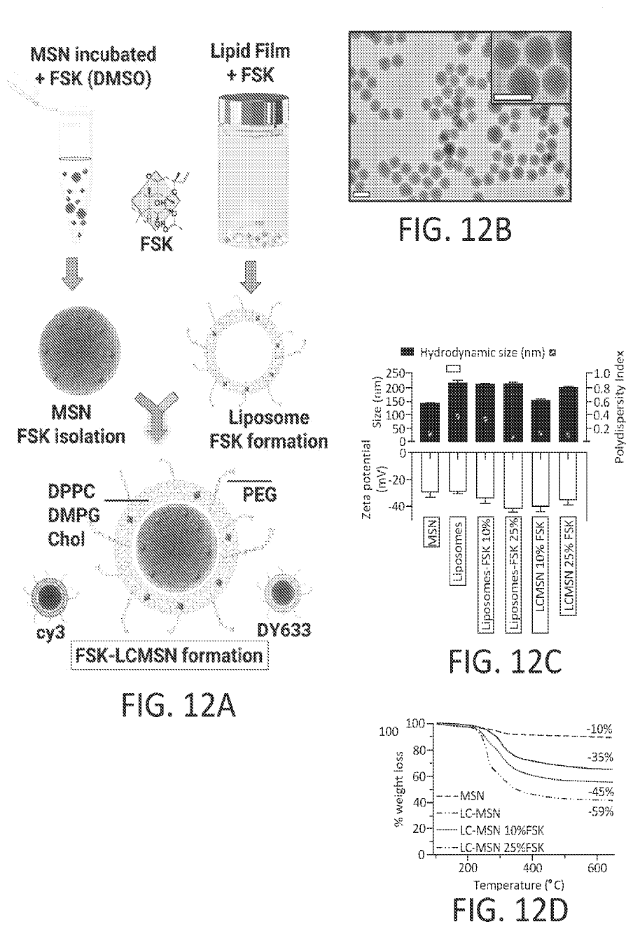

The implementation of FSK-LCMSN followed the schematic in FIG. 12A. Dendritic MSN with a pore size around 3-4 nm were made via the biphasic stratification approach and exhibit a hydrodynamic size of 140 nm and a low polydispersity index PDI (<0.1), comparable to their TEM physical size (FIG. 12B) and a negatively charged surface (ζ=−28 mV) characteristic of a bare silica surface (FIG. 12C). A previous report has shown a reverse correlation between dense nanosilica size (2 sizes evaluated) and brown adipocyte differentiation (29). We chose to implement >100 nm nanoparticles which is larger than the 20-50 nm range studied to avoid any unexpected side effect. In parallel, liposomes with (DPPC/DMPG/cholesterol/DSPE PEG2K dyed lipid) were made via the standard method of solvent evaporation followed by sonication-assisted hydration in PBS. As a non-water-soluble cargo, FSK loading was carried out in both the MSN core and the lipid shell. Size and zeta potential data of the particle's individual components (MSN, liposomes, liposomes FSK, LCMSN-FSK) with both loading FSK loading capacities (10% and 25%) show the successful formation of a colloidally stable system with a low polydispersity index even with a high FSK content (FIG. 12C) where size of the FSK-LCMSN ranged from 150 nm to 200 nm with more FSK loaded while maintaining a PDI<0.2. The loading of FSK was assessed via TGA (FIG. 12D), where samples were burned up to 700° C. and the weight loss indicated the degradation of each organic component. As the 10% weight loss of the bare MSN is due to water and ethanol release upon continued condensation with the increasing temperature, the FSK-free lipid coating adds ~25% weight to nanoparticles. FSK loading was equivalent to ~15% and ~30% for both samples prepared with 10% and 25% FSK in the liposomes.

2. LCMSNs are Taken Up by Human Mature Adipocytes In Vitro and in Mouse Pre-Isolated Inguinal WAT Ex Vivo without Toxicity

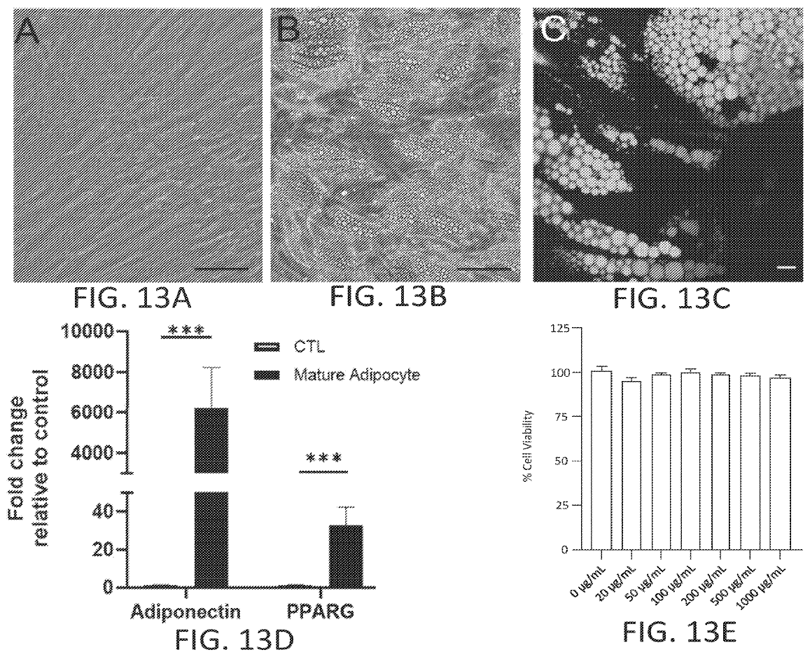

Human adipose derived stem cells (hASC) were differentiated into mature adipocytes. To distinguish mature adipocytes from preadipocytes, their morphology was examined (FIG. 13A-B) alongside the intracytoplasmic buildup of lipid droplets, which was detected by staining with the lipophilic stain BODIPY (FIG. 13C). Gene levels of mature adipocytes such as adiponectin and PPARγ were also evaluated (FIG. 3D). PPARγ is a ligand-dependent transcription factor highly expressed in adipocytes, is a master regulator of adipogenesis and lipid storage (30). Adiponectin is an adipokine secreted by adipocytes and is a well-known homeostatic factor for regulating glucose levels, lipid metabolism, and insulin sensitivity (31). Gene expression of both PPARγ (~6000-fold difference with respect to undifferentiated cells) and adiponectin (~40 times higher than undifferentiated cells), was significantly expressed in mature adipocytes after 14 days of differentiation supporting the successful obtention of human mature adipocytes from undifferentiated hASC. To assess the nanocarrier compatibility, the fully mature adipocytes were treated with the selected LCMSN strategy at concentrations ranging from 20 μg/mL to 1000 μg/mL for 24 hours. The CytoTox-Glo cytotoxicity assay was performed, and the data shows no detectable toxicity even up very high concentrations reaching 1000 μg/mL confirming the compatibility of our LCMSN for drug delivery in adipocytes (FIG. 3D).

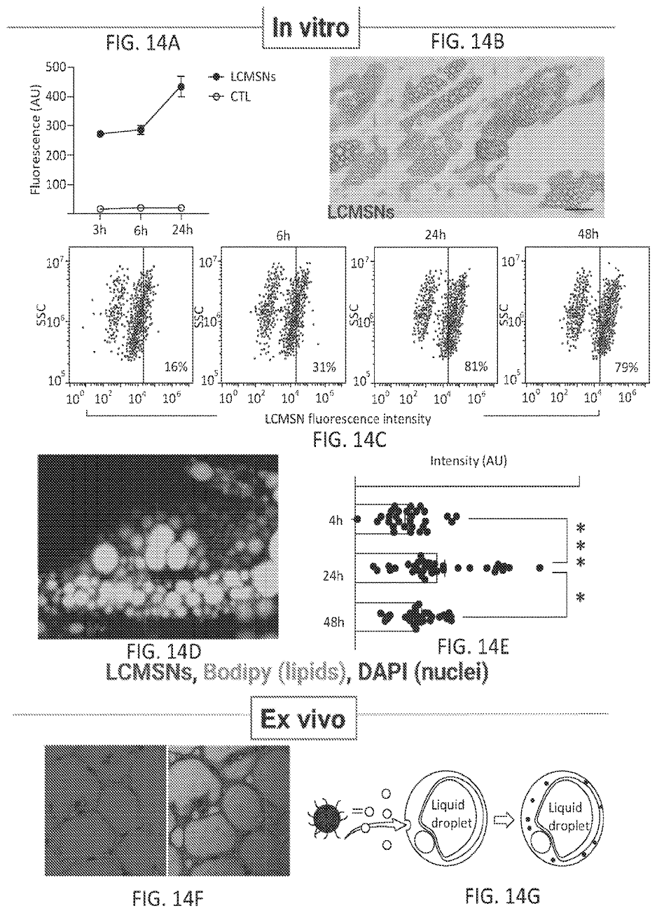

To confirm that the lack of toxicity was not due to lack of internalization, the cellular uptake of LCMSNs in mature adipocytes in vitro was investigated. LCMSNs red fluorescence labeling of both the MSN core and the lipid shell allowed quantitative assessment of cell uptake. LCMSNs uptake was assessed in adipocytes using a robotic microplate reader (6 replicates per group with 3 biological samples). Increased fluorescence was detected in mature adipocytes that were exposed to rhodamine labeled-LCMSNs as early as 3 h after treatment (FIG. 14A). Fluorescence measurement increased with time reaching a maximum at 48 h. Brightfield images indicated rhodamine fluorescence in mature adipocytes and not outside suggesting that LCMSNs are in the cell or attached to the membrane (FIG. 14B). To quantify the percentage of adipocytes with LCMSN uptake, flow cytometry was used. The analysis was performed at different time points (3 h, 6 h, 24 h and 48 h) (FIG. 3C). The number of adipocytes associated with LCMSNs increased with time from ~16% at 3 h to 31% at 6 h before reaching a peak of 80% at 24 that is remaining constant at 48 h (81% at 24 h and 79% at 48 h). Finally, 3D confocal microscopy images were used to confirm LCMSNs internalization in mature adipocytes and to quantify the LCMSNs fluorescent intensity at different time points as a measurement of nanoparticles in the cell. Red LCMSNs were observed around the small lipid droplets of differentiated adipocytes in vitro (FIG. 14D). Quantification of LCMSNs fluorescence intensity per adipocyte showed a maximum uptake after 24 h of treatment with a decline in fluorescence at 48 h (FIG. 14E). suggesting a possible exocytosis occurring after 24 h or the beginning of this process. Uptake of LCMSN in mice pre-isolated inguinal WAT ex vivo.

To understand the behavior of these LCMSNs in adipose tissue, mouse pre-isolated iWAT was incubated with LCMSNs ex vivo for 24 h, followed by confocal microscopy imaging and analysis. In 3D images, LCMSNs were observed surrounding the adipocytes and in the adipocyte nuclei (FIG. 14D), suggesting uptake by mature adipocytes in tissue (FIG. 14). In an effort to quantify the nanoparticle presence in adipocytes, 154 adipocytes (from 10 different images from 2 different animals) were analyzed. A region of interest (ROI) was drawn in proximity to the lipid droplet stained with BODIPY, the fluorescence intensity per adipocyte was binarized, and the percentage of LCMSNs occupation in the adipocyte area was evaluated. After 24 h of treatment an average of 7.24±4.64% of the adipocyte area was occupied by LCMSNs with a maximum of 21.1% and a minimum of 0.2% of area occupation.

### 3. Forskolin Loaded LCMSNs Induce Acute Thermogenesis in Adipocytes In Vitro

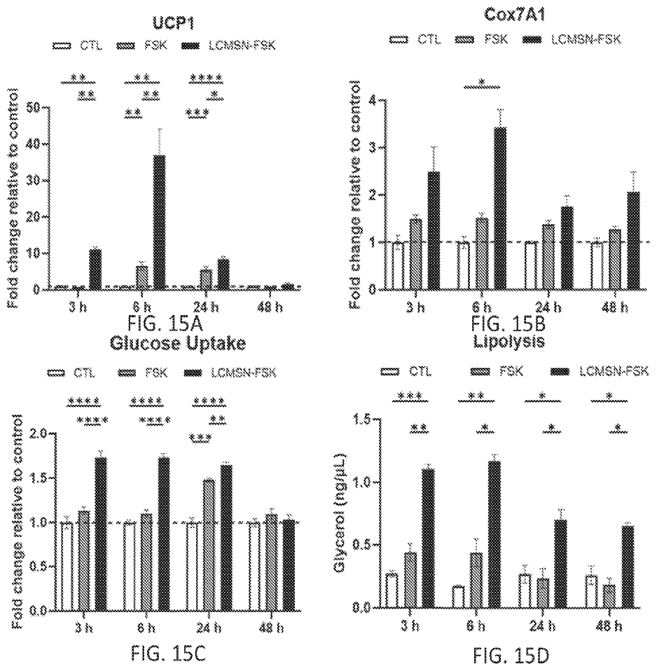

A LCMSN formulation was optimized that does not interfere with adipocyte differentiation and is taken up by mature human adipocytes. We then tested the efficiency of LCMSNs loaded with forskolin to stimulate the expression of thermogenic genes in human adipocytes in vitro. Differentiated human adipocytes were incubated with LCMSNs (±FSK) as well as with free FSK for various times. LCMSNs loaded with FSK significantly increased UCP1 expression, the primary marker of the formation of thermogenic adipocytes. High expression of UCP1 was observed as early as 3 h (p<0.01) after incubation (FIG. 4A). After 6 h of treatment, adipocytes incubated with FSK-LCMSNs showed a 40-fold increase in UCP1 gene expression and other thermogenic genes such as Cox7A1 (3.5×), compared to free forskolin that induced a ~5-fold and 1.5-fold increase respectively (FIG. 15 A-B). At 24 h there was still a significant difference in UCP1 in treated cells compared to CTL, with a greater effect observed in LCMSN-FSK; however, the overall effect was decreased when comparing with UCP1 expression at 3 h and 6h. Upon 48 h incubation, data don't show any significant differences in gene expression amongst all samples. These results suggest an acute response of adipocytes to FSK treatment and therefore the potential of the LCMSNs-FSK in mature human adipocytes thermogenic modulation upon repeated dosing.

4. Metabolic Changes of Adipocytes In Vitro after Treatment with Forskolin Loaded LCMSNs

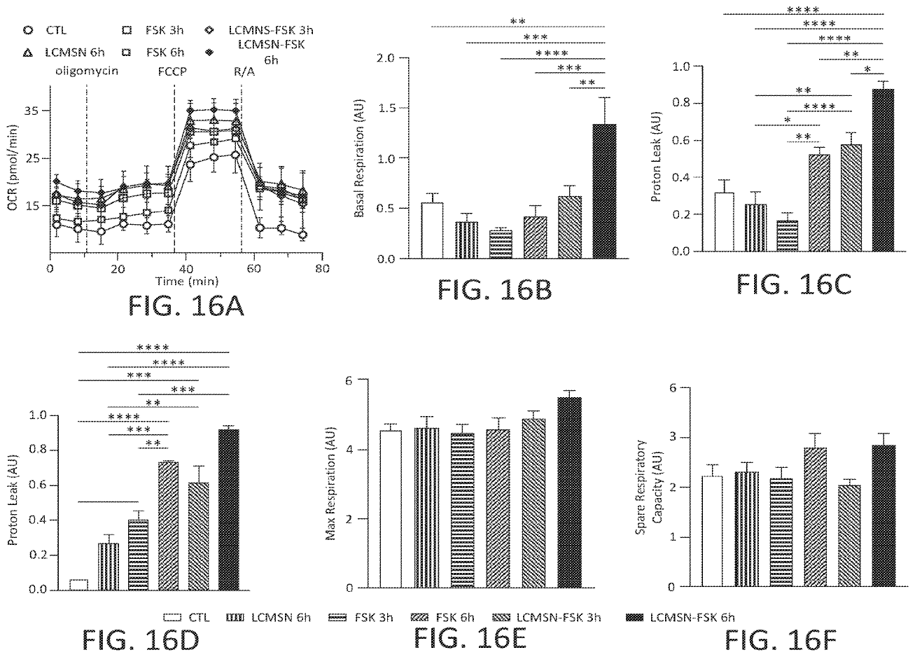

Changes in glucose uptake, lipolysis and oxygen consumption rate (OCR) were evaluated as functional measurements of thermogenic adipocytes after treatment with LCMSNs-FSK. Glucose uptake was significantly increased in adipocytes treated with FSK loaded LCMSNs compared to FSK alone and control (FIG. 15C). Significant differences were observed as early as 3 h (p<0.0001) up to 24 h (p<0.0001) of treatment. Glucose uptake was greater at 3 h and 6 h compared to longer treatments similar to that observed in gene expression. These results indicate that LCMSN-FSK treatment significantly increased the cellular metabolic activities in an acute fashion. Lipolysis was also quantified using a commercially available kit that uses a luminescent method for measuring glycerol. Glycerol is primarily measured as the product of lipolysis where it is released from triglycerides. Activation of thermogenic adipose tissue stimulates lipolysis of fat stored in cytosolic lipid droplets of adipocytes releasing free fatty acids to ignite mitochondrial UCP1 for heat production. We observed a significant increase in lipolysis in adipocytes treated with LCMSNs loaded with FSK at all time points with a greater glycerol expression at 3 h and 6h (FIG. 15D). The oxygen consumption rate OCR was used as a measure of metabolic activity and mitochondrial respiration in the adipocytes after treatment with LCMSN. OCR was measured using standard methods with sequential exposure to oligomycin (inhibits ATP synthase), FCCP (stimulates maximal respiration), and rotenone/antimycin A (inhibits ETC and enables the calculation of nonmitochondrial respiration) over the course of 3 h (FIG. 16A).

Adipocytes treated with LCMSN loaded with FSK for 6h, exhibited the highest basal respiration rate compared to other treatments including FSK alone (FIG. 16B). Proton leak, a measure of uncoupled respiration, and ATP production were also significantly upregulated in adipocytes treated with LCMSN for 6 h (FIGS. 16C and 16D respectively). Differences in ATP capacity indicate variations in the adipocyte's ability to produce and utilize ATP. This could be reflective of changes in energy demand or efficiency. No significant difference was observed in maximum respiration and spare respiratory capacity (FIGS. 16E and 16F respectively). The lack of difference in maximum respiratory capacity could mean that, under maximal stimulation or demand, treated cells exhibit a similar capacity for oxygen consumption. These results suggest that the observed differences are more related to baseline metabolic functions rather than the maximum capacity.

In summary, these studies support that our forskolin-based reported nanosystem (LCMSN-FSK) can enhance expression of thermogenic genes and have a functional effect in metabolic activity suggesting that the effect of forskolin is dramatically enhanced when delivered with the LCMSN technology.

### 5. LCMSNs are Retained in Adipose Tissue Following In Vivo Subcutaneous Injection

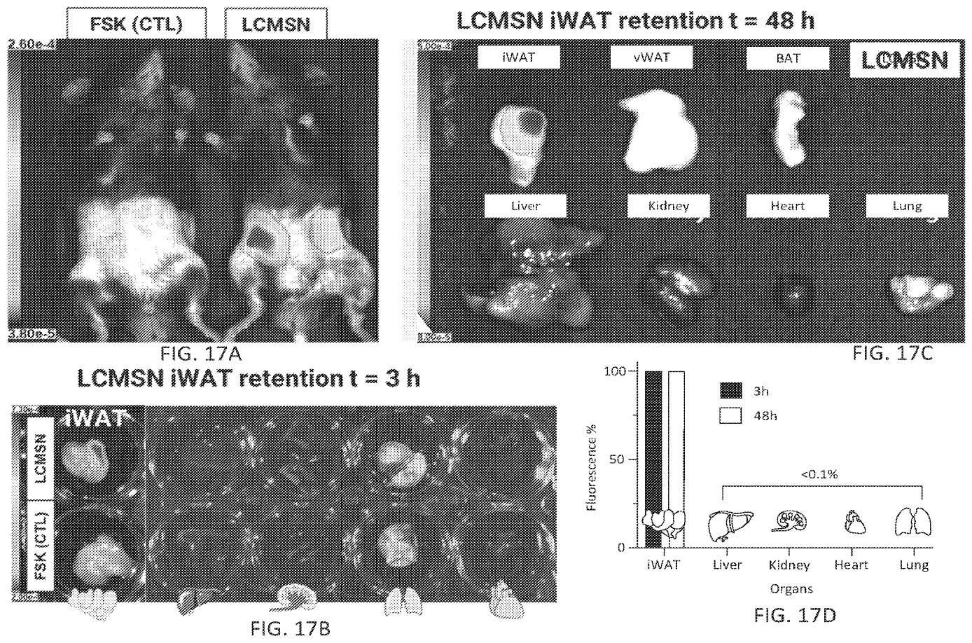

To understand the performance of LCMSNs in vivo, we examined biodistribution using a non-invasive near-infrared (NIR) optical imaging system following subcutaneous injection (FIG. 17A). A near-infrared fluorescence dye (Dylight 633-NHS) was conjugated to the MSN by dissolving 1 mg of DyLight 633 (ThermoFisher) in 1 ml of N,N-dimethyl formamide (DMF: Sigma-Aldrich) and reacted to aminated MSN and then coated by lipids by our standard procedure to form NIR-labeled LCMSNs. In the whole animal imaging, we observed retention of LCMSNs in the iWAT tissue even after 48 h post-injection (FIG. 17). This data supports that LCMSNs are retained in inguinal WAT depots following injection, suggesting they can be used for local adipose delivery while minimizing off target effects. At various time points (3 h, 6 h, 24 h and 48 h) following the injection, mice were euthanized, and a range of tissues namely inguinal and visceral WAT (iWAT, vWAT), liver, lung, kidney, and heart were collected for ex vivo imaging using KINO (FIG. 17B-C). It is important to highlight that the fluorescence signal emitted by the LCMSNs was solely observed in the iWAT, with no signal detected in other non-target tissues (FIG. 17B-D). This outstanding particle retention in the adipose tissue has the advantage of accumulating the FSK into the tissue of interest while minimizing off-target delivery.

6. LCMSNs Loaded with Forskolin Increase Thermogenic Capacity of Adipocytes In Vivo.

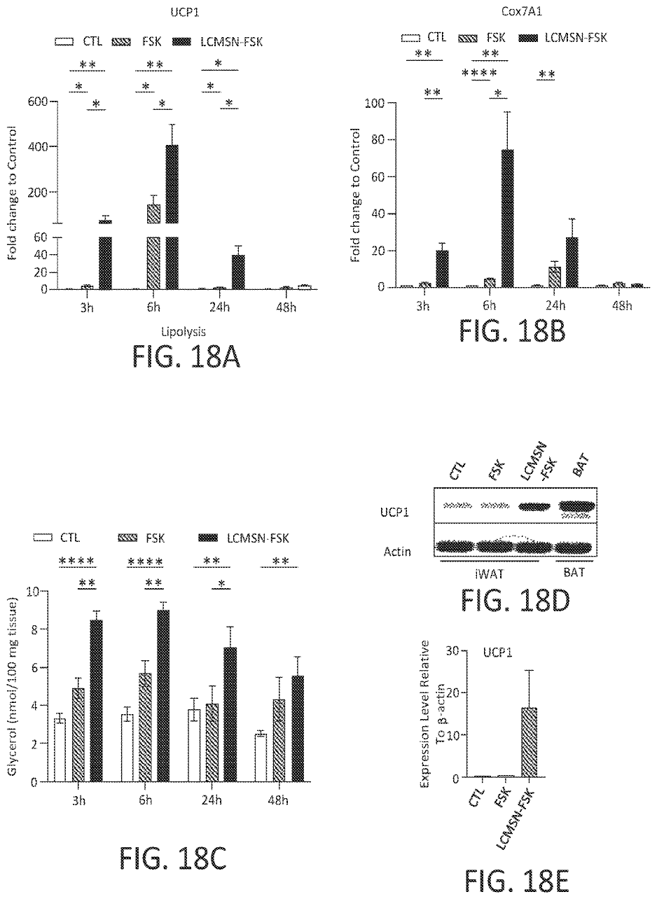

Changes in the thermogenic capacity of adipocytes can be evaluated by the expression of thermogenic genes (UCP1 and Cox7A1). Injection of LCMSNs loaded with forskolin resulted in a significant increase in expression of both thermogenic markers compared to free forskolin and controls as early as 3 h after subcutaneous injection in the iWAT, and differences were observed until 24 h post injection (FIG. 18A, B) in accordance with in vitro studies on human adipocytes that showed the highest increase in thermogenic activity was correlated with shorter treatment duration. Lipolysis was used to evaluate the functional effects of iWAT after treatment with FSK loaded LCMSNs in vivo (FIG. 18C). Data show a significant increase in glycerol content (p<0.0001 for 3 h and 6 h and p<0.001 for 24 h and 48h) supporting that lipolysis in vivo is taking place upon treatment by FSK nanovehicle. UCP1 protein levels were analyzed in isolated iWAT by western blot after 24 h of injection, and results confirmed RT-PCR results, with higher UCP1 levels in iWAT injected with LCMSN loaded with FSK compared to the other treatment groups.

These results demonstrate that one can activate thermogenic activity in adipose tissue in vivo as early as 3 h after injection with LCMSNs loaded with forskolin.
7. LCMSN Loaded with FSK are Much More Effective than Liposomes with FSK and FSK Alone in Preventing Weight Gain

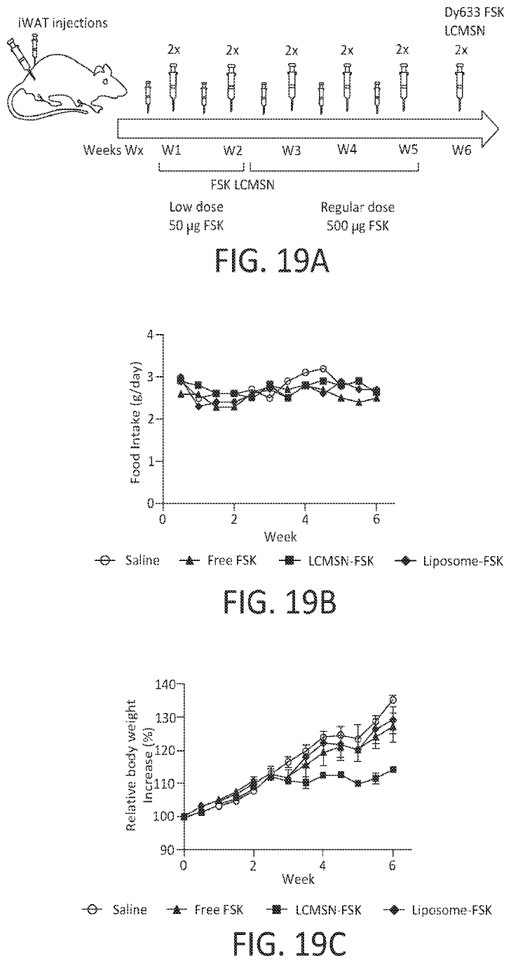

Mice exposed to HFD containing 60% fat, were subcutaneously injected with either saline, free FSK, FSK loaded LCMSN and FSK loaded liposomes as a nanoparticle control. FSK dose was low the first 2 weeks (50 μg/mouse) and then was increased at regular dose (500 μg/mouse) for 4 extra weeks (FIG. 19A). In line with the finding of an acute effect of forskolin on the thermogenic modulation both in vitro and in vivo, we opted for a repeated dose (twice per week). During the study, all mice had a comparable behavior without any noticeable difference in terms of social behavior, anxiety, or food intake (FIG. 19B). At 2 weeks low dose (50 μg per mouse, equivalent to 2.5 mg/kg), there was no noticeable difference in weight amongst all groups, therefore we scaled up our FSK injection dose to 500 μg per mouse (equivalent to 25 mg per kg and to 0.5× dose of Resveratrol per injection). At this dose, the weight gain has been clearly impeded, significant differences in mice body weight were observed after 4.5 weeks of treatment (p<0.05) when comparing the LCMSNs treatment group to the other three treatment groups. This difference in weight gain persisted and increased at the end of the study. At 6 weeks, only mice injected with LCMSNs showed a significantly lower weight gain (15% increase from initial body weight, p<0.001) compared to the other groups that showed a 35% increase (FIG. 19C). These results suggest that by delivering FSK using LCMSNs, the onset of obesity can be significantly and successfully reduced.

### Conclusion.

In summary, provided herein is a novel nanomedicine based on hybrid silica-lipid nanoparticle exhibiting a high capacity for the encapsulation of the naturally extracted forskolin and aiming at modulating both human and murine white adipocytes function by endowing them with an enhanced thermogenesis function. The forskolin-loaded nanoparticle is human adipocyte-biocompatible even at high doses and is efficiently uptaken by mature human adipocytes in vitro and in mice pre-isolated whole fat tissue upon ex vivo incubation. The nanomedicine efficacy is emphasized by a 3-to-40-fold increase of thermogenic capacity reflected by the expression of thermogenic biomarkers with up to 400-fold increase of UCP1 and 80-fold for Cox1A7 as well as metabolic factors namely glycolysis and lipolysis, both in vitro in human cells and in vivo in subcutaneously injected mice. Forskolin has an acute effect on browning with a peak of efficacy at ~6 h after administration. Local in vivo administration of the nanomedicine is characterized by a full retention in the mice inguinal white adipose tissue (iWAT) for at least 48 hours and induces a significant weight gain prevention in high fat diet fed mice (only ~15% weight increase) versus (30-35%) increase when forskolin is used alone or in liposomal carrier with repeated doses within eight weeks of the study. The presented technology will have a transformative impact on the thermogenic capacity of adipose tissue. The capacity of this lipid-silica nanohybrid to hierarchically co-load various agents and biomolecules hides promising potential in targeting complementary browning mechanisms.

### BIBLIOGRAPHY

1. Sun. K., Tordjman, J., Clement, K. & Scherer, P. E. Fibrosis and adipose tissue dysfunction. Cell Metab 18, 470-477 (2013).
2. Sodhi, M., Rezaeianzadeh, R., Kezouh, A. & Etminan, M. Risk of Gastrointestinal Adverse Events Associated With Glucagon-Like Peptide-1 Receptor Agonists for Weight Loss. JAMA 330, 1795-1797 (2023).
3. Efthymiou, V. & Patti, M. E. It Is Not Just Fat: Dissecting the Heterogeneity of Adipose Tissue Function. Curr Diab Rep 22, 177-187 (2022).
4. Lee, M. J., Wu, Y. & Fried, S. K. Adipose tissue heterogeneity: implication of depot differences in adipose tissue for obesity complications. Mol Aspects Med 34, 1-11 (2013).
5. Ikeda, K., Maretich, P. & Kajimura, S. The Common and Distinct Features of Brown and Beige Adipocytes. Trends Endocrinol Metab 29, 191-200 (2018).
6. Wu. J. et al. Beige adipocytes are a distinct type of thermogenic fat cell in mouse and human. Cell 150, 366-376 (2012).
7. Chen, J. Y., Peng, S. Y., Cheng, Y. H., Lee, I. T. & Yu, Y. H. Effect of Forskolin on Body Weight, Glucose Metabolism and Adipocyte Size of Diet-Induced Obesity in Mice. Animals (Basel) 11 (2021).
8. Chechi, K., Carpentier, A. C. & Richard, D. Understanding the brown adipocyte as a contributor to energy homeostasis. Trends Endocrinol Metab 24, 408-420 (2013).
9. Cao, W., Medvedev, A. V., Daniel, K. W. & Collins, S. beta-Adrenergic activation of p38 MAP kinase in adipocytes: cAMP induction of the uncoupling protein 1 (UCP1) gene requires p38 MAP kinase. J Biol Chem 276, 27077-27082 (2001).
10. Lowell, B. B. & Spiegelman, B. M. Towards a molecular understanding of adaptive thermogenesis. Nature 404, 652-660 (2000).
11. Dempersmier. J. et al. Cold-inducible Zfp516 activates UCP1 transcription to promote browning of white fat and development of brown fat. Mol Cell 57, 235-246 (2015).
12. Ohno, H., Shinoda, K., Spiegelman, B. M. & Kajimura, S. PPARgamma agonists induce a white-to-brown fat conversion through stabilization of PRDM16 protein. Cell Metab 15, 395-404 (2012).
13. Whitehead, A. et al. Brown and beige adipose tissue regulate systemic metabolism through a metabolite interorgan signaling axis. Nat Commun 12, 1905 (2021).
14. Richard, A. J., White, U., Elks, C. M. & Stephens, J. M. in Endotext. (eds. K. R. Feingold et al.) (South Dartmouth (MA); 2000).
15. Hiradate, R. et al. A novel dual-targeted rosiglitazone-loaded nanoparticle for the prevention of diet-induced obesity via the browning of white adipose tissue. J Control Release 329, 665-675 (2021).
16. Jiang, C. et al. Dibenzazepine-Loaded Nanoparticles Induce Local Browning of White Adipose Tissue to Counteract Obesity. Mol Ther 25, 1718-1729 (2017).
17. Xue, Y., Xu, X., Zhang, X. Q., Farokhzad, O. C. & Langer, R. Preventing diet-induced obesity in mice by adipose tissue transformation and angiogenesis using targeted nanoparticles. Proc Natl Acad Sci USA 113, 5552-5557 (2016).
18. Zu, Y. et al. Browning white adipose tissue using adipose stromal cell-targeted resveratrol-loaded nanoparticles for combating obesity. J Control Release 333, 339-351 (2021).
19. Yu, B. et al. Targeted delivery of emodin to adipocytes by aptamer-functionalized PEG-PLGA nanoparticles. J Drug Deliv Sci Tec 57 (2020).
20. Scarpace, P. J. & Matheny, M. Thermogenesis in brown adipose tissue with age: post-receptor activation by forskolin. Pflugers Arch 431, 388-394 (1996).
21. Mirbolooki, M. R., Upadhyay, S. K., Constantinescu, C. C., Pan, M. L. & Mukherjee, J. Adrenergic pathway activation enhances brown adipose tissue metabolism: a [(1)(8)F]FDG PET/CT study in mice. Nucl Med Biol 41, 10-16 (2014).
22. Godard, M. P., Johnson, B. A. & Richmond, S. R. Body composition and hormonal adaptations associated with forskolin consumption in overweight and obese men. Obes Res 13, 1335-1343 (2005).
23. Hickerson, R. P. et al. Stability study of unmodified siRNA and relevance to clinical use. Oligonucleotides 18, 345-354 (2008).
24. Hu, B. et al. Therapeutic siRNA: state of the art. Signal Transduct Target Ther 5, 101 (2020).
25. Durfee, P. N. et al. Mesoporous Silica Nanoparticle-Supported Lipid Bilayers (Protocells) for Active Targeting and Delivery to Individual Leukemia Cells. ACS Nano 10, 8325-8345 (2016).
26. Noureddine, A., Marwedel, B., Tang. L., Medina, L. Y. & Serda, R. E. Specific Tumor Localization of Immunogenic Lipid-Coated Mesoporous Silica Nanoparticles following Intraperitoneal Administration in a Mouse Model of Serous Epithelial Ovarian Cancer. Cancers (Basel) 15 (2023).
27. LaBauve, A. E. et al. Lipid-coated mesoporous silica nanoparticles for anti-viral applications via delivery of CRISPR-Cas9 ribonucleoproteins. Sci Rep 13, 6873 (2023).
28. Noureddine, A. et al. Future of Mesoporous Silica Nanoparticles in Nanomedicine: Protocol for Reproducible Synthesis, Characterization, Lipid Coating, and Loading of Therapeutics (Chemotherapeutic, Proteins, siRNA and mRNA). ACS Nano (2023).
29. Son, M. J. et al. Silica nanoparticles inhibit brown adipocyte differentiation via regulation of p38 phosphorylation. Nanotechnology 26, 435101 (2015).
30. Ma, X., Wang, D., Zhao, W. & Xu, L. Deciphering the Roles of PPARgamma in Adipocytes via Dynamic Change of Transcription Complex. Front Endocrinol (Lausanne) 9, 473 (2018).
31. Fu, Y., Luo, N., Klein, R. L. & Garvey, W. T. Adiponectin promotes adipocyte differentiation, insulin sensitivity, and lipid accumulation. J Lipid Res 46, 1369-1379 (2005).
Alkhouli et al., Am. J. Physiol. Endocrinol. Metab., 305:E1427 (2013).
Barczyk et al., Cell Tissue Res., 339:269 (2010).
Bi et al., Nat. Med., 20:911 (2014).
Biener et al., Clin. Chem., 64:108 (2018).
Blumenfeld et al., Sci. Rep., 8:7957 (2018).
Bonnans et al., Nat. Rev. Mol. Cell Biol., 15:786 (2014).
Cannon & Nedergaard, Int. J. Obes. (Lond.), 34:S7 (2010).
Cannon et al., FEBS Lett., 150:129 (1982).
Cawley & Meyerhoefer, J. Health Econ., 31:219 (2012).
Centers for Disease Control and Prevention, (2017).
Centers for Disease Control and Prevention, (2020).
Cordani et al., Stem Cells, 32:874 (2014).
Di Gregorio et al., Diabetes, 54:2305 (2005).
Echtay & Brand, Redox Rep., 12:26 (2007).
Frith et al., Stem Cells Dev., 21:2442 (2012).
Gorski et al., J. Cachexia Sarcopenia Muscle, 9:384 (2018).
Guan et al., Acta Biochim Biophys Sin., 52:116 (2020).
Hynes, Cell, 69:11 (1992).
Ishihama et al., Mol. Cell Proteomics, 4:1265 (2005).
Joe et al., Nat. Cell Biol., 12, 153 (2010).
Kalluri, Nat. Rev. Cancer, 3:422 (2003).
Kubo et al., Vitro Cell Dev. Biol. Anim., 36:38 (2000).
Kuss et al., Acta Biomater., 71:486 (2018).
Lin & Lin, Anal. Biochem., 103:316 (1980).
Lin et al., Biochem. Pharmacol., 119:8 (2016).
Liu et al., Cell Metab., 2:165 (2005).
Malekpour-Dehkordi et al., Gene, 710:406 (2019).
Mariman & Wang, Cell Mol. Life Sci., 67:1277 (2010).
Mielenz et al., J. Biol. Chem., 276:13417 (2001).
Morandi et al., Sci. Rep., 6:28889 (2016).
Moratal et al., Sci. Rep., 8:17005 (2018).
Mon et al., Int. J. Biol. Sci., 10:825 (2014).
Nishimura et al., Diabetes, 56:1517 (2007).
Pope et al., Trends Cell Biol., 26:745 (2016).
Rosenwald et al., Nat. Cell Biol., 15:659 (2013).
Saito et al., Diabetes, 5:1526 (2009).
Schober et al., Exp. Cell Res., 255:303 (2000).
Schulz et al., Proc. Natl. Acad. Sci. USA, 108:143 (2011).
Shen et al., Int. J. Oral. Sci., 3:117 (2011).
Singh et al. Nat. Commun., 11:2758 (2020).
Spiegelman, Diabetes, 62:1774 (2013).
Sun et al., Cancer Res., 79:1239 (2019).
Sun et al., Cell Metab., 18:470 (2013).
Takagi, Curr. Opin. Cell Biol., 19:557 (2007).
Tharp & Stahl, Front. Endocrinol. (Lausanne), 6:164 (2015).
Tharp et al., Cell Metab., 27:602 (2018).
Thyboll et al., Mol. Cell Biol., 22:1194 (2002).
Tran et al., Am. J. Respir. Cell Mol. Biol., 37:668 (2007).
Uezumi et al., Nat. Cell Biol., 12:143 (2010).
Vaicik et al., Endocrinology, 159:356 (2018).
Vaicik et al., J. Mater. Chem. B, 3:7903 (2015).
Vaicik et al., PLoS ONE. 9:e109854 (2014).
Villa-Diaz et al., Stem Cells, 34:1753 (2016).
Wu et al., Genes Dev., 27:234 (2013).
Yu et al., Methods Mol. Biol., 702:29 (2011).
Zingaretti et al., FASEB J., 2:3113 (2009).
Ziober et al., Mol. Biol. Cell. 8:1723 (1997).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A population of protocells comprising a lipid bi- or multi-layer, mesoporous silica nanoparticles (MSNPs), a cargo, and an integrin alpha 7 (ITA7) targeting ligand.

2. A population of protocells comprising a lipid bi- or multi-layer, mesoporous silica nanoparticles (MSNPs) and a cargo, wherein the cargo comprises at least one of forskolin, 3-AR agonist CL-316243, rosiglitazone (Rosi) or GW0742 or peptides, proteins, antibodies, nucleic acids, or drugs that inhibit ITA7.

3. (canceled)

4. The population of claim 1, wherein the targeting ligand is an antibody.

5. The population of claim 1, wherein the targeting ligand is an antibody fragment or a scFv.

6.-10. (canceled)

11. The population of claim 1, wherein the targeting ligand is non-covalently attached to the lipid layer.

12. The population of claim 1, wherein the lipid layer comprises biotin, biotin methyl ester, desthiobiotin, 2-iminobiotin, diamino biotin, biotin carbamate, or biotin carbonate.

13. The population of claim 1, wherein the targeting ligand comprises avidin, streptavidin, neutravidin or bravavidin II.

14. The population of claim 1, wherein the lipid bi- or multi-layer comprises DPPE, DSPC, cholesterol, PEG-DSPC, DODMA, DOTAP, DMPG, or a combination thereof.

15.-16. (canceled)

17. The population of claim 1, wherein the ITA7 targeting ligand comprises monoclonal antibody 334908 or an antigen binding fragment thereof, antibody ab203254 or an antigen binding fragment thereof, E2 or an antigen binding fragment thereof, 8G2 or an antigen binding fragment thereof, PA2226 or an antigen binding fragment thereof, Natalizumab or an antigen binding fragment thereof, Vedolizumab or an antigen binding fragment thereof, AMG-181 or an antigen binding fragment thereof, AJM300 or an antigen binding fragment thereof, Peptide X or an antigen binding fragment thereof or CDP323 or an antigen binding fragment thereof.

18. A pharmaceutical composition comprising the population of claim 1 in combination with a pharmaceutically acceptable carrier, additive and/or excipient.

19. (canceled)

20. A method of treating a metabolic disease comprising administering to a subject in need thereof the pharmaceutical composition of claim 18.

21. A method to transform mammalian white adipose cells to mammalian brown-in-white cells comprising contacting said white adipose cells with an effective amount of the composition of claim 18 to produce said brown-in-white cells.

22. A method to promote transformation of local fat-storage white adipocytes associated with obesity to thermogenic adipocytes comprising contacting said white adipocytes with an effective amount of the composition of claim 18 to produce said thermogenic adipocytes.

23. A method to enhance glucose uptake, lipolysis and/or cellular metabolic activities in mature human adipocytes comprising contacting adipocytes with an effective amount of the composition of claim 18 to enhance glucose uptake and/or lipolysis.

24. A method to prevent weight gain in a subject comprising administering an effective amount of the composition of claim 18 to said subject to prevent weight gain.

25. A method to prevent onset of obesity in a subject comprising administering an effective amount of the composition of claim 18 to said subject, so at prevent the onset of obesity.

26.-28. (canceled)

29. The method of claim 22, wherein the targeting ligand is an antibody, antibody fragment or a scFv.

30. (canceled)

31. The method of claim 22, wherein the cargo comprises peptides, proteins, antibodies, nucleic acids, or a drug.

32. The method of claim 22, wherein the subject is a mammal.

33. (canceled)

34. The method of claim 32, wherein the mammal is obese, has heart disease, has type 2 diabetes, has had a stroke or is at risk for a stroke or a combination thereof.

35.-37. (canceled)
